(19) United States
(12) Patent Application Publication
(10) Pub. No.: US 2024/0115558 A1
(43) Pub. Date: Apr. 11, 2024

(54) ARIMOCLOMOL FOR THE TREATMENT OF NIEMANN PICK DISEASE, TYPE C, IN PATIENTS WITH ER TYPE MISSENSE MUTATIONS

(71) Applicant: ZEVRA DENMARK A/S, Copenhagen N (DK)

(72) Inventors: Thomas Kirkegaard Jensen, Rødovre (DK); Christine l Dali, Copenhagen K (DK); Nikolaj Havnsøe Torp Petersen, Copenhagen Ø (DK); Thomas Blaettler, Gelterkinden (CH); Lærke Clement Freiberg, Kvistgård (DK)

(21) Appl. No.: 18/267,591

(22) PCT Filed: Dec. 23, 2021
(86) PCT No.: PCT/EP2021/087483

§ 371 Date: Jun. 15, 2023

Related U.S. Application Data
(60) Provisional application No. 63/185,111, filed on May 6, 2021.
(60) Provisional application No. 63/130,438, filed on Dec. 24, 2020.

(30) Foreign Application Priority Data
Feb. 5, 2021 (EP) 21155579.2

(51) Int. Cl. A61K 31/444; A61K 31/45; A61P 3/00

(52) U.S. Cl. CPC A61K 31/444; A61K 31/45; A61P 3/00

(57) ABSTRACT

The present invention relates to an active pharmaceutical ingredient selected from N-[2-hydroxy-3-(1-piperidinyl)-propoxy]-pyridine-1-oxide-3-carboximidoyl chloride, its stereoisomers and the acid addition salts thereof; specifically arimoclomol, for use in improved methods of treating Niemann Pick disease, type C (NPC), wherein the patient 5 has an endoplasmic reticulum (ER) type missense mutation in an NPC gene.

### TECHNICAL FIELD

The present invention relates to an active pharmaceutical ingredient selected from N-[2-hydroxy-3-(1-piperidinyl)-propoxy]-pyridine-1-oxide-3-carboximidoyl chloride, its stereoisomers and the acid addition salts thereof; specifically arimoclomol, for use in methods of treating Niemann Pick disease, type C (NPC), wherein the patient has an endoplasmic reticulum (ER) type missense mutation in an NPC gene.

### BACKGROUND

Lysosomal storage diseases (LSD) are a rare group of diseases, characterized by the accumulation of substances in the lysosomal compartment and resulting destabilization hereof, with a resulting devastating effect for affected individuals.

Niemann-Pick disease, type C (NPC) is a rare, progressive, neurodegenerative disease in which lysosomal function is impaired and multiple lipid species accumulate in the lysosomal and endosomal compartments. This lipid accumulation leads to neurodegeneration and visceral organ dysfunction. The clinical presentation and progression of NPC is heterogeneous, depends on age at the time of neurological symptom onset, and includes loss of motor function, swallowing, and speech, as well as cognitive impairment. Individuals with infantile onset of neurological symptoms generally have a more aggressive disease course than patients with juvenile or late-onset disease.

An atypical feature of NPC, relative to other LSDs, is that the accumulated storage material is highly complex and includes multiple different classes of lipids. In NPC, lysosomes accumulate cholesterol and multiple sphingolipids (including glycosphingolipids (GSLs)) and sphingosine, and a reduction in lysosomal calcium.

There is a lack of specific treatment for NPC, and mainly supportive therapies are available. These include medications to control seizures, abnormal posturing of limbs and tremors. Physical, speech and occupational therapy are also used to help with daily functioning. Attempts have been made to correct the cellular changes seen in NPC by using liver or bone marrow transplantation and by cholesterol-lowering medications however, thus far, these have had no effect on delaying the neurologic deterioration or progression of the disease.

The exact genetic and molecular mechanisms underlying NPC have been difficult to discern. Autosomal recessive mutations in either the NPC1 (˜95% of cases) or NPC2 (˜5% of cases) gene, which encode lysosomal proteins essential for the intracellular transport and metabolism of lipids, have been identified to be main genetic causes of NPC. However, there have been numerous different classes of mutations identified in NPC1 and NPC2 in patients with NPC, including missense mutations (70-80%) as well as splicing, frameshift, or premature stop mutations, which are collectively referred to herein as functional null mutations.

Several missense mutations are known to cause NPC. The I1061T mutation is the most commonly reported mutation, and in a homozygous state, or in combination with a functional null allele, it leads to a clinical phenotype with late infantile or early juvenile onset and a ‘classical’ increase of filipin staining in cultured fibroblasts. The mutation results in an NPC1 protein that is misfolded, retained at the endoplasmic reticulum (ER) and subsequently targeted for degradation. Several other mutations found in NPC genes have been reported to result in a similar phenotype, referred to herein as ER type missense mutations.

Despite the identification of these mutations, consistent genotype/phenotype relationships in NPC have been difficult to establish. Moreover, different genotypes/phenotypes have differing responses to treatment, including treatment with miglustat, which has shown only a modest effect on slowing disease progression.

Thus, there exists an urgent need in the art for genotype/patient specific treatments for NPC.

### SUMMARY

The present invention addresses these needs by providing a targeted treatment for subsets of NPC patients, specifically NPC patients with ER type missense mutations.

The inventors have surprisingly found that NPC subjects with at least one ER type missense mutation in at least one of the two alleles in an NPC gene respond robustly to treatment with arimoclomol.

It is an aspect of the present disclosure to provide an active pharmaceutical ingredient selected from N-[2-hydroxy-3-(1-piperidinyl)-propoxy]-pyridine-1-oxide-3-carboximidoyl chloride, its stereoisomers and the acid addition salts thereof; in a particular embodiment arimoclomol ((+)-(R)-N-[2-hydroxy-3-(1-piperidinyl)-propoxy]-pyridine-1-oxide-3-carboximidoyl chloride citrate) for use in a method of treating or preventing Niemann Pick disease, type C (NPC) in a subject, wherein the subject has an endoplasmic reticulum (ER) type missense mutation in an NPC gene.

In some aspects of the present disclosure there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol) in combination with a further active pharmaceutical ingredient, which is N-butyl-deoxynojirimycin (miglustat), for use in a method of treating or preventing Niemann Pick disease, type C (NPC) in a subject, wherein the subject has an ER type missense mutation in an NPC gene.

In particular embodiments the ER type missense mutation of an NPC gene results in an NPC protein that is misfolded, retained at the endoplasmic reticulum (ER) and subsequently targeted for degradation.

In particular embodiments the NPC gene is NPC1, and the NPC protein is NPC1.

It is also an aspect of the present disclosure to provide a method of predicting the responsiveness of a subject with Niemann Pick disease, type C (NPC) to treatment with arimoclomol, optionally in combination with miglustat, the method comprising:
a) determining if the subject has an ER type missense mutation in an NPC gene; and
b) predicting that the subject will respond to treatment with arimoclomol, optionally in combination with miglustat, when the subject is determined to have an ER type missense mutation in an NPC gene.

In some aspects is provided a method of identifying a subject with Niemann Pick disease, type C (NPC) who is likely to be responsive to treatment with arimoclomol, optionally in combination with miglustat, the method comprising:
a) determining if the subject has an ER type missense mutation in an NPC gene; and
b) identifying the subject as being likely to respond to treatment with arimoclomol, optionally in combination with miglustat, when the subject is determined to have an ER type missense mutation in an NPC gene.

### DESCRIPTION OF DRAWINGS

FIG. 1 shows the observed changes of 5-domain NPCCSS scores as compared to baseline at month 12 following treatment with arimoclomol or placebo. (A) Observed changes in 5-domain NPCCSS scores as compared to baseline in the entire population of subjects in the study. (B) Observed changes in 5-domain NPCCSS scores as compared to baseline in subjects who were aged ≥4 years. (C) Observed changes in 5-domain NPCCSS scores as compared to baseline in subjects who were also receiving miglustat. (D) Observed changes in 5-domain NPCCSS scores as compared to baseline in subjects with either a missense/missense or missense/functional null NPC genotype (excluding patients double functional null mutations). In A-D, the solid line represents least-squares mean estimates±standard error based on data obtained while subjects were exposed to study treatment. The mixed model for repeated measures included the main effect of baseline and stratum, respectively, and interaction between treatment and visit. Change from baseline and absolute estimates correspond to the at-baseline overall average subject. Numbers of subject are presented for each time point. (E) Patient-level change in 5-domain NPCCSS scores from baseline to last available data in all subjects in the study. The data are further described in Example 1.

FIG. 2 shows graphs of biomarker analyses in subjects administered arimoclomol or a placebo. (A) Change in HSP70 in PBMCs from months 0 to 12 in arimoclomol-treated patients. (B) Change in unesterified cholesterol level at month 12. (C) Change in serum cholestane-triol level at month 12 (between-group difference: p=0.225). Error bars show the standard error. The data are further described in Example 1.

FIG. 3 shows the relative HSP1A1 expression in NPC fibroblast cells with an ER type genotype treated with various concentrations of arimoclomol. The expression level of HSP1A1 is quantified on days 2 and day 5. The data are further described in Example 2.

FIG. 4 shows quantification of NPC1 protein in arimoclomol treated fibroblast cell lines from NPC patients (50-400 μM arimoclomol for five days). NPC1 protein was quantified by western blotting relative to tubulin (GM18453 & GM18420) or ponceau staining (all other cell lines). Values represent mean+standard deviation (SD) of 3-5 independent experiments as indicated. Statistical analysis was performed by two-way ANOVA with Dunnetts multiple comparison test (p: *<0.05, **<0.01, ***<0.001, ****<0.0001). The data are further described in Example 2.

FIG. 5 shows (A) Western blotting of extracts from NPC fibroblast cell lines harbouring P1007A and a functional null allele (GM18420), or homozygous I1061T NPC1 (GM18453). PNGase cleaves all glycans from the NPC1 protein regardless of maturation status and is included as a control. EndoH sensitive, immature NPC1 is seen in untreated GM18453 extracts. (B) Quantification of the EndoH resistant NPC1, shown as arimoclomol treated relative to untreated cells. Cells were treated for 5 days with 400 μM arimoclomol. Average of three independent experiments, mean±SD. Pairwise t-test (p: *<0.05). The data are further described in Example 2.

### DETAILED DESCRIPTION

### Definitions

The term “pharmaceutically acceptable derivative” in the present context includes pharmaceutically acceptable salts, which indicate a salt which is not harmful to the subjects. Such salts include pharmaceutically acceptable basic or acid addition salts as well as pharmaceutically acceptable metal salts, ammonium salts and alkylated ammonium salts. A pharmaceutically acceptable derivative further includes esters and prodrugs, or other precursors of a compound which may be biologically metabolized into the active compound, or crystal forms of a compound.

The term “acid addition salt” is intended to include “pharmaceutically acceptable acid addition salt” which indicates salts which are not harmful to the subject. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 66, 2, (1977) which is incorporated herein by reference.

The term “therapeutically effective amount” of a compound as used herein refers to an amount sufficient to cure, alleviate, prevent, reduce the risk of, or partially arrest the clinical manifestations of a given disease or disorder and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

For any compound, the therapeutically effective amount can be estimated in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. The dosage may vary within this range depending upon the dosage form employed and sensitivity of the subject.

The term “subject” includes any living organism that has NPC, or is at a risk of developing NPC. In some embodiments, the term “subject” refers to a mammal that has NPC, or is at a risk of developing NPC. In some embodiments, the term subject refers to a human being that has NPC, or is at a risk of developing NPC. The term “patient” is meant to be synonymous and may be used interchangeably with “subject,” unless explicitly indicated otherwise.

Niemann-Pick disease, type C (NPC) is a rare progressive genetic disorder characterized by an inability of the body to transport cholesterol and other fatty substances (lipids) inside of cells. This leads to the abnormal accumulation of these substances within various tissues of the body, including brain tissue.

The terms “treatment” and “treating” as used herein refer to the management and care of an subject for the purpose of combating a condition, disease or disorder. The term is intended to include the full spectrum of treatments for a given condition from which the subject is suffering. The subject to be treated is preferably a mammal, in particular a human being. Treatment of animals, such as mice, rats, dogs, cats, horses, cows, sheep and pigs, is, however, also within the scope of the present context. The subjects to be treated can be of various ages.

It is to be appreciated that references to “treating” or “treatment” include the alleviation of established symptoms of a condition. “Treating” or “treatment” of a state, disorder or condition therefore includes: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

The term “early treatment initiation” refers to starting the administration of arimoclomol as early as possible in the course of NPC in a subject, for example, as soon as the subject has been diagnosed with NPC or during a time period in which the subject exhibits a 5-domain NPCCSS, 17-domain NPCCSS, and/or ASIS score that is below a certain predetermined cutoff value.

As used herein, the term “preventing,” “prevent,” or “protecting against” describes reducing or eliminating the onset of the symptoms or complications of such disease, condition or disorder.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, the arimoclomol can be co-administered with other therapeutic agents. As used herein, the terms “co-administration”, “administered in combination with”, “administered in temporal proximity”, and their grammatical equivalents, are meant to encompass administration of two or more therapeutic agents to a single subject, and are intended to include treatment regimens in which the agents are administered by the same or different route of administration or at the same or different times. In some embodiments arimoclomol will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g., the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating NPC using arimoclomol for guidance.

The compounds or the corresponding pharmaceutical compositions taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the application is formulated to be compatible with its intended route of administration. In some embodiments, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

The term “functional null mutation” refers to a mutation in a gene that results in a truncated and defective protein being produced from the allele that has the functional null mutation. As used herein a functional null mutation is either a frameshift mutation, an aberrant splicing mutation, or a premature stop codon mutation.

The term “frameshift mutation” refers to either a deletion or insertion of any number of nucleotides an allele of a gene in a genome of a subject, wherein the number of nucleotides is not divisible by three, which causes a change in the reading frame of the transcript produced from the allele with the mutation as compared to the transcript produced from a wildtype allele.

The term “aberrant splicing mutation” refers to mutation that changes the splicing activity of a transcript containing the mutation as compared to a transcript with a wildtype sequence.

The term “premature stop mutation” refers to a mutation in a single nucleotide that leads to the creation of a premature stop codon, resulting in the production of a truncated protein product as compared to the wildtype sequence.

The term “missense mutation” refers to one or more nucleotide substitutions in a gene that results in a single amino-acid change in the protein produced by the gene with the mutation as compared to the protein produced by the wildtype gene.

The term “ER type missense mutation” refers to one or more nucleotide substitutions in a gene that results in a single amino-acid change in the protein produced by the gene with the mutation as compared to the protein produced by the wildtype gene. In a particular embodiment the ER type missense mutation of an NPC gene (e.g. NPC1 or NPC2) results in an NPC protein (e.g. NPC1 or NPC2) that is misfolded, retained at the endoplasmic reticulum (ER) and/or subsequently targeted for degradation. Examples of ER type missense mutations and examples of how to classify a mutation as an ER type missense mutation is further provided in Shammas et al., 2019 and Wang et al., 2020 (ER type missense mutations are termed “ER block” and “Class II” in Shammas et al., 2019 and Wang et al., 2020, respectively).

The term “NPC1” refers to the gene encoding the Niemann-Pick type C protein 1, also referred to in the art as the NPC intracellular cholesterol transporter 1. The term “NPC1” refers to the protein product of the NPC1 gene.

The term “NPC2” refers to the gene encoding the Niemann-Pick type C protein 2, also referred to in the art as the NPC intracellular cholesterol transporter 1. The term “NPC2” refers to the protein product of the NPC2 gene.

The term “genotype” refers to the mutational status of both of the alleles of a particular gene locus in a subject. As is known to a person skilled in the art, genotypes are described herein by a mutational description (e.g. missense, functional null, specific mutation description) of the first allele, followed by a “/”, followed by a mutational description (e.g. missense, functional null, specific mutation description) of the second allele.

The term “NPC1 genotype” refers to the genotype of a subject at the NPC1 gene locus.

The term “NPC2 genotype” refers to the genotype of a subject at the NPC2 gene locus.

The term “compound heterozygote” refers to a subject that has two different mutant alleles at a particular gene locus (e.g. at an NPC gene locus such as the NPC1 locus or the NPC2 locus). For example, a subject who has an NPC genotype of “ER type missense”/“ER type missense” would be considered a compound heterozygote if the subject had a first ER type missense mutation on the first NPC allele and a second ER type missense mutation on the second NPC allele, wherein the first missense mutation and the second missense mutation are different.

Mutations on both genome and protein level in this disclosure are labelled according to the 2016 standards of the Human Genome Variation Society (HGVS) and Human genome Organization (HUGO) nomenclature, as would be appreciated by the skilled artisan. DNA mutations are numbered relative to NPC1 cDNA. A single letter amino-acid code is used throughout, frameshift mutations are labelled ‘fs’ after the first affected amino acid, splice mutations are indicated with ‘sp’ after the last presumed correctly translated amino acid and ‘*’ denotes a stop codon.

Mutation nomenclature used herein with respect to nucleotide positions refers to positions within the NPC1 cDNA sequence described in National Library of Medicine entry NM_000271.5:

(SEQ ID NO: 1)
CTTCCTGACCGGCGCGCGCAGCCTGCTGCCGCGGTCAGCGCCTGCTCCTGCTCCTCCGCTCCTC
CTGCGCGGGGTGCTGAAACAGCCCGGGGAAGTAGAGCCGCCTCCGGGGAGCCCAACCAGCCGAA
CGCCGCCGGCGTCAGCAGCCTTGCGCGGCCACAGCATGACCGCTCGCGGCCTGGCCCTTGGCCT
CCTCCTGCTGCTACTGTGTCCAGCGCAGGTGTTTTCACAGTCCTGTGTTTGGTATGGAGAGTGT
GGAATTGCATATGGGGACAAGAGGTACAATTGCGAATATTCTGGCCCACCAAAACCATTGCCAA
AGGATGGATATGACTTAGTGCAGGAACTCTGTCCAGGATTCTTCTTTGGCAATGTCAGTCTCTG
TTGTGATGTTCGGCAGCTTCAGACACTAAAAGACAACCTGCAGCTGCCTCTACAGTTTCTGTCC
AGATGTCCATCCTGTTTTTATAACCTACTGAACCTGTTTTGTGAGCTGACATGTAGCCCTCGAC
AGAGTCAGTTTTTGAATGTTACAGCTACTGAAGATTATGTTGATCCTGTTACAAACCAGACGAA
AACAAATGTGAAAGAGTTACAATACTACGTCGGACAGAGTTTTGCCAATGCAATGTACAATGCC
TGCCGGGATGTGGAGGCCCCCTCAAGTAATGACAAGGCCCTGGGACTCCTGTGTGGGAAGGACG
CTGACGCCTGTAATGCCACCAACTGGATTGAATACATGTTCAATAAGGACAATGGACAGGCACC
TTTTACCATCACTCCTGTGTTTTCAGATTTTCCAGTCCATGGGATGGAGCCCATGAACAATGCC
ACCAAAGGCTGTGACGAGTCTGTGGATGAGGTCACAGCACCATGTAGCTGCCAAGACTGCTCTA
TTGTCTGTGGCCCCAAGCCCCAGCCCCCACCTCCTCCTGCTCCCTGGACGATCCTTGGCTTGGA
CGCCATGTATGTCATCATGTGGATCACCTACATGGCGTTTTTGCTTGTGTTTTTTGGAGCATTT
TTTGCAGTGTGGTGCTACAGAAAACGGTATTTTGTCTCCGAGTACACTCCCATCGATAGCAATA
TAGCTTTTTCTGTTAATGCAAGTGACAAAGGAGAGGCGTCCTGCTGTGACCCTGTCAGCGCAGC
ATTTGAGGGCTGCTTGAGGCGGCTGTTCACACGCTGGGGGTCTTTCTGCGTCCGAAACCCTGGC
TGTGTCATTTTCTTCTCGCTGGTCTTCATTACTGCGTGTTCGTCAGGCCTGGTGTTTGTCCGGG
TCACAACCAATCCAGTTGACCTCTGGTCAGCCCCCAGCAGCCAGGCTCGCCTGGAAAAAGAGTA
CTTTGACCAGCACTTTGGGCCTTTCTTCCGGACGGAGCAGCTCATCATCCGGGCCCCTCTCACT
GACAAACACATTTACCAGCCATACCCTTCGGGAGCTGATGTACCCTTTGGACCTCCGCTTGACA
TACAGATACTGCACCAGGTTCTTGACTTACAAATAGCCATCGAAAACATTACTGCCTCTTATGA
CAATGAGACTGTGACACTTCAAGACATCTGCTTGGCCCCTCTTTCACCGTATAACACGAACTGC
ACCATTTTGAGTGTGTTAAATTACTTCCAGAACAGCCATTCCGTGCTGGACCACAAGAAAGGGG
ACGACTTCTTTGTGTATGCCGATTACCACACGCACTTTCTGTACTGCGTACGGGCTCCTGCCTC
TCTGAATGATACAAGTTTGCTCCATGACCCTTGTCTGGGTACGTTTGGTGGACCAGTGTTCCCG
TGGCTTGTGTTGGGAGGCTATGATGATCAAAACTACAATAACGCCACTGCCCTTGTGATTACCT
TCCCTGTCAATAATTACTATAATGATACAGAGAAGCTCCAGAGGGCCCAGGCCTGGGAAAAAGA
GTTTATTAATTTTGTGAAAAACTACAAGAATCCCAATCTGACCATTTCCTTCACTGCTGAACGA
AGTATTGAAGATGAACTAAATCGTGAAAGTGACAGTGATGTCTTCACCGTTGTAATTAGCTATG
CCATCATGTTTCTATATATTTCCCTAGCCTTGGGGCACATGAAAAGCTGTCGCAGGCTTCTGGT
GGATTCGAAGGTCTCACTAGGCATCGCGGGCATCTTGATCGTGCTGAGCTCGGTGGCTTGCTCC
TTGGGTGTCTTCAGCTACATTGGGTTGCCCTTGACCCTCATTGTGATTGAAGTCATCCCGTTCC
TGGTGCTGGCTGTTGGAGTGGACAACATCTTCATTCTGGTGCAGGCCTACCAGAGAGATGAACG
TCTTCAAGGGGAAACCCTGGATCAGCAGCTGGGCAGGGTCCTAGGAGAAGTGGCTCCCAGTATG
TTCCTGTCATCCTTTTCTGAGACTGTAGCATTTTTCTTAGGAGCATTGTCCGTGATGCCAGCCG
TGCACACCTTCTCTCTCTTTGCGGGATTGGCAGTCTTCATTGACTTTCTTCTGCAGATTACCTG
TTTCGTGAGTCTCTTGGGGTTAGACATTAAACGTCAAGAGAAAAATCGGCTAGACATCTTTTGC
TGTGTCAGAGGTGCTGAAGATGGAACAAGCGTCCAGGCCTCAGAGAGCTGTTTGTTTCGCTTCT
TCAAAAACTCCTATTCTCCACTTCTGCTAAAGGACTGGATGAGACCAATTGTGATAGCAATATT
TGTGGGTGTTCTGTCATTCAGCATCGCAGTCCTGAACAAAGTAGATATTGGATTGGATCAGTCT
CTTTCGATGCCAGATGACTCCTACATGGTGGATTATTTCAAATCCATCAGTCAGTACCTGCATG
CGGGTCCGCCTGTGTACTTTGTCCTGGAGGAAGGGCACGACTACACTTCTTCCAAGGGGCAGAA
CATGGTGTGCGGCGGCATGGGCTGCAACAATGATTCCCTGGTGCAGCAGATATTTAACGCGGCG
CAGCTGGACAACTATACCCGAATAGGCTTCGCCCCCTCGTCCTGGATCGACGATTATTTCGACT
GGGTGAAGCCACAGTCGTCTTGCTGTCGAGTGGACAATATCACTGACCAGTTCTGCAATGCTTC
AGTGGTTGACCCTGCCTGCGTTCGCTGCAGGCCTCTGACTCCGGAAGGCAAACAGAGGCCTCAG
GGGGGAGACTTCATGAGATTCCTGCCCATGTTCCTTTCGGATAACCCTAACCCCAAGTGTGGCA
AAGGGGGACATGCTGCCTATAGTTCTGCAGTTAACATCCTCCTTGGCCATGGCACCAGGGTCGG
AGCCACGTACTTCATGACCTACCACACCGTGCTGCAGACCTCTGCTGACTTTATTGACGCTCTG
AAGAAAGCCCGACTTATAGCCAGTAATGTCACCGAAACCATGGGCATTAACGGCAGTGCCTACC
GAGTATTTCCTTACAGTGTGTTTTATGTCTTCTACGAACAGTACCTGACCATCATTGACGACAC
TATCTTCAACCTCGGTGTGTCCCTGGGCGCGATATTTCTGGTGACCATGGTCCTCCTGGGCTGT
GAGCTCTGGTCTGCAGTCATCATGTGTGCCACCATCGCCATGGTCTTGGTCAACATGTTTGGAG
TTATGTGGCTCTGGGGCATCAGTCTGAACGCTGTATCCTTGGTCAACCTGGTGATGAGCTGTGG
CATCTCCGTGGAGTTCTGCAGCCACATAACCAGAGCGTTCACGGTGAGCATGAAAGGCAGCCGC
GTGGAGCGCGCGGAAGAGGCACTTGCCCACATGGGCAGCTCCGTGTTCAGTGGAATCACACTTA
CAAAATTTGGAGGGATTGTGGTGTTGGCTTTTGCCAAATCTCAAATTTTCCAGATATTCTACTT
CAGGATGTATTTGGCCATGGTCTTACTGGGAGCCACTCACGGATTAATATTTCTCCCTGTCTTA
CTCAGTTACATAGGGCCATCAGTAAATAAAGCCAAAAGTTGTGCCACTGAAGAGCGATACAAAG
GAACAGAGCGCGAACGGCTTCTAAATTTCTAGCCCTCTCGCAGGGCATCCTGACTGAACTGTGT
CTAAGGGTCGGTCGGTTTACCACTGGACGGGTGCTGCATCGGCAAGGCCAAGTTGAACACCGGA
TGGTGCCAACCATCGGTTGTTTGGCAGCAGCTTTGAACGTAGCGCCTGTGAACTCAGGAATGCA
CAGTTGACTTGGGAAGCAGTATTACTAGATCTGGAGGCAACCACAGGACACTAAACTTCTCCCA
GCCTCTTCAGGAAAGAAACCTCATTCTTTGGCAAGCAGGAGGTGACACTAGATGGCTGTGAATG
TGATCCGCTCACTGACACTCTGTAAAGGCCAATCAATGCACTGTCTGTCTCTCCTTTTAGGAGT
AAGCCATCCCACAAGTTCTATACCATATTTTTAGTGACAGTTGAGGTTGTAGATACACTTTATA
ACATTTTATAGTTTAAAGAGCTTTATTAATGCAATAAATTAACTTTGTACACATTTTTATATAA
AAAAACAGCAAGTGATTTCAGAATGTTGTAGGCCTCATTAGAGCTTGGTCTCCAAAAATCTGTT
TGAAAAAAGCAACATGTTCTTCACAGTGTTCCCCTAGAAAGGAAGAGATTTAATTGCCAGTTAG
ATGTGGCATGAAATGAGGGACAAAGAAAGCATCTCGTAGGTGTGTCTACTGGGTTTTAACTTAT
TTTTCTTTAATAAAATACATTGTTTTCCTAAGTTTTGGGGTTACCCTATCTGCTTTGAGAGACA
AATACAAAAGCTAAATGGAAGAGA

Mutation nomenclature used herein with respect to amino acid positions refers to positions within the NPC1 protein sequence described in National Library of Medicine entry NP_000262.2:

(SEQ ID NO: 2)
MTARGLALGLLLLLLCPAQVESQSCVWYGECGIAYGDKRYNCEYSGPPKPLPKDGYDLVQELCP
GFFFGNVSLCCDVRQLQTLKDNLQLPLQFLSRCPSCFYNLLNLFCELTCSPRQSQFLNVTATED
YVDPVTNQTKTNVKELQYYVGQSFANAMYNACRDVEAPSSNDKALGLLCGKDADACNATNWIEY
MFNKDNGQAPFTITPVESDFPVHGMEPMNNATKGCDESVDEVTAPCSCQDCSIVCGPKPQPPPP
PAPWTILGLDAMYVIMWITYMAFLLVFFGAFFAVWCYRKRYFVSEYTPIDSNIAFSVNASDKGE
ASCCDPVSAAFEGCLRRLFTRWGSFCVRNPGCVIFFSLVFITACSSGLVFVRVTTNPVDLWSAP
SSQARLEKEYFDQHFGPFFRTEQLIIRAPLTDKHIYQPYPSGADVPFGPPLDIQILHQVLDLQI
AIENITASYDNETVTLQDICLAPLSPYNTNCTILSVLNYFQNSHSVLDHKKGDDFFVYADYHTH
FLYCVRAPASLNDTSLLHDPCLGTFGGPVFPWLVLGGYDDQNYNNATALVITEPVNNYYNDTEK
LQRAQAWEKEFINFVKNYKNPNLTISFTAERSIEDELNRESDSDVETVVISYAIMFLYISLALG
HMKSCRRLLVDSKVSLGIAGILIVLSSVACSLGVFSYIGLPLTLIVIEVIPFLVLAVGVDNIFI
LVQAYQRDERLQGETLDQQLGRVLGEVAPSMELSSFSETVAFFLGALSVMPAVHTESLFAGLAV
FIDELLQITCFVSLLGLDIKRQEKNRLDIFCCVRGAEDGTSVQASESCLERFFKNSYSPLLLKD
WMRPIVIAIFVGVLSFSIAVLNKVDIGLDQSLSMPDDSYMVDYFKSISQYLHAGPPVYFVLEEG
HDYTSSKGQNMVCGGMGCNNDSLVQQIFNAAQLDNYTRIGFAPSSWIDDYFDWVKPQSSCCRVD
NITDQFCNASVVDPACVRCRPLTPEGKQRPQGGDEMRFLPMELSDNPNPKCGKGGHAAYSSAVN
ILLGHGTRVGATYFMTYHTVLQTSADFIDALKKARLIASNVTETMGINGSAYRVFPYSVFYVFY
EQYLTIIDDTIFNLGVSLGAIFLVTMVLLGCELWSAVIMCATIAMVLVNMFGVMWLWGISLNAV
SLVNLVMSCGISVEFCSHITRAFTVSMKGSRVERAEEALAHMGSSVFSGITLTKFGGIVVLAFA
KSQIFQIFYFRMYLAMVLLGATHGLIFLPVLLSYIGPSVNKAKSCATEERYKGTERERLLNE

The terms “approximately” and “about” as referred herein are synonymous. In some embodiments, “approximately” and “about” refer to the recited amount, value, or duration ±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5% ±0.4%, ±0.3%, ±0.2%, ±0.1%, ±0.09%, ±0.08%, ±0.07%, ±0.06%, ±0.05%, ±0.04%, ±0.03%, ±0.02%, or ±0.01%. In some embodiments, “approximately” and “about” refer to the listed amount, value, or duration ±2.5%, ±2%, ±1.75%, ±1.5%, ±1.25%, ±1%, ±0.9%, ±0.8%, ±0.7%, ±0.6%, ±0.5%. In some embodiments, “approximately” and “about” refer to the listed amount, value, or duration ±1%. In some embodiments, “approximately” and “about” refer to the listed amount, value, or duration ±0.5%. In some embodiments, “approximately” and “about” refer to the listed amount, value, or duration ±0.1%.

### Methods of Treatment

Arimoclomol is an orally available small molecule that crosses the blood-brain barrier, as evidenced by its presence in cerebrospinal fluid of treated patients with amyotrophic lateral sclerosis. The present disclosure pertains, at least in part, to methods for treating Niemann Pick disease, type C, (NPC) in specific clinical subsets of subjects, by administering arimoclomol alone or a combination of arimoclomol and miglustat.

It will be appreciated that reference to arimoclomol herein may include both the free base ((+)-(R)-N-[2-hydroxy-3-(1-piperidinyl)-propoxy]-pyridine-1-oxide-3-carboximidoyl chloride) form, including the acid addition salts thereof, as well as the citrate salt form ((+)-(R)-N-[2-hydroxy-3-(1-piperidinyl)-propoxy]-pyridine-1-oxide-3-carboximidoyl chloride citrate). It is the citrate salt form that is currently investigated in clinical trials.

It is an aspect of the present disclosure to provide an active pharmaceutical ingredient selected from N-[2-hydroxy-3-(1-piperidinyl)-propoxy]-pyridine-1-oxide-3-carboximidoyl chloride, its stereoisomers and the acid addition salts thereof, for use in a method of treating or preventing Niemann Pick disease, type C (NPC) in a subject, wherein the subject has an endoplasmic reticulum (ER) type missense mutation in an NPC gene.

In some aspects of the present disclosure is provided an active pharmaceutical ingredient, which is (+)-(R)-N-[2-hydroxy-3-(1-piperidinyl)-propoxy]-pyridine-1-oxide-3-carboximidoyl chloride, and the acid addition salts thereof, for use in a method of treating or preventing Niemann Pick disease, type C (NPC) in a subject, wherein the subject has an endoplasmic reticulum (ER) type missense mutation in an NPC gene.

In some aspects of the present disclosure is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol), for use in a method of treating or preventing Niemann Pick disease, type C (NPC) in a subject, wherein the subject has an endoplasmic reticulum (ER) type missense mutation in an NPC gene.

It is also an aspect of the present disclosure to provide a method of treating or preventing Niemann Pick disease, type C (NPC) in a subject in need thereof, the method comprising administering a therapeutically effective amount of an active pharmaceutical ingredient selected from N-[2-hydroxy-3-(1-piperidinyl)-propoxy]-pyridine-1-oxide-3-carboximidoyl chloride, its stereoisomers and the acid addition salts thereof to a subject, wherein the subject has an ER type missense mutation in an NPC gene.

In some aspects of the present disclosure is provided the use of an active pharmaceutical ingredient selected from N-[2-hydroxy-3-(1-piperidinyl)-propoxy]-pyridine-1-oxide-3-carboximidoyl chloride, its stereoisomers and the acid addition salts thereof for the manufacture of a medicament for the treatment of NPC in a subject, wherein the subject has an ER type missense mutation in an NPC gene.

In some embodiments, the method further comprises administering a further active pharmaceutical ingredient selected from an N-alkyl derivative of 1,5-dideoxy-1,5-imino-D-glucitol in which said alkyl contains from 2-8 carbon atoms, its stereoisomers and the acid addition salts thereof.

Thus, it is also an aspect of the present disclosure to provide an active pharmaceutical ingredient, which is selected from N-[2-hydroxy-3-(1-piperidinyl)-propoxy]-pyridine-1-oxide-3-carboximidoyl chloride, its stereoisomers and the acid addition salts thereof, in combination with a further active pharmaceutical ingredient, which is selected from an N-alkyl derivative of 1,5-dideoxy-1,5-imino-D-glucitol in which said alkyl contains from 2-8 carbon atoms, its stereoisomers and the acid addition salts thereof, for use in a method of treating or preventing Niemann Pick disease, type C (NPC) in a subject, wherein the subject has an ER type missense mutation in an NPC gene.

In some embodiments, the method further comprises administering a further active pharmaceutical ingredient, which is N-butyl-deoxynojirimycin (miglustat).

In some aspects of the present disclosure is thus provided an active pharmaceutical ingredient, which is (+)-(R)-N-[2-hydroxy-3-(1-piperidinyl)-propoxy]-pyridine-1-oxide-3-carboximidoyl chloride, and the acid addition salts thereof, in combination with a further active pharmaceutical ingredient, which is N-butyl-deoxynojirimycin (miglustat), for use in a method of treating or preventing Niemann Pick disease, type C (NPC) in a subject, wherein the subject has an ER type missense mutation in an NPC gene.

In some aspects of the present disclosure is thus provided an active pharmaceutical ingredient, which is (+)-(R)-N-[2-hydroxy-3-(1-piperidinyl)-propoxy]-pyridine-1-oxide-3-carboximidoyl chloride citrate (arimoclomol) in combination with a further active pharmaceutical ingredient, which is N-butyl-deoxynojirimycin (miglustat), for use in a method of treating or preventing Niemann Pick disease, type C (NPC) in a subject, wherein the subject has an ER type missense mutation in an NPC gene.

It is an aspect of the present disclosure to provide (+)-(R)-N-[2-hydroxy-3-(1-piperidinyl)-propoxy]-pyridine-1-oxide-3-carboximidoyl chloride citrate (arimoclomol) in combination with N-butyl-deoxynojirimycin (miglustat), for use in a method of treating or preventing Niemann Pick disease, type C (NPC) in a subject, wherein the subject has an ER type missense mutation in an NPC gene.

In some embodiments, said treatment is prophylactic. In some embodiments, said treatment is curative. In some embodiments, said treatment is ameliorating.

Progression of NPC in a subject may be tracked by a clinician using the NPC composite clinical severity scale (hereafter “NPCCSS”; see Yanjanin et al.). A full “17-domain NPCCSS score” incorporates clinical signs and symptoms in nine major (ambulation, cognition, eye movement, fine motor, hearing, memory, seizures, speech, swallowing) and eight minor (auditory brainstem response, behavior, gelastic cataplexy, hyperreflexia, incontinence, narcolepsy, psychiatric, respiratory problems) domains to determine a score which describes the severity of the subject's NPC progression (a higher score, the more progressed/severe the disease is). An abridged “5-domain NPCCSS score” is successfully used by clinicians, and incorporates clinical signs and symptoms from the major domains of ambulation, cognition, fine motor, speech and swallowing (see Cortina-Borja).

Subgroups of subjects with a patient population of NPC may also be analyzed using an annual severity increment score (hereafter “ASIS”) which is calculated by dividing the total NPCCSS score by the age of the subject, thereby providing a measure of the rate of disease progression in an individual subject. Accordingly, the resulting value may serve as an index of the annual rate of disease progression (see Cortina-Borja et al).

In one embodiment the subject with NPC has an ASIS between about 0.5 and about 2.

In some embodiments of the present disclosure there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol), for use in a method of treating or preventing Niemann Pick disease, type C (NPC) in a subject, wherein the subject has an endoplasmic reticulum (ER) type missense mutation in an NPC gene and the subject has an ASIS between about 0.5 and about 2.

In some embodiments of the present disclosure there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol), in combination with a further active pharmaceutical ingredient, which is N-butyl-deoxynojirimycin (miglustat), for use in a method of treating or preventing Niemann Pick disease, type C (NPC) in a subject, wherein the subject has an endoplasmic reticulum (ER) type missense mutation in an NPC gene and the subject has an ASIS between about 0.5 and about 2.

In some embodiments, the present disclosure provides a method of treating or preventing NPC in a subject in need thereof, the method comprising administering a therapeutically effective amount of the active pharmaceutical ingredient as disclosed herein to the subject, wherein the subject has an ASIS between about 0.5 and about 2.

In some embodiments, the present disclosure provides a method of treating or preventing NPC in a subject in need thereof, the method comprising administering a therapeutically effective amount of the active pharmaceutical ingredient as disclosed herein and a therapeutically effective amount of the further active pharmaceutical ingredient as disclosed herein to the subject, wherein the subject has an ASIS between about 0.5 and about 2.

Active Pharmaceutical Ingredient (e.g. Arimoclomol)

The present disclosure provides an active pharmaceutical ingredient selected from N-[2-hydroxy-3-(1-piperidinyl)-propoxy]-pyridine-1-oxide-3-carboximidoyl chloride, its stereoisomers and the acid addition salts thereof, for the present purposes.

In some embodiments, the active pharmaceutical ingredient is the racemate of N-[2-hydroxy-3-(1-piperidinyl)-propoxy]-pyridine-1-oxide-3-carboximidoyl chloride.

In some embodiments, the active pharmaceutical ingredient is an optically active stereoisomer of N-[2-hydroxy-3-(1-piperidinyl)-propoxy]-pyridine-1-oxide-3-carboximidoyl chloride.

In some embodiments, the active pharmaceutical ingredient is an enantiomer of N-[2-hydroxy-3-(1-piperidinyl)-propoxy]-pyridine-1-oxide-3-carboximidoyl chloride.

In some embodiments, the active pharmaceutical ingredient is selected from the group consisting of (+)-(R)-N-[2-hydroxy-3-(1-piperidinyl)-propoxy]-pyridine-1-oxide-3-carboximidoyl chloride and (−)-(S)-N-[2-hydroxy-3-(1-piperidinyl)-propoxy]-pyridine-1-oxide-3-carboximidoyl chloride.

In some embodiments, the active pharmaceutical ingredient is selected from the group consisting of (Z)-(R)-N-[2-hydroxy-3-(1-piperidinyl)-propoxy]-pyridine-1-oxide-3-carboximidoyl chloride, (E)-(R)-N-[2-hydroxy-3-(1-piperidinyl)-propoxy]-pyridine-1-oxide-3-carboximidoyl chloride, (Z)-(S)-N-[2-hydroxy-3-(1-piperidinyl)-propoxy]-pyridine-1-oxide-3-carboximidoyl chloride, and (E)-(S)-N-[2-hydroxy-3-(1-piperidinyl)-propoxy]-pyridine-1-oxide-3-carboximidoyl chloride.

In some embodiments, the active pharmaceutical ingredient is an acid addition salt of N-[2-hydroxy-3-(1-piperidinyl)-propoxy]-pyridine-1-oxide-3-carboximidoyl chloride.

In some embodiments, the active pharmaceutical ingredient is selected from the group consisting of N-[2-hydroxy-3-(1-piperidinyl)-propoxy]-pyridine-1-oxide-3-carboximidoyl chloride citrate, and N-[2-hydroxy-3-(1-piperidinyl)-propoxy]-pyridine-1-oxide-3-carboximidoyl chloride maleate.

In some embodiments, the active pharmaceutical ingredient is selected from the group consisting of (+)-(R)-N-[2-hydroxy-3-(1-piperidinyl)-propoxy]-pyridine-1-oxide-3-carboximidoyl chloride citrate, (−)-(S)-N-[2-hydroxy-3-(1-piperidinyl)-propoxy]-pyridine-1-oxide-3-carboximidoyl chloride citrate, (+)-(R)-N-[2-hydroxy-3-(1-piperidinyl)-propoxy]-pyridine-1-oxide-3-carboximidoyl chloride maleate and (−)-(S)-N-[2-hydroxy-3-(1-piperidinyl)-propoxy]-pyridine-1-oxide-3-carboximidoyl chloride maleate.

In some embodiments, the active pharmaceutical ingredient is selected from the group consisting of (2)-(R)-N-[2-hydroxy-3-(1-piperidinyl)-propoxy]-pyridine-1-oxide-3-carboximidoyl chloride citrate, (E)-(R)-N-[2-hydroxy-3-(1-piperidinyl)-propoxy]-pyridine-1-oxide-3-carboximidoyl chloride citrate, (Z)-(S)-N-[2-hydroxy-3-(1-piperidinyl)-propoxy]-pyridine-1-oxide-3-carboximidoyl chloride citrate, (E)-(S)-N-[2-hydroxy-3-(1-piperidinyl)-propoxy]-pyridine-1-oxide-3-carboximidoyl chloride citrate, (2)-(R)-N-[2-hydroxy-3-(1-piperidinyl)-propoxy]-pyridine-1-oxide-3-carboximidoyl chloride maleate, (E)-(R)-N-[2-hydroxy-3-(1-piperidinyl)-propoxy]-pyridine-1-oxide-3-carboximidoyl chloride maleate, (Z)-(S)-N-[2-hydroxy-3-(1-piperidinyl)-propoxy]-pyridine-1-oxide-3-carboximidoyl chloride maleate and (E)-(S)-N-[2-hydroxy-3-(1-piperidinyl)-propoxy]-pyridine-1-oxide-3-carboximidoyl chloride maleate.

In some embodiments, the active pharmaceutical ingredient is arimoclomol, also known as “BRX-345” or (+)-(R)-N-[2-hydroxy-3-(1-piperidinyl)-propoxy]-pyridine-1-oxide-3-carboximidoyl chloride citrate. Arimoclomol, and its preparation are disclosed e.g. in WO 97/16439, WO 00/050403 and WO 01/79174. In some embodiments, arimoclomol is the citrate salt formulation of the free base, i.e. (+)-(R)-N-[2-hydroxy-3-(1-piperidinyl)-propoxy]-pyridine-1-oxide-3-carboximidoyl chloride, which has the structure as shown in formula I:

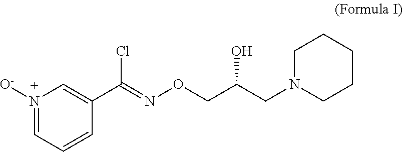

Further Active Pharmaceutical ingredient (e.g. Miglustat)

The present disclosure additionally provides a combination comprising a further active pharmaceutical ingredient selected from an N-alkyl derivative of 1,5-dideoxy-1,5-imino-D-glucitol in which said alkyl contains from 2-8 carbon atoms, its stereoisomers and the acid addition salts thereof, for the present purposes.

In some embodiments, the alkyl group of the further active pharmaceutical ingredient contains from 4-6 carbon atoms.

In some embodiments, the alkyl group of the further active pharmaceutical ingredient is butyl. In some embodiments, the alkyl group of the further active pharmaceutical ingredient is hexyl.

In some embodiments, the further active pharmaceutical ingredient is N-butyl-deoxynojirimycin. N-butyl-deoxynojirimycin is also known as “OGT 918”, N-butylmoranoline, 1,5-(butylimino)-1,5-dideoxy-D-glucitol, (2R,3R,4R,5S)-1-butyl-2-(hydroxymethyl)piperidine-3,4,5-triol or miglustat. In some embodiments, the further active pharmaceutical ingredient thus has the chemical structure of formula II:

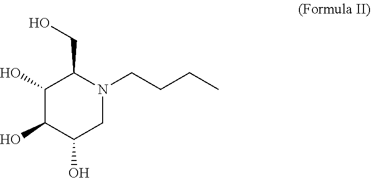

In some embodiments, the further active pharmaceutical ingredient is an acid addition salt of N-butyl-deoxynojirimycin (miglustat).

In some embodiments, the further active pharmaceutical ingredient is Zavesca® or Brazaves®.

In some embodiments, the further active pharmaceutical ingredient is miglustat.

### NPC Patient Subgroups

The inventors have surprisingly found that a specific subgroup of NPC patients, specifically subjects with at least one ER type missense mutation in at least one of the two alleles in an NPC gene, respond robustly to treatment with arimoclomol.

In some embodiments, the ER type missense mutation results in production of an NPC protein that is misfolded, retained at the endoplasmic reticulum (ER) and subsequently targeted for degradation.

In some embodiments, the ER type missense mutation results in production of an NPC protein that is misfolded. In some embodiments, the ER type missense mutation results in production of an NPC protein that is retained at the endoplasmic reticulum (ER). In some embodiments, the ER type missense mutation results in production of an NPC protein that is targeted for degradation. In some embodiments, the ER type missense mutation results in production of an NPC protein that is misfolded, retained at the endoplasmic reticulum (ER) and/or subsequently targeted for degradation.

In some embodiments, the NPC protein is selected from the group consisting of NPC1 and NPC2. In some embodiments, the NPC protein is NPC1. In some embodiments, the NPC protein is NPC2.

In a particular embodiment the NPC protein is NPC1 (SEQ ID NO:2).

In one embodiment there is provided an active pharmaceutical ingredient selected from N-[2-hydroxy-3-(1-piperidinyl)-propoxy]-pyridine-1-oxide-3-carboximidoyl chloride, its stereoisomers and the acid addition salts thereof, for use in a method of treating or preventing Niemann Pick disease, type C (NPC) in a subject, wherein the subject has an NPC protein that is misfolded, retained at the endoplasmic reticulum (ER) and subsequently targeted for degradation. In one embodiment the NPC protein is NPC1.

In one embodiment there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol), for use in a method of treating or preventing Niemann Pick disease, type C (NPC) in a subject, wherein the subject has an NPC protein that is misfolded, retained at the endoplasmic reticulum (ER) and subsequently targeted for degradation. In one embodiment the NPC protein is NPC1.

In one embodiment there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol) in combination with a further active pharmaceutical ingredient, which is N-butyl-deoxynojirimycin (miglustat), for use in a method of treating or preventing Niemann Pick disease, type C (NPC) in a subject, wherein the subject has an NPC protein that is misfolded, retained at the endoplasmic reticulum (ER) and subsequently targeted for degradation. In one embodiment the NPC protein is NPC1.

In some embodiments, the NPC gene is selected from the group consisting of NPC1 and NPC2. In some embodiments, the NPC gene is NPC1. In some embodiments, the NPC gene is NPC2.

In a particular embodiment the NPC gene is NPC1 (SEQ ID NO:1).

In one embodiment there is provided an active pharmaceutical ingredient selected from N-[2-hydroxy-3-(1-piperidinyl)-propoxy]-pyridine-1-oxide-3-carboximidoyl chloride, its stereoisomers and the acid addition salts thereof, for use in a method of treating or preventing Niemann Pick disease, type C (NPC) in a subject, wherein the subject has an endoplasmic reticulum (ER) type missense mutation in the NPC1 gene.

In one embodiment there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol), for use in a method of treating or preventing Niemann Pick disease, type C (NPC) in a subject, wherein the subject has an endoplasmic reticulum (ER) type missense mutation in the NPC1 gene.

In one embodiment there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol) in combination with a further active pharmaceutical ingredient, which is N-butyl-deoxynojirimycin (miglustat), for use in a method of treating or preventing Niemann Pick disease, type C (NPC) in a subject, wherein the subject has an ER type missense mutation in the NPC1 gene.

In some embodiments, the ER type missense mutation results in a single amino-acid change.

In some embodiments, the subject is homozygous for the ER type missense mutation. In some embodiments, the subject is heterozygous for the ER type missense mutation. In some embodiments, the subject has at least one ER type missense mutation in each of the two alleles of the NPC gene. In some embodiments, the subject has at least one ER type missense mutation in one of the two alleles of the NPC gene. In some embodiments, the subject has at least one ER type missense mutation in one of the two alleles of the NPC gene and a functional null mutation in the other allele of the NPC gene. In some embodiments, the subject is a compound heterozygote for the NPC gene.

The I1061T mutation is the most commonly reported mutation that causes NPC, and in a homozygous state, or in combination with a functional null allele, it leads to a clinical phenotype with late infantile or early juvenile onset and a ‘classical’ increase of filipin staining in cultured fibroblasts (Imrie et al. 2007). The mutation results in an NPC1 protein that is misfolded, retained at the endoplasmic reticulum (ER) and subsequently targeted for degradation (Gelsthorpe et al. 2008; Schultz et al. 2018). Given the retained functionality of I1061T NPC1 if transported correctly to the lysosomes (Gelsthorpe et al. 2008), such ER mutations may act in a positive dominant fashion if a treatment was to aid their refolding, maturation and localization to the lysosome.

In one embodiment the ER type missense mutation is selected from the group consisting of C113R, R389L, G535V, L724P, Q921 P, W942C, G1034C, V378A, R404Q, H510P, Q775P, M1142T, N1156S, G1162V, R1186H, L1244P and I1061T.

In one embodiment the ER type missense mutation is selected from the group consisting of I1061T, M1142T, N1156S and R1186H.

In some embodiments, the ER type missense mutation is I1061T. In some embodiments, the ER type missense mutation is M1142T. In some embodiments, the ER type missense mutation is N1156S. In some embodiments, the ER type missense mutation is R1186H.

In some embodiments, the subject has an NPC1 genotype selected from the group consisting of I1061T/E1188*, I1061T/A1151T, I1061T/Q119fs, I1061T/I962fs, T1036M/I1061T, I1061T/V1141G, N968S/R1186H, N1156S/F1199sp2, Q991fs/I1061T, H1016L/I1061T, I1061T/A1192fs, I1061T/N1156S, R1186H/R1186H, P1007A/R1186H, and I1061T/D508fs.

In one embodiment there is provided an active pharmaceutical ingredient selected from N-[2-hydroxy-3-(1-piperidinyl)-propoxy]-pyridine-1-oxide-3-carboximidoyl chloride, its stereoisomers and the acid addition salts thereof, for use in a method of treating or preventing Niemann Pick disease, type C (NPC) in a subject, wherein the subject has an endoplasmic reticulum (ER) type missense mutation in the NPC1 gene selected from the group consisting of C113R, R389L, G535V, L724P, Q921 P, W942C, G1034C, V378A, R404Q, H510P, Q775P, M1142T, N1156S, G1162V, R1186H, L1244P and I1061T;
such as selected from the group consisting of I1061T, M1142T, N1156S and R1186H; such as I1061T;
such as M1142T;
such as N1156S;
such as R1186H.

In one embodiment there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol), for use in a method of treating or preventing Niemann Pick disease, type C (NPC) in a subject, wherein the subject has an endoplasmic reticulum (ER) type missense mutation in the NPC1 gene selected from the group consisting of C113R, R389L, G535V, L724P, Q921 P, W942C, G1034C, V378A, R4040, H510P, Q775P, M1142T, N1156S, G1162V, R1186H, L1244P and I1061T;
such as selected from the group consisting of I1061T, M1142T, N1156S and R1186H;
such as I1061T;
such as M1142T;
such as N1156S;

such as R1186H.

In one embodiment there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol) in combination with a further active pharmaceutical ingredient, which is N-butyl-deoxynojirimycin (miglustat), for use in a method of treating or preventing Niemann Pick disease, type C (NPC) in a subject, wherein the subject has an ER type missense mutation in the NPC1 gene selected from the group consisting of C113R, R389L, G535V, L724P, Q921 P, W942C, G1034C, V378A, R404Q, H510P, Q775P, M1142T, N1156S, G1162V, R1186H, L1244P and I1061T;
such as selected from the group consisting of I1061T, M1142T, N1156S and R1186H;
such as I1061T;
such as M1142T;
such as N1156S;
such as R1186H.

In one embodiment there is provided an active pharmaceutical ingredient selected from N-[2-hydroxy-3-(1-piperidinyl)-propoxy]-pyridine-1-oxide-3-carboximidoyl chloride, its stereoisomers and the acid addition salts thereof, for use in a method of treating or preventing Niemann Pick disease, type C (NPC) in a subject, wherein the subject has an NPC1 genotype selected from the group consisting of I1061T/E1188*, i1061T/A1151T, I1061T/Q119fs, I1061T/I962fs, T1036M/I1061T, I1061T/V1141G, N968S/R1186H, N1156S/F1199sp2, Q991fs/I1061T, H1016L/I1061T, I1061T/A1192fs, I1061T/N1156S, R1186H/R1186H, P1007A/R1186H, and I1061T/D508fs.

In one embodiment there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol), for use in a method of treating or preventing Niemann Pick disease, type C (NPC) in a subject, wherein the subject has an NPC1 genotype selected from the group consisting of I1061T/E1188*, I1061T/A1151T, I1061T/Q119fs, I1061T/I962fs, T1036M/I1061T, I1061T/V1141G, N968S/R1186H, N1156S/F1199sp2, Q991fs/I1061T, H1016L/I1061T, I1061T/A1192fs, I1061T/N1156S, R1186H/R1186H, P1007A/R1186H, and I1061T/D508fs.

In one embodiment there is provided an active pharmaceutical ingredient, which is (+)-(R)-N-[2-hydroxy-3-(1-piperidinyl)-propoxy]-pyridine-1-oxide-3-carboximidoyl chloride citrate (arimoclomol) in combination with a further active pharmaceutical ingredient, which is N-butyl-deoxynojirimycin (miglustat), for use in a method of treating or preventing Niemann Pick disease, type C (NPC) in a subject, wherein the subject has an NPC1 genotype selected from the group consisting of I1061T/E1188*, I1061T/A1151T, I1061T/Q119fs, I1061T/I962fs, T1036M/I1061T, I1061T/V1141G, N968S/R1186H, N1156S/F1199sp2, Q991fs/I1061T, H1016L/I1061T, I1061T/A1192fs, I1061T/N1156S, R1186H/R1186H, P1007A/R1186H, and I1061T/D508fs.

In some embodiments, determining an NPC genotype (e.g. an NPC1 genotype and/or an NPC2 genotype) comprises sequencing the nucleic acid isolated from a biological sample from the subject. As would be appreciated by the person skilled in the art, an NPC genotype (e.g. an NPC1 genotype and/or an NPC2 genotype) can be determined using any genotyping method known in the art.

In some embodiments, the subject or patient is a mammal. In some embodiments, the subject or patient is a human.

In some embodiments, the subject or patient is about 1 year or older, such as about 2 years or older, such as about 3 years or older, such as about 4 years or older, such as about 5 years or older, such as about 6 years or older, such as about 7 years or older, such as about 8 years or older, such as about 9 years or older, such as about 10 years or older. In a particular embodiment, the subject or patient is about 4 years or older.

In some embodiments, the subject is about 1 year old. In some embodiments, the subject is about 2 years old. In some embodiments, the subject is about 3 years old. In some embodiments, the subject is about 4 years old. In some embodiments, the subject is about 5 years old. In some embodiments, the subject is about 6 years old. In some embodiments, the subject is about 7 years old. In some embodiments, the subject is about 8 years old. In some embodiments, the subject is about 9 years old. In some embodiments, the subject is about 10 years old. In some embodiments, the subject is about 11 years old. In some embodiments, the subject is about 12 years old. In some embodiments, the subject is about 13 years old. In some embodiments, the subject is about 14 years old. In some embodiments, the subject is about 15 years old. In some embodiments, the subject is about 16 years old. In some embodiments, the subject is about 17 years old. In some embodiments, the subject is about 18 years old. In some embodiments, the subject is about 19 years old. In some embodiments, the subject is about 20 years old. In some embodiments, the subject is about 21 years old. In some embodiments, the subject is about 22 years old. In some embodiments, the subject is about 23 years old. In some embodiments, the subject is about 24 years old. In some embodiments, the subject is about 25 years old. In some embodiments, the subject is about 26 years old. In some embodiments, the subject is about 27 years old. In some embodiments, the subject is about 28 years old. In some embodiments, the subject is about 29 years old. In some embodiments, the subject is about 30 years old.

In some embodiments, the subject is between the ages of about 0 years old to about 5 years old. In some embodiments, the subject is between the ages of about 5 years old to about 10 years old. In some embodiments, the subject is between the ages of about 10 years old to about 15 years old. In some embodiments, the subject is between the ages of about 15 years old to about 20 years old. In some embodiments, the subject is between the ages of about 20 years old to about 30 years old. In some embodiments, the subject is between the ages of about 30 years old to about 40 years old. In some embodiments, the subject is between the ages of about 40 years old to about 50 years old. In some embodiments, the subject is between the ages of about 50 years old to about 60 years old. In some embodiments, the subject is between the ages of about 60 years old to about 70 years old. In some embodiments, the subject is between the ages of about 70 years old to about 80 years old. In some embodiments, the subject is between the ages of about 80 years old to about 90 years old. In some embodiments, the subject is between the ages of about 90 years old to about 100 years old. In some embodiments, the subject is between the ages of about 100 years old to about 110 years old.

### Formulation

Whilst it is possible for the active pharmaceutical ingredient to be administered as the raw chemical, it is in some embodiments preferred to present them in the form of a pharmaceutical formulation.

Accordingly, also provided herewith is a composition, such as a pharmaceutical composition, i.e. a pharmaceutically safe composition, comprising the active pharmaceutical ingredient as defined herein for use in a method of treating or preventing NPC in a subject, wherein the subject has an ER type missense mutation in an NPC gene.

Also provided herewith is a composition, such as a pharmaceutical composition, i.e. a pharmaceutically safe composition, comprising, separately or together, an active pharmaceutical ingredient as defined herein and a further active pharmaceutical ingredients as defined herein.

The composition in some embodiments further comprises a pharmaceutically and/or physiologically acceptable diluent, carrier and/or excipients.

In some embodiments, the composition is formulated for oral administration, such as in the form of tablets or capsules, or such as an oral powder, such as an oral powder suitable for suspension in a liquid, or such as as a suspension for oral administration.

In some embodiments, the composition is formulated as a liquid for injection.

Pharmaceutical compositions containing the active and, optionally, the further active pharmaceutical ingredients of the present invention may be prepared by conventional techniques.

### Administration and Dosage

The active pharmaceutical ingredient as disclosed herein, or a composition comprising the same as defined herein, is in some embodiments administered to a subject in need thereof in pharmaceutically effective doses or in a therapeutically effective amount.

In some embodiments, the active pharmaceutical ingredient as disclosed herein is administered for more than 1 week, such as for more than 2 weeks, such as for more than 3 weeks, such as for more than 4 weeks.

In some embodiments, the active pharmaceutical ingredient as disclosed herein is administered for more than 1 month, such as for more than 2 months, such as for more than 3 months, such as for more than 4 months, such as for more than 5 months, such as for more than 6 months.

In some embodiments, the active pharmaceutical ingredient as disclosed herein is administered for more than 1 year, such as for more than two years, such as for more than 3 years, such as for more than 4 years, such as for 5 years or more.

In some embodiments, treatment comprises early treatment initiation with the active pharmaceutical ingredient as disclosed herein. In other words, in some embodiments, treatment with the with the active pharmaceutical ingredient as disclosed herein is initiated soon after presentation of symptoms and/or diagnosis of NPC.

In one embodiment, the dosages are calculated on the basis of the arimoclomol citrate salt ((+)-(R)-N-[2-hydroxy-3-(1-piperidinyl)-propoxy]-pyridine-1-oxide-3-carboximidoyl chloride citrate).

In one embodiment, the dosages are calculated on the basis of the arimoclomol free base ((+)-(R)-N-[2-hydroxy-3-(1-piperidinyl)-propoxy]-pyridine-1-oxide-3-carboximidoyl chloride).

In some embodiments, the active pharmaceutical ingredient is administered as described in Table A.

In some embodiments, the active pharmaceutical ingredient is administered as described in Table B.

TABLE A

| | | | |
|---|---|---|---|
| Subject | Dosage | Dosage | Administration |
| Weight | (free base) | (citrate) | Schedule |

| | | | | | |
|---|---|---|---|---|---|
| 8 kg to 15 kg | 31 | mg | 50 | mg | t.i.d |
| 15 kg to 22 kg | 47 | mg | 75 | mg | t.i.d |
| 22 kg to 38 kg | 62 | mg | 100 | mg | t.i.d |
| 38 kg to 55 kg | 93 | mg | 150 | mg | t.i.d |
| >55 kg | 124 | mg | 200 | mg | t.i.d |

TABLE B

| | | | |
|---|---|---|---|
| Subject | Dosage | Dosage | Administration |
| Weight | (free base) | (citrate) | Schedule |

| | | | | | |
|---|---|---|---|---|---|
| 8 kg to 15 kg | 47 | mg | 75 | mg | t.i.d |
| 15 kg to 30 kg | 62 | mg | 100 | mg | t.i.d |
| 30 kg to 55 kg | 93 | mg | 150 | mg | t.i.d |
| >55 kg | 124 | mg | 200 | mg | t.i.d |

### Dosages, Arimoclomol (Citrate Salt)

In some embodiments, the active pharmaceutical ingredient as disclosed herein is administered from about 25 mg/day to about 1000 mg/day. In a preferred embodiment the active pharmaceutical ingredient is arimoclomol ((+)-(R)-N-[2-hydroxy-3-(1-piperidinyl)-propoxy]-pyridine-1-oxide-3-carboximidoyl chloride citrate).

In some embodiments, the active pharmaceutical ingredient as disclosed herein is administered from about 100 mg/day to about 1000 mg/day. In some embodiments, the active pharmaceutical ingredient as disclosed herein is administered from about 150 mg/day to about 1000 mg/day. In some embodiments, the active pharmaceutical ingredient as disclosed herein is administered from about 200 mg/day to about 1000 mg/day. In some embodiments, the active pharmaceutical ingredient as disclosed herein is administered from about 250 mg/day to about 1000 mg/day. In some embodiments, the active pharmaceutical ingredient as disclosed herein is administered from about 300 mg/day to about 1000 mg/day. In some embodiments, the active pharmaceutical ingredient as disclosed herein is administered from about 350 mg/day to about 1000 mg/day. In some embodiments, the active pharmaceutical ingredient as disclosed herein is administered from about 400 mg/day to about 1000 mg/day. In some embodiments, the active pharmaceutical ingredient as disclosed herein is administered from about 450 mg/day to about 1000 mg/day. In some embodiments, the active pharmaceutical ingredient as disclosed herein is administered from about 500 mg/day to about 1000 mg/day. In some embodiments, the active pharmaceutical ingredient as disclosed herein is administered from about 550 mg/day to about 1000 mg/day. In some embodiments, the active pharmaceutical ingredient as disclosed herein is administered from about 600 mg/day to about 1000 mg/day. In some embodiments, the active pharmaceutical ingredient as disclosed herein is administered from about 650 mg/day to about 1000 mg/day. In some embodiments, the active pharmaceutical ingredient as disclosed herein is administered from about 700 mg/day to about 1000 mg/day. In some embodiments, the active pharmaceutical ingredient as disclosed herein is administered from about 750 mg/day to about 1000 mg/day. In some embodiments, the active pharmaceutical ingredient as disclosed herein is administered from about 800 mg/day to about 1000 mg/day. In some embodiments, the active pharmaceutical ingredient as disclosed herein is administered from about 850 mg/day to about 1000 mg/day. In some embodiments, the active pharmaceutical ingredient as disclosed herein is administered from about 900 mg/day to about 1000 mg/day. In some embodiments, the active pharmaceutical ingredient as disclosed herein is administered from about 950 mg/day to about 1000 mg/day. In preferred embodiments, the active pharmaceutical ingredient is arimoclomol.

In some embodiments, the active pharmaceutical ingredient as disclosed herein is administered from about 100 mg/day to about 950 mg/day. In some embodiments, the active pharmaceutical ingredient as disclosed herein is administered from about 100 mg/day to about 900 mg/day. In some embodiments, the active pharmaceutical ingredient as disclosed herein is administered from about 100 mg/day to about 850 mg/day. In some embodiments, the active pharmaceutical ingredient as disclosed herein is administered from about 100 mg/day to about 800 mg/day. In some embodiments, the active pharmaceutical ingredient as disclosed herein is administered from about 100 mg/day to about 750 mg/day. In some embodiments, the active pharmaceutical ingredient as disclosed herein is administered from about 100 mg/day to about 700 mg/day. In some embodiments, the active pharmaceutical ingredient as disclosed herein is administered from about 100 mg/day to about 650 mg/day. In some embodiments, the active pharmaceutical ingredient as disclosed herein is administered from about 100 mg/day to about 600 mg/day. In some embodiments, the active pharmaceutical ingredient as disclosed herein is administered from about 100 mg/day to about 550 mg/day. In some embodiments, the active pharmaceutical ingredient as disclosed herein is administered from about 100 mg/day to about 500 mg/day. In some embodiments, the active pharmaceutical ingredient as disclosed herein is administered from about 100 mg/day to about 450 mg/day. In some embodiments, the active pharmaceutical ingredient as disclosed herein is administered from about 100 mg/day to about 400 mg/day. In some embodiments, the active pharmaceutical ingredient as disclosed herein is administered from about 100 mg/day to about 350 mg/day. In some embodiments, the active pharmaceutical ingredient as disclosed herein is administered from about 100 mg/day to about 300 mg/day. In some embodiments, the active pharmaceutical ingredient as disclosed herein is administered from about 100 mg/day to about 250 mg/day. In some embodiments, the active pharmaceutical ingredient as disclosed herein is administered from about 100 mg/day to about 200 mg/day. In some embodiments, the active pharmaceutical ingredient as disclosed herein is administered from about 100 mg/day to about 150 mg/day. In preferred embodiments, the active pharmaceutical ingredient is (+)-(R)-N-[2-hydroxy-3-(1-piperidinyl)-propoxy]-pyridine-1-oxide-3-carboximidoyl chloride citrate (arimoclomol).

In some embodiments, the active pharmaceutical ingredient as disclosed herein is administered at about 100 mg/day. In some embodiments, the active pharmaceutical ingredient as disclosed herein is administered at about 125 mg/day. In some embodiments, the active pharmaceutical ingredient as disclosed herein is administered at about 150 mg/day. In some embodiments, the active pharmaceutical ingredient as disclosed herein is administered at about 175 mg/day. In some embodiments, the active pharmaceutical ingredient as disclosed herein is administered at about 200 mg/day. In some embodiments, the active pharmaceutical ingredient as disclosed herein is administered at about 225 mg/day. In some embodiments, the active pharmaceutical ingredient as disclosed herein is administered at about 250 mg/day. In some embodiments, the active pharmaceutical ingredient as disclosed herein is administered at about 275 mg/day. In some embodiments, the active pharmaceutical ingredient as disclosed herein is administered at about 300 mg/day. In some embodiments, the active pharmaceutical ingredient as disclosed herein is administered at about 325 mg/day. In some embodiments, the active pharmaceutical ingredient as disclosed herein is administered at about 350 mg/day. In some embodiments, the active pharmaceutical ingredient as disclosed herein is administered at about 375 mg/day. In some embodiments, the active pharmaceutical ingredient as disclosed herein is administered at about 400 mg/day. In some embodiments, the active pharmaceutical ingredient as disclosed herein is administered at about 425 mg/day. In some embodiments, the active pharmaceutical ingredient as disclosed herein is administered at about 450 mg/day. In some embodiments, the active pharmaceutical ingredient as disclosed herein is administered at about 475 mg/day. In some embodiments, the active pharmaceutical ingredient as disclosed herein is administered at about 500 mg/day. In some embodiments, the active pharmaceutical ingredient as disclosed herein is administered at about 525 mg/day. In some embodiments, the active pharmaceutical ingredient as disclosed herein is administered at about 550 mg/day. In some embodiments, the active pharmaceutical ingredient as disclosed herein is administered at about 575 mg/day. In some embodiments, the active pharmaceutical ingredient as disclosed herein is administered at about 600 mg/day. In some embodiments, the active pharmaceutical ingredient as disclosed herein is administered at about 625 mg/day. In some embodiments, the active pharmaceutical ingredient as disclosed herein is administered at about 650 mg/day. In some embodiments, the active pharmaceutical ingredient as disclosed herein is administered at about 675 mg/day. In some embodiments, the active pharmaceutical ingredient as disclosed herein is administered at about 700 mg/day. In preferred embodiments, the active pharmaceutical ingredient is arimoclomol.

In some embodiments, the active pharmaceutical ingredient as disclosed herein is administered in doses of about 25 mg to about 300 mg. In some embodiments, the active pharmaceutical ingredient as disclosed herein is administered in doses of about 50 mg to about 300 mg. In some embodiments, the active pharmaceutical ingredient as disclosed herein is administered in doses of about 75 mg to about 300 mg. In some embodiments, the active pharmaceutical ingredient as disclosed herein is administered in doses of about 100 mg to about 300 mg. In some embodiments, the active pharmaceutical ingredient as disclosed herein is administered in doses of about 125 mg to about 300 mg. In some embodiments, the active pharmaceutical ingredient as disclosed herein is administered in doses of about 150 mg to about 300 mg. In some embodiments, the active pharmaceutical ingredient as disclosed herein is administered in doses of about 175 mg to about 300 mg. In some embodiments, the active pharmaceutical ingredient as disclosed herein is administered in doses of about 200 mg to about 300 mg. In some embodiments, the active pharmaceutical ingredient as disclosed herein is administered in doses of about 225 mg to about 300 mg. In some embodiments, the active pharmaceutical ingredient as disclosed herein is administered in doses of about 250 mg to about 300 mg. In some embodiments, the active pharmaceutical ingredient as disclosed herein is administered in doses of about 275 mg to about 300 mg. In preferred embodiments, the active pharmaceutical ingredient is arimoclomol.

In some embodiments, the active pharmaceutical ingredient as disclosed herein is administered in doses of about 25 mg to about 275 mg. In some embodiments, the active pharmaceutical ingredient as disclosed herein is administered in doses of about 25 mg to about 250 mg. In some embodiments, the active pharmaceutical ingredient as disclosed herein is administered in doses of about 25 mg to about 225 mg. In some embodiments, the active pharmaceutical ingredient as disclosed herein is administered in doses of about 25 mg to about 200 mg. In some embodiments, the active pharmaceutical ingredient as disclosed herein is administered in doses of about 25 mg to about 175 mg. In some embodiments, the active pharmaceutical ingredient as disclosed herein is administered in doses of about 25 mg to about 150 mg. In some embodiments, the active pharmaceutical ingredient as disclosed herein is administered in doses of about 25 mg to about 125 mg. In some embodiments, the active pharmaceutical ingredient as disclosed herein is administered in doses of about 25 mg to about 100 mg. In some embodiments, the active pharmaceutical ingredient as disclosed herein is administered in doses of about 25 mg to about 75 mg. In some embodiments, the active pharmaceutical ingredient as disclosed herein is administered in doses of about 25 mg to about 50 mg. In preferred embodiments, the active pharmaceutical ingredient is arimoclomol.

In some embodiments, the active pharmaceutical ingredient as disclosed herein is administered in doses of about 25 mg. In some embodiments, the active pharmaceutical ingredient as disclosed herein is administered in doses of about 50 mg. In some embodiments, the active pharmaceutical ingredient as disclosed herein is administered in doses of about 75 mg. In some embodiments, the active pharmaceutical ingredient as disclosed herein is administered in doses of about 100 mg. In some embodiments, the active pharmaceutical ingredient as disclosed herein is administered in doses of about 125 mg. In some embodiments, the active pharmaceutical ingredient as disclosed herein is administered in doses of about 150 mg. In some embodiments, the active pharmaceutical ingredient as disclosed herein is administered in doses of about 175 mg. In some embodiments, the active pharmaceutical ingredient as disclosed herein is administered in doses of about 200 mg. In some embodiments, the active pharmaceutical ingredient as disclosed herein is administered in doses of about 225 mg. In some embodiments, the active pharmaceutical ingredient as disclosed herein is administered in doses of about 250 mg. In some embodiments, the active pharmaceutical ingredient as disclosed herein is administered in doses of about 275 mg. In some embodiments, the active pharmaceutical ingredient as disclosed herein is administered in doses of about 300 mg. In preferred embodiments, the active pharmaceutical ingredient is arimoclomol.

In some embodiments, the subject has a body weight of about 8 kg to about 15 kg and the active pharmaceutical ingredient as disclosed herein is administered at a dose of about 50 mg t.i.d. (i.e., about 150 mg/day). In some embodiments, the active pharmaceutical ingredient is arimoclomol (citrate salt).

In some embodiments, the subject has a body weight of about 8 kg and the active pharmaceutical ingredient as disclosed herein is administered at a dose of about 50 mg t.i.d. (i.e., about 150 mg/day). In some embodiments, the subject has a body weight of about 9 kg and the active pharmaceutical ingredient as disclosed herein is administered at a dose of about 50 mg t.i.d. (i.e., about 150 mg/day). In some embodiments, the subject has a body weight of about 10 kg and the active pharmaceutical ingredient as disclosed herein is administered at a dose of about 50 mg t.i.d. (i.e., about 150 mg/day). In some embodiments, the subject has a body weight of about 11 kg and the active pharmaceutical ingredient as disclosed herein is administered at a dose of about 50 mg t.i.d. (i.e., about 150 mg/day). In some embodiments, the subject has a body weight of about 12 kg and the active pharmaceutical ingredient as disclosed herein is administered at a dose of about 50 mg t.i.d. (i.e., about 150 mg/day). In some embodiments, the subject has a body weight of about 13 kg and the active pharmaceutical ingredient as disclosed herein is administered at a dose of about 50 mg t.i.d. (i.e., about 150 mg/day). In some embodiments, the subject has a body weight of about 14 kg and the active pharmaceutical ingredient as disclosed herein is administered at a dose of about 50 mg t.i.d. (i.e., about 150 mg/day). In some embodiments, the subject has a body weight of about 15 kg and the active pharmaceutical ingredient as disclosed herein is administered at a dose of about 50 mg t.i.d. (i.e., about 150 mg/day). In some embodiments, the active pharmaceutical ingredient is arimoclomol (citrate salt).

In some embodiments, the subject has a body weight of about 15 kg to about 22 kg and the active pharmaceutical ingredient as disclosed herein is administered at a dose of about 75 mg t.i.d. (i.e., about 225 mg/day). In some embodiments, the active pharmaceutical ingredient is arimoclomol (citrate salt).

In some embodiments, the subject has a body weight of about 15 kg and the active pharmaceutical ingredient as disclosed herein is administered at a dose of about 75 mg t.i.d. (i.e., about 225 mg/day). In some embodiments, the subject has a body weight of about 16 kg and the active pharmaceutical ingredient as disclosed herein is administered at a dose of about 75 mg t.i.d. (i.e., about 225 mg/day). In some embodiments, the subject has a body weight of about 17 kg and the active pharmaceutical ingredient as disclosed herein is administered at a dose of about 75 mg t.i.d. (i.e., about 225 mg/day). In some embodiments, the subject has a body weight of about 18 kg and the active pharmaceutical ingredient as disclosed herein is administered at a dose of about 75 mg t.i.d. (i.e., about 225 mg/day). In some embodiments, the subject has a body weight of about 19 kg and the active pharmaceutical ingredient as disclosed herein is administered at a dose of about 75 mg t.i.d. (i.e., about 225 mg/day). In some embodiments, the subject has a body weight of about 20 kg and the active pharmaceutical ingredient as disclosed herein is administered at a dose of about 75 mg t.i.d. (i.e., about 225 mg/day). In some embodiments, the subject has a body weight of about 21 kg and the active pharmaceutical ingredient as disclosed herein is administered at a dose of about 75 mg t.i.d. (i.e., about 225 mg/day). In some embodiments, the subject has a body weight of about 22 kg and the active pharmaceutical ingredient as disclosed herein is administered at a dose of about 75 mg t.i.d. (i.e., about 225 mg/day). In some embodiments, the active pharmaceutical ingredient is arimoclomol (citrate salt).

In some embodiments, the subject has a body weight of about 22 kg to about 38 kg and the active pharmaceutical ingredient as disclosed herein is administered at a dose of about 100 mg t.i.d. (i.e., about 300 mg/day). In some embodiments, the active pharmaceutical ingredient is arimoclomol (citrate salt).

In some embodiments, the subject has a body weight of about 22 kg and the active pharmaceutical ingredient as disclosed herein is administered at a dose of about 100 mg t.i.d. (i.e., about 300 mg/day). In some embodiments, the subject has a body weight of about 23 kg and the active pharmaceutical ingredient as disclosed herein is administered at a dose of about 100 mg t.i.d. (i.e., about 300 mg/day). In some embodiments, the subject has a body weight of about 24 kg and the active pharmaceutical ingredient as disclosed herein is administered at a dose of about 100 mg t.i.d. (i.e., about 300 mg/day). In some embodiments, the subject has a body weight of about 25 kg and the active pharmaceutical ingredient as disclosed herein is administered at a dose of about 100 mg t.i.d. (i.e., about 300 mg/day). In some embodiments, the subject has a body weight of about 26 kg and the active pharmaceutical ingredient as disclosed herein is administered at a dose of about 100 mg t.i.d. (i.e., about 300 mg/day). In some embodiments, the subject has a body weight of about 27 kg and the active pharmaceutical ingredient as disclosed herein is administered at a dose of about 100 mg t.i.d. (i.e., about 300 mg/day). In some embodiments, the subject has a body weight of about 28 kg and the active pharmaceutical ingredient as disclosed herein is administered at a dose of about 100 mg t.i.d. (i.e., about 300 mg/day). In some embodiments, the subject has a body weight of about 29 kg and the active pharmaceutical ingredient as disclosed herein is administered at a dose of about 100 mg t.i.d. (i.e., about 300 mg/day). In some embodiments, the subject has a body weight of about 30 kg and the active pharmaceutical ingredient as disclosed herein is administered at a dose of about 100 mg t.i.d. (i.e., about 300 mg/day). In some embodiments, the subject has a body weight of about 31 kg and the active pharmaceutical ingredient as disclosed herein is administered at a dose of about 100 mg t.i.d. (i.e., about 300 mg/day). In some embodiments, the subject has a body weight of about 32 kg and the active pharmaceutical ingredient as disclosed herein is administered at a dose of about 100 mg t.i.d. (i.e., about 300 mg/day). In some embodiments, the subject has a body weight of about 33 kg and the active pharmaceutical ingredient as disclosed herein is administered at a dose of about 100 mg t.i.d. (i.e., about 300 mg/day). In some embodiments, the subject has a body weight of about 34 kg and the active pharmaceutical ingredient as disclosed herein is administered at a dose of about 100 mg t.i.d. (i.e., about 300 mg/day). In some embodiments, the subject has a body weight of about 35 kg and the active pharmaceutical ingredient as disclosed herein is administered at a dose of about 100 mg t.i.d. (i.e., about 300 mg/day). In some embodiments, the subject has a body weight of about 36 kg and the active pharmaceutical ingredient as disclosed herein is administered at a dose of about 100 mg t.i.d. (i.e., about 300 mg/day). In some embodiments, the subject has a body weight of about 37 kg and the active pharmaceutical ingredient as disclosed herein is administered at a dose of about 100 mg t.i.d. (i.e., about 300 mg/day). In some embodiments, the subject has a body weight of about 38 kg and the active pharmaceutical ingredient as disclosed herein is administered at a dose of about 100 mg t.i.d. (i.e., about 300 mg/day). In some embodiments, the active pharmaceutical ingredient is arimoclomol (citrate salt).

In some embodiments, the subject has a body weight of about 38 kg to about 55 kg and the active pharmaceutical ingredient as disclosed herein is administered at a dose of about 150 mg t.i.d. (i.e., about 450 mg/day). In some embodiments, the active pharmaceutical ingredient is arimoclomol (citrate salt).

In some embodiments, the subject has a body weight of about 38 kg and the active pharmaceutical ingredient as disclosed herein is administered at a dose of about 150 mg t.i.d. (i.e., about 450 mg/day). In some embodiments, the subject has a body weight of about 39 kg and the active pharmaceutical ingredient as disclosed herein is administered at a dose of about 150 mg t.i.d. (i.e., about 450 mg/day). In some embodiments, the subject has a body weight of about 40 kg and the active pharmaceutical ingredient as disclosed herein is administered at a dose of about 150 mg t.i.d. (i.e., about 450 mg/day). In some embodiments, the subject has a body weight of about 41 kg and the active pharmaceutical ingredient as disclosed herein is administered at a dose of about 150 mg t.i.d. (i.e., about 450 mg/day). In some embodiments, the subject has a body weight of about 42 kg and the active pharmaceutical ingredient as disclosed herein is administered at a dose of about 150 mg t.i.d. (i.e., about 450 mg/day). In some embodiments, the subject has a body weight of about 43 kg and the active pharmaceutical ingredient as disclosed herein is administered at a dose of about 150 mg t.i.d. (i.e., about 450 mg/day). In some embodiments, the subject has a body weight of about 44 kg and the active pharmaceutical ingredient as disclosed herein is administered at a dose of about 150 mg t.i.d. (i.e., about 450 mg/day). In some embodiments, the subject has a body weight of about 45 kg and the active pharmaceutical ingredient as disclosed herein is administered at a dose of about 150 mg t.i.d. (i.e., about 450 mg/day). In some embodiments, the subject has a body weight of about 46 kg and the active pharmaceutical ingredient as disclosed herein is administered at a dose of about 150 mg t.i.d. (i.e., about 450 mg/day). In some embodiments, the subject has a body weight of about 47 kg and the active pharmaceutical ingredient as disclosed herein is administered at a dose of about 150 mg t.i.d. (i.e., about 450 mg/day). In some embodiments, the subject has a body weight of about 48 kg and the active pharmaceutical ingredient as disclosed herein is administered at a dose of about 150 mg t.i.d. (i.e., about 450 mg/day). In some embodiments, the subject has a body weight of about 49 kg and the active pharmaceutical ingredient as disclosed herein is administered at a dose of about 150 mg t.i.d. (i.e., about 450 mg/day). In some embodiments, the subject has a body weight of about 50 kg and the active pharmaceutical ingredient as disclosed herein is administered at a dose of about 150 mg t.i.d. (i.e., about 450 mg/day). In some embodiments, the subject has a body weight of about 51 kg and the active pharmaceutical ingredient as disclosed herein is administered at a dose of about 150 mg t.i.d. (i.e., about 450 mg/day). In some embodiments, the subject has a body weight of about 52 kg and the active pharmaceutical ingredient as disclosed herein is administered at a dose of about 150 mg t.i.d. (i.e., about 450 mg/day). In some embodiments, the subject has a body weight of about 53 kg and the active pharmaceutical ingredient as disclosed herein is administered at a dose of about 150 mg t.i.d. (i.e., about 450 mg/day). In some embodiments, the subject has a body weight of about 54 kg and the active pharmaceutical ingredient as disclosed herein is administered at a dose of about 150 mg t.i.d. (i.e., about 450 mg/day). In some embodiments, the subject has a body weight of about 55 kg and the active pharmaceutical ingredient as disclosed herein is administered at a dose of about 150 mg t.i.d. (i.e., about 450 mg/day). In some embodiments, the active pharmaceutical ingredient is arimoclomol (citrate salt).

In some embodiments, the subject has a body weight of greater than about 55 kg and the active pharmaceutical ingredient as disclosed herein is administered at a dose of about 200 mg t.i.d. (i.e., about 600 mg/day). In some embodiments, the active pharmaceutical ingredient is arimoclomol (citrate salt).

In some embodiments, the subject has a body weight of about 8 kg to about 15 kg and the active pharmaceutical ingredient as disclosed herein is administered at a dose of about 75 mg t.i.d. (i.e., about 225 mg/day). In some embodiments, the active pharmaceutical ingredient is arimoclomol (citrate salt).

In some embodiments, the subject has a body weight of about 15 kg to about 30 kg and the active pharmaceutical ingredient as disclosed herein is administered at a dose of about 100 mg t.i.d. (i.e., about 300 mg/day). In some embodiments, the active pharmaceutical ingredient is arimoclomol (citrate salt).

In some embodiments, the subject has a body weight of about 30 kg to about 55 kg and the active pharmaceutical ingredient as disclosed herein is administered at a dose of about 150 mg t.i.d. (i.e., about 450 mg/day). In some embodiments, the active pharmaceutical ingredient is arimoclomol (citrate salt).

In some embodiments, the subject has a body weight of greater than about 55 kg and the active pharmaceutical ingredient as disclosed herein is administered at a dose of about 200 mg t.i.d. (i.e., about 600 mg/day). In some embodiments, the active pharmaceutical ingredient is arimoclomol (citrate salt).

### Dosages, Arimoclomol (Free Base)

In some embodiments, the active ingredient is the arimoclomol free base form (no specific salt form), i.e. (+)-(R)-N-[2-hydroxy-3-(1-piperidinyl)-propoxy]-pyridine-1-oxide-3-carboximidoyl chloride, and dosages are calculated based on the free base form.

Reference to the arimoclomol free base form i.e. (+)-(R)-N-[2-hydroxy-3-(1-piperidinyl)-propoxy]-pyridine-1-oxide-3-carboximidoyl chloride, will encompass any acid addition salts thereof.

In some embodiments, the arimoclomol free base form is administered from about 53 mg/day to about 423 mg/day. In some embodiments, the arimoclomol free base form is administered from about 63 mg/day to about 423 mg/day. In some embodiments, the arimoclomol free base form is administered from about 73 mg/day to about 423 mg/day. In some embodiments, the arimoclomol free base form is administered from about 83 mg/day to about 423 mg/day. In some embodiments, the arimoclomol free base form is administered from about 93 mg/day to about 423 mg/day. In some embodiments, the arimoclomol free base form is administered from about 103 mg/day to about 423 mg/day. In some embodiments, the arimoclomol free base form is administered from about 113 mg/day to about 423 mg/day. In some embodiments, the arimoclomol free base form is administered from about 123 mg/day to about 423 mg/day. In some embodiments, the arimoclomol free base form is administered from about 133 mg/day to about 423 mg/day. In some embodiments, the arimoclomol free base form is administered from about 143 mg/day to about 423 mg/day. In some embodiments, the arimoclomol free base form is administered from about 153 mg/day to about 423 mg/day. In some embodiments, the arimoclomol free base form is administered from about 163 mg/day to about 423 mg/day. In some embodiments, the arimoclomol free base form is administered from about 173 mg/day to about 423 mg/day. In some embodiments, the arimoclomol free base form is administered from about 183 mg/day to about 423 mg/day. In some embodiments, the arimoclomol free base form is administered from about 193 mg/day to about 423 mg/day. In some embodiments, the arimoclomol free base form is administered from about 203 mg/day to about 423 mg/day. In some embodiments, the arimoclomol free base form is administered from about 213 mg/day to about 423 mg/day. In some embodiments, the arimoclomol free base form is administered from about 223 mg/day to about 423 mg/day. In some embodiments, the arimoclomol free base form is administered from about 243 mg/day to about 423 mg/day. In some embodiments, the arimoclomol free base form is administered from about 253 mg/day to about 423 mg/day. In some embodiments, the arimoclomol free base form is administered from about 263 mg/day to about 423 mg/day. In some embodiments, the arimoclomol free base form is administered from about 273 mg/day to about 423 mg/day. In some embodiments, the arimoclomol free base form is administered from about 283 mg/day to about 423 mg/day. In some embodiments, the arimoclomol free base form is administered from about 293 mg/day to about 423 mg/day. In some embodiments, the arimoclomol free base form is administered from about 303 mg/day to about 423 mg/day. In some embodiments, the arimoclomol free base form is administered from about 313 mg/day to about 423 mg/day. In some embodiments, the arimoclomol free base form is administered from about 323 mg/day to about 423 mg/day. In some embodiments, the arimoclomol free base form is administered from about 333 mg/day to about 423 mg/day. In some embodiments, the arimoclomol free base form is administered from about 343 mg/day to about 423 mg/day. In some embodiments, the arimoclomol free base form is administered from about 353 mg/day to about 423 mg/day. In some embodiments, the arimoclomol free base form is administered from about 363 mg/day to about 423 mg/day. In some embodiments, the arimoclomol free base form is administered from about 373 mg/day to about 423 mg/day. In some embodiments, the arimoclomol free base form is administered from about 383 mg/day to about 423 mg/day. In some embodiments, the arimoclomol free base form is administered from about 393 mg/day to about 423 mg/day. In some embodiments, the arimoclomol free base form is administered from about 403 mg/day to about 423 mg/day. In some embodiments, the arimoclomol free base form is administered from about 413 mg/day to about 423 mg/day.

In some embodiments, the arimoclomol free base form is administered from about 53 mg/day to about 413 mg/day. In some embodiments, the arimoclomol free base form is administered from about 53 mg/day to about 403 mg/day. In some embodiments, the arimoclomol free base form is administered from about 53 mg/day to about 393 mg/day. In some embodiments, the arimoclomol free base form is administered from about 53 mg/day to about 383 mg/day. In some embodiments, the arimoclomol free base form is administered from about 53 mg/day to about 373 mg/day. In some embodiments, the arimoclomol free base form is administered from about 53 mg/day to about 363 mg/day. In some embodiments, the arimoclomol free base form is administered from about 53 mg/day to about 353 mg/day. In some embodiments, the arimoclomol free base form is administered from about 53 mg/day to about 343 mg/day. In some embodiments, the arimoclomol free base form is administered from about 53 mg/day to about 333 mg/day. In some embodiments, the arimoclomol free base form is administered from about 53 mg/day to about 323 mg/day. In some embodiments, the arimoclomol free base form is administered from about 53 mg/day to about 313 mg/day. In some embodiments, the arimoclomol free base form is administered from about 53 mg/day to about 303 mg/day. In some embodiments, the arimoclomol free base form is administered from about 53 mg/day to about 293 mg/day. In some embodiments, the arimoclomol free base form is administered from about 53 mg/day to about 283 mg/day. In some embodiments, the arimoclomol free base form is administered from about 53 mg/day to about 273 mg/day. In some embodiments, the arimoclomol free base form is administered from about 53 mg/day to about 263 mg/day. In some embodiments, the arimoclomol free base form is administered from about 53 mg/day to about 253 mg/day. In some embodiments, the arimoclomol free base form is administered from about 53 mg/day to about 243 mg/day. In some embodiments, the arimoclomol free base form is administered from about 53 mg/day to about 233 mg/day. In some embodiments, the arimoclomol free base form is administered from about 53 mg/day to about 223 mg/day. In some embodiments, the arimoclomol free base form is administered from about 53 mg/day to about 213 mg/day. In some embodiments, the arimoclomol free base form is administered from about 53 mg/day to about 203 mg/day. In some embodiments, the arimoclomol free base form is administered from about 53 mg/day to about 193 mg/day. In some embodiments, the arimoclomol free base form is administered from about 53 mg/day to about 183 mg/day. In some embodiments, the arimoclomol free base form is administered from about 53 mg/day to about 173 mg/day. In some embodiments, the arimoclomol free base form is administered from about 53 mg/day to about 163 mg/day. In some embodiments, the arimoclomol free base form is administered from about 53 mg/day to about 153 mg/day. In some embodiments, the arimoclomol free base form is administered from about 53 mg/day to about 143 mg/day. In some embodiments, the arimoclomol free base form is administered from about 53 mg/day to about 133 mg/day. In some embodiments, the arimoclomol free base form is administered from about 53 mg/day to about 123 mg/day. In some embodiments, the arimoclomol free base form is administered from about 53 mg/day to about 113 mg/day. In some embodiments, the arimoclomol free base form is administered from about 53 mg/day to about 103 mg/day. In some embodiments, the arimoclomol free base form is administered from about 53 mg/day to about 93 mg/day. In some embodiments, the arimoclomol free base form is administered from about 53 mg/day to about 83 mg/day. In some embodiments, the arimoclomol free base form is administered from about 53 mg/day to about 73 mg/day. In some embodiments, the arimoclomol free base form is administered from about 53 mg/day to about 63 mg/day.

In some embodiments, the arimoclomol free base form is administered at about 53 mg/day, about 63 mg/day, about 73 mg/day, about 83 mg/day, about 93 mg/day, about 103 mg/day, about 113 mg/day, about 123 mg/day, about 133 mg/day, about 134 mg/day, about 143 mg/day, about 153 mg/day, about 163 mg/day, about 173 mg/day, about 183 mg/day, about 193 mg/day, about 203 mg/day, about 213 mg/day, about 223 mg/day, about 233 mg/day, about 243 mg/day, about 253 mg/day, about 263 mg/day, about 273 mg/day, about 283 mg/day, about 293 mg/day, about 303 mg/day, about 313 mg/day, about 323 mg/day, about 333 mg/day, about 343 mg/day, about 353 mg/day, about 363 mg/day, about 373 mg/day, about 383 mg/day, about 393 mg/day, about 403 mg/day, about 413 mg/day, or about 423 mg/day.

In some embodiments, the arimoclomol free base form is administered at about 53 mg/day. In some embodiments, the arimoclomol free base form is administered at about 63 mg/day. In some embodiments, the arimoclomol free base form is administered at about 73 mg/day. In some embodiments, the arimoclomol free base form is administered at about 83 mg/day. In some embodiments, the arimoclomol free base form is administered at about 93 mg/day. In some embodiments, the arimoclomol free base form is administered at about 103 mg/day. In some embodiments, the arimoclomol free base form is administered at about 113 mg/day. In some embodiments, the arimoclomol free base form is administered at about 123 mg/day. In some embodiments, the arimoclomol free base form is administered at about 133 mg/day. In some embodiments, the arimoclomol free base form is administered at about 134 mg/day. In some embodiments, the arimoclomol free base form is administered at about 143 mg/day. In some embodiments, the arimoclomol free base form is administered at about 153 mg/day. In some embodiments, the arimoclomol free base form is administered at about 163 mg/day. In some embodiments, the arimoclomol free base form is administered at about 173 mg/day. In some embodiments, the arimoclomol free base form is administered at about 183 mg/day. In some embodiments, the arimoclomol free base form is administered at about 193 mg/day. In some embodiments, the arimoclomol free base form is administered at about 203 mg/day. In some embodiments, the arimoclomol free base form is administered at about 213 mg/day. In some embodiments, the arimoclomol free base form is administered at about 223 mg/day. In some embodiments, the arimoclomol free base form is administered at about 233 mg/day. In some embodiments, the arimoclomol free base form is administered at about 243 mg/day. In some embodiments, the arimoclomol free base form is administered at about 253 mg/day. In some embodiments, the arimoclomol free base form is administered at about 263 mg/day. In some embodiments, the arimoclomol free base form is administered at about 273 mg/day. In some embodiments, the arimoclomol free base form is administered at about 283 mg/day. In some embodiments, the arimoclomol free base form is administered at about 293 mg/day. In some embodiments, the arimoclomol free base form is administered at about 303 mg/day. In some embodiments, the arimoclomol free base form is administered at about 313 mg/day. In some embodiments, the arimoclomol free base form is administered at about 323 mg/day. In some embodiments, the arimoclomol free base form is administered at about 333 mg/day. In some embodiments, the arimoclomol free base form is administered at about 343 mg/day. In some embodiments, the arimoclomol free base form is administered at about 353 mg/day. In some embodiments, the arimoclomol free base form is administered at about 363 mg/day. In some embodiments, the arimoclomol free base form is administered at about 373 mg/day. In some embodiments, the arimoclomol free base form is administered at about 383 mg/day. In some embodiments, the arimoclomol free base form is administered at about 393 mg/day. In some embodiments, the arimoclomol free base form is administered at about 403 mg/day. In some embodiments, the arimoclomol free base form is administered at about 413 mg/day. In some embodiments, the arimoclomol free base form is administered at about 423 mg/day.

In some embodiments, the arimoclomol free base form is administered at about 372 mg/day.

In some embodiments, the arimoclomol free base form is administered in doses of about 13 mg to about 143 mg. In some embodiments, the arimoclomol free base form is administered in doses of about 23 mg to about 143 mg. In some embodiments, the arimoclomol free base form is administered in doses of about 33 mg to about 143 mg. In some embodiments, the arimoclomol free base form is administered in doses of about 43 mg to about 143 mg. In some embodiments, the arimoclomol free base form is administered in doses of about 53 mg to about 143 mg. In some embodiments, the arimoclomol free base form is administered in doses of about 63 mg to about 143 mg. In some embodiments, the arimoclomol free base form is administered in doses of about 73 mg to about 143 mg. In some embodiments, the arimoclomol free base form is administered in doses of about 83 mg to about 143 mg. In some embodiments, the arimoclomol free base form is administered in doses of about 93 mg to about 143 mg. In some embodiments, the arimoclomol free base form is administered in doses of about 103 mg to about 143 mg. In some embodiments, the arimoclomol free base form is administered in doses of about 113 mg to about 143 mg. In some embodiments, the arimoclomol free base form is administered in doses of about 123 mg to about 143 mg. In some embodiments, the arimoclomol free base form is administered in doses of about 133 mg to about 143 mg.

In some embodiments, the arimoclomol free base form is administered in doses of about 13 mg to about 133 mg. In some embodiments, the arimoclomol free base form is administered in doses of about 13 mg to about 123 mg. In some embodiments, the arimoclomol free base form is administered in doses of about 13 mg to about 113 mg. In some embodiments, the arimoclomol free base form is administered in doses of about 13 mg to about 103 mg. In some embodiments, the arimoclomol free base form is administered in doses of about 13 mg to about 93 mg. In some embodiments, the arimoclomol free base form is administered in doses of about 13 mg to about 83 mg. In some embodiments, the arimoclomol free base form is administered in doses of about 13 mg to about 73 mg. In some embodiments, the arimoclomol free base form is administered in doses of about 13 mg to about 63 mg. In some embodiments, the arimoclomol free base form is administered in doses of about 13 mg to about 53 mg. In some embodiments, the arimoclomol free base form is administered in doses of about 13 mg to about 43 mg. In some embodiments, the arimoclomol free base form is administered in doses of about 13 mg to about 33 mg. In some embodiments, the arimoclomol free base form is administered in doses of about 13 mg to about 23 mg.

In some embodiments, the arimoclomol free base form is administered in doses of about 13 mg. In some embodiments, the arimoclomol free base form is administered in doses of about 23 mg. In some embodiments, the arimoclomol free base form is administered in doses of about 33 mg. In some embodiments, the arimoclomol free base form is administered in doses of about 43 mg. In some embodiments, the arimoclomol free base form is administered in doses of about 53 mg. In some embodiments, the arimoclomol free base form is administered in doses of about 63 mg. In some embodiments, the arimoclomol free base form is administered in doses of about 73 mg. In some embodiments, the arimoclomol free base form is administered in doses of about 83 mg. In some embodiments, the arimoclomol free base form is administered in doses of about 93 mg. In some embodiments, the arimoclomol free base form is administered in doses of about 103 mg. In some embodiments, the arimoclomol free base form is administered in doses of about 113 mg. In some embodiments, the arimoclomol free base form is administered in doses of about 123 mg. In some embodiments, the arimoclomol free base form is administered in doses of about 133 mg. In some embodiments, the arimoclomol free base form is administered in doses of about 143 mg.

In some embodiments, the arimoclomol free base form is administered in doses of about 124 mg.

In some embodiments, the active pharmaceutical ingredient as disclosed herein is administered at least one day a week. In some embodiments, the active pharmaceutical ingredient as disclosed herein is administered at least two days a week. In some embodiments, the active pharmaceutical ingredient as disclosed herein is administered at least three days a week. In some embodiments, the active pharmaceutical ingredient as disclosed herein is administered at least four days a week. In some embodiments, the active pharmaceutical ingredient as disclosed herein is administered at least five days a week. In some embodiments, the active pharmaceutical ingredient as disclosed herein is administered at least six days a week. In some embodiments, the active pharmaceutical ingredient as disclosed herein is administered at least seven days a week.

In some embodiments, the active pharmaceutical ingredient as disclosed herein is administered at least one time daily. In some embodiments, the active pharmaceutical ingredient as disclosed herein is administered at least two times daily. In some embodiments, the active pharmaceutical ingredient as disclosed herein is administered at least three times daily. In some embodiments, the active pharmaceutical ingredient as disclosed herein is administered at least four times daily. In some embodiments, the active pharmaceutical ingredient as disclosed herein is administered at least five times daily.

In some embodiments, the active pharmaceutical ingredient as disclosed herein is administered three times daily (t.i.d.).

In some embodiments, arimoclomol is administered at least one time daily. In some embodiments, arimoclomol is administered at least two times daily. In some embodiments, arimoclomol is administered at least three times daily. In some embodiments, arimoclomol is administered at least four times daily. In some embodiments, arimoclomol is administered at least five times daily.

In some embodiments, the arimoclomol free base form is administered at least one time daily. In some embodiments, the arimoclomol free base form is administered at least two times daily. In some embodiments, the arimoclomol free base form is administered at least three times daily. In some embodiments, the arimoclomol free base form is administered at least four times daily. In some embodiments, the arimoclomol free base form is administered at least five times daily.

In some embodiments, the arimoclomol free base form is administered three times daily at about 93 mg/day to about 372 mg/day.

In some embodiments, arimoclomol is administered at least one day a week. In some embodiments, arimoclomol is administered at least two days a week. In some embodiments, arimoclomol is administered at least three days a week. In some embodiments, arimoclomol is administered at least four days a week. In some embodiments, arimoclomol is administered at least five days a week. In some embodiments, arimoclomol is administered at least six days a week. In some embodiments, arimoclomol is administered at least seven days a week.

In some embodiments, the arimoclomol free base form is administered at least one day a week. In some embodiments, the arimoclomol free base form is administered at least two days a week. In some embodiments, the arimoclomol free base form is administered at least three days a week. In some embodiments, the arimoclomol free base form is administered at least four days a week. In some embodiments, the arimoclomol free base form is administered at least five days a week. In some embodiments, the arimoclomol free base form is administered at least six days a week. In some embodiments, the arimoclomol free base form is administered at least seven days a week.

In some embodiments, the active pharmaceutical ingredient as disclosed herein is administered in a dosage adjusted by patient body weight. Thus, in some embodiments, arimoclomol is administered in a dosage adjusted by patient body weight. Likewise, in some embodiments, the arimoclomol free base form is administered in a dosage adjusted by patient body weight.

In some embodiments, the subject has a body weight of about 8 kg to about 15 kg and the active pharmaceutical ingredient as disclosed herein is administered at a dose of about 31 mg t.i.d. (i.e., about 93 mg/day), calculated as arimoclomol free base.

In some embodiments, the subject has a body weight of about 15 kg to about 22 kg and the active pharmaceutical ingredient as disclosed herein is administered at a dose of about 47 mg t.i.d. (i.e., about 141 mg/day); calculated as arimoclomol free base.

In some embodiments, the subject has a body weight of about 22 kg to about 38 kg and the active pharmaceutical ingredient as disclosed herein is administered at a dose of about 62 mg t.i.d. (i.e., about 186 mg/day); calculated as arimoclomol free base.

In some embodiments, the subject has a body weight of about 38 kg to about 55 kg and the active pharmaceutical ingredient as disclosed herein is administered at a dose of about 93 mg t.i.d. (i.e., about 279 mg/day); calculated as arimoclomol free base.

In some embodiments, the subject has a body weight of greater than about 55 kg and the active pharmaceutical ingredient as disclosed herein is administered at a dose of about 124 mg t.i.d. (i.e., about 372 mg/day); calculated as arimoclomol free base.

In some embodiments, the subject has a body weight of about 8 kg to about 15 kg and the active pharmaceutical ingredient as disclosed herein is administered at a dose of about 47 mg t.i.d. (i.e., about 141 mg/day), calculated as arimoclomol free base.

In some embodiments, the subject has a body weight of about 15 kg to about 30 kg and the active pharmaceutical ingredient as disclosed herein is administered at a dose of about 62 mg t.i.d. (i.e., about 186 mg/day); calculated as arimoclomol free base.

In some embodiments, the subject has a body weight of about 30 kg to about 55 kg and the active pharmaceutical ingredient as disclosed herein is administered at a dose of about 93 mg t.i.d. (i.e., about 279 mg/day); calculated as arimoclomol free base.

In some embodiments, the subject has a body weight of greater than about 55 kg and the active pharmaceutical ingredient as disclosed herein is administered at a dose of about 124 mg t.i.d. (i.e., about 372 mg/day); calculated as arimoclomol free base.

### Dosage, Further Active Pharmaceutical Ingredient

In some embodiments, a further active pharmaceutical ingredient is administered to the subject. In a preferred embodiment, the further active pharmaceutical ingredient is miglustat.

In some embodiments, the further active pharmaceutical ingredient as disclosed herein is administered from about 300 mg/day to about 1000 mg/day. In some embodiments, the further active pharmaceutical ingredient as disclosed herein is administered from about 350 mg/day to about 1000 mg/day. In some embodiments, the further active pharmaceutical ingredient as disclosed herein is administered from about 400 mg/day to about 1000 mg/day. In some embodiments, the further active pharmaceutical ingredient as disclosed herein is administered from about 450 mg/day to about 1000 mg/day. In some embodiments, the further active pharmaceutical ingredient as disclosed herein is administered from about 500 mg/day to about 1000 mg/day. In some embodiments, the further active pharmaceutical ingredient as disclosed herein is administered from about 550 mg/day to about 1000 mg/day. In some embodiments, the further active pharmaceutical ingredient as disclosed herein is administered from about 600 mg/day to about 1000 mg/day. In some embodiments, the further active pharmaceutical ingredient as disclosed herein is administered from about 650 mg/day to about 1000 mg/day. In some embodiments, the further active pharmaceutical ingredient as disclosed herein is administered from about 700 mg/day to about 1000 mg/day. In some embodiments, the further active pharmaceutical ingredient as disclosed herein is administered from about 750 mg/day to about 1000 mg/day. In some embodiments, the further active pharmaceutical ingredient as disclosed herein is administered from about 800 mg/day to about 1000 mg/day. In some embodiments, the further active pharmaceutical ingredient as disclosed herein is administered from about 850 mg/day to about 1000 mg/day. In some embodiments, the further active pharmaceutical ingredient as disclosed herein is administered from about 900 mg/day to about 1000 mg/day. In some embodiments, the further active pharmaceutical ingredient as disclosed herein is administered from about 950 mg/day to about 1000 mg/day. In preferred embodiments, the further active pharmaceutical ingredient as disclosed herein is miglustat.

In some embodiments, the further active pharmaceutical ingredient as disclosed herein is administered from about 300 mg/day to about 950 mg/day. In some embodiments, the further active pharmaceutical ingredient as disclosed herein is administered from about 300 mg/day to about 900 mg/day. In some embodiments, the further active pharmaceutical ingredient as disclosed herein is administered from about 300 mg/day to about 850 mg/day. In some embodiments, the further active pharmaceutical ingredient as disclosed herein is administered from about 300 mg/day to about 800 mg/day. In some embodiments, the further active pharmaceutical ingredient as disclosed herein is administered from about 300 mg/day to about 750 mg/day. In some embodiments, the further active pharmaceutical ingredient as disclosed herein is administered from about 300 mg/day to about 700 mg/day. In some embodiments, the further active pharmaceutical ingredient as disclosed herein is administered from about 300 mg/day to about 650 mg/day. In some embodiments, the further active pharmaceutical ingredient as disclosed herein is administered from about 300 mg/day to about 600 mg/day. In some embodiments, the further active pharmaceutical ingredient as disclosed herein is administered from about 300 mg/day to about 550 mg/day. In some embodiments, the further active pharmaceutical ingredient as disclosed herein is administered from about 300 mg/day to about 500 mg/day. In some embodiments, the further active pharmaceutical ingredient as disclosed herein is administered from about 300 mg/day to about 450 mg/day. In some embodiments, the further active pharmaceutical ingredient as disclosed herein is administered from about 300 mg/day to about 400 mg/day. In some embodiments, the further active pharmaceutical ingredient as disclosed herein is administered from about 300 mg/day to about 350 mg/day. In preferred embodiments, the further active pharmaceutical ingredient as disclosed herein is miglustat.

In some embodiments, the further active pharmaceutical ingredient as disclosed herein is administered at about 300 mg/day. In some embodiments, the further active pharmaceutical ingredient as disclosed herein is administered at about 325 mg/day. In some embodiments, the further active pharmaceutical ingredient as disclosed herein is administered at about 350 mg/day. In some embodiments, the further active pharmaceutical ingredient as disclosed herein is administered at about 375 mg/day. In some embodiments, the further active pharmaceutical ingredient as disclosed herein is administered at about 400 mg/day. In some embodiments, the further active pharmaceutical ingredient as disclosed herein is administered at about 425 mg/day. In some embodiments, the further active pharmaceutical ingredient as disclosed herein is administered at about 450 mg/day. In some embodiments, the further active pharmaceutical ingredient as disclosed herein is administered at about 475 mg/day. In some embodiments, the further active pharmaceutical ingredient as disclosed herein is administered at about 500 mg/day. In some embodiments, the further active pharmaceutical ingredient as disclosed herein is administered at about 525 mg/day. In some embodiments, the further active pharmaceutical ingredient as disclosed herein is administered at about 550 mg/day. In some embodiments, the further active pharmaceutical ingredient as disclosed herein is administered at about 575 mg/day. In some embodiments, the further active pharmaceutical ingredient as disclosed herein is administered at about 600 mg/day. In some embodiments, the further active pharmaceutical ingredient as disclosed herein is administered at about 625 mg/day. In some embodiments, the further active pharmaceutical ingredient as disclosed herein is administered at about 650 mg/day. In some embodiments, the further active pharmaceutical ingredient as disclosed herein is administered at about 675 mg/day. In some embodiments, the further active pharmaceutical ingredient as disclosed herein is administered at about 700 mg/day. In some embodiments, the further active pharmaceutical ingredient as disclosed herein is administered at about 725 mg/day. In some embodiments, the further active pharmaceutical ingredient as disclosed herein is administered at about 750 mg/day. In some embodiments, the further active pharmaceutical ingredient as disclosed herein is administered at about 775 mg/day. In some embodiments, the further active pharmaceutical ingredient as disclosed herein is administered at about 800 mg/day. In some embodiments, the further active pharmaceutical ingredient as disclosed herein is administered at about 825 mg/day. In some embodiments, the further active pharmaceutical ingredient as disclosed herein is administered at about 850 mg/day. In some embodiments, the further active pharmaceutical ingredient as disclosed herein is administered at about 875 mg/day. In some embodiments, the further active pharmaceutical ingredient as disclosed herein is administered at about 900 mg/day. In some embodiments, the further active pharmaceutical ingredient as disclosed herein is administered at about 925 mg/day. In some embodiments, the further active pharmaceutical ingredient as disclosed herein is administered at about 950 mg/day. In some embodiments, the further active pharmaceutical ingredient as disclosed herein is administered at about 975 mg/day. In some embodiments, the further active pharmaceutical ingredient as disclosed herein is administered at about 1000 mg/day. In preferred embodiments, the further active pharmaceutical ingredient as disclosed herein is miglustat.

In some embodiments, the further active pharmaceutical ingredient as disclosed herein is administered in doses of about 100 mg to about 300 mg. In some embodiments, the further active pharmaceutical ingredient as disclosed herein is administered in doses of about 125 mg to about 300 mg. In some embodiments, the further active pharmaceutical ingredient as disclosed herein is administered in doses of about 150 mg to about 300 mg. In some embodiments, the further active pharmaceutical ingredient as disclosed herein is administered in doses of about 175 mg to about 300 mg. In some embodiments, the further active pharmaceutical ingredient as disclosed herein is administered in doses of about 200 mg to about 300 mg. In some embodiments, the further active pharmaceutical ingredient as disclosed herein is administered in doses of about 225 mg to about 300 mg. In some embodiments, the further active pharmaceutical ingredient as disclosed herein is administered in doses of about 250 mg to about 300 mg. In some embodiments, the further active pharmaceutical ingredient as disclosed herein is administered in doses of about 275 mg to about 300 mg. In preferred embodiments, the further active pharmaceutical ingredient as disclosed herein is miglustat.

In some embodiments, the further active pharmaceutical ingredient as disclosed herein is administered in doses of about 100 mg to about 275 mg. In some embodiments, the further active pharmaceutical ingredient as disclosed herein is administered in doses of about 100 mg to about 250 mg. In some embodiments, the further active pharmaceutical ingredient as disclosed herein is administered in doses of about 100 mg to about 225 mg. In some embodiments, the further active pharmaceutical ingredient as disclosed herein is administered in doses of about 100 mg to about 200 mg. In some embodiments, the further active pharmaceutical ingredient as disclosed herein is administered in doses of about 100 mg to about 175 mg. In some embodiments, the further active pharmaceutical ingredient as disclosed herein is administered in doses of about 100 mg to about 150 mg. In some embodiments, the further active pharmaceutical ingredient as disclosed herein is administered in doses of about 100 mg to about 125 mg. In preferred embodiments, the further active pharmaceutical ingredient as disclosed herein is miglustat.

In some embodiments, the further active pharmaceutical ingredient as disclosed herein is administered in doses of about 100 mg. In some embodiments, the further active pharmaceutical ingredient as disclosed herein is administered in doses of about 125 mg. In some embodiments, the further active pharmaceutical ingredient as disclosed herein is administered in doses of about 150 mg. In some embodiments, the further active pharmaceutical ingredient as disclosed herein is administered in doses of about 175 mg. In some embodiments, the further active pharmaceutical ingredient as disclosed herein is administered in doses of about 200 mg. In some embodiments, the further active pharmaceutical ingredient as disclosed herein is administered in doses of about 225 mg. In some embodiments, the further active pharmaceutical ingredient as disclosed herein is administered in doses of about 250 mg. In some embodiments, the further active pharmaceutical ingredient as disclosed herein is administered in doses of about 275 mg. In some embodiments, the further active pharmaceutical ingredient as disclosed herein is administered in doses of about 300 mg. In preferred embodiments, the further active pharmaceutical ingredient as disclosed herein is miglustat.

In some embodiments, the dosage of the further active pharmaceutical ingredient, such as miglustat, is adjusted for subjects under the age of 12 years on the basis of body surface area.

### Co-Administration

In some embodiments, the active pharmaceutical ingredient and the further active pharmaceutical ingredient are co-administered. In some embodiments, the active pharmaceutical ingredient and the further active pharmaceutical ingredient are administered in temporal proximity. In some embodiments, the further active pharmaceutical ingredient is administered prior to the active pharmaceutical ingredient. In some embodiments, the active pharmaceutical ingredient is administered prior to the further active pharmaceutical ingredient. In some embodiments, the active pharmaceutical ingredient and the further active pharmaceutical ingredient are administered simultaneously or sequentially. In some embodiments, the active pharmaceutical ingredient is arimoclomol ((+)-(R)-N-[2-hydroxy-3-(1-piperidinyl)-propoxy]-pyridine-1-oxide-3-carboximidoyl chloride citrate) and the further active pharmaceutical ingredient is miglustat. In some embodiments, the active pharmaceutical ingredient is (+)-(R)-N-[2-hydroxy-3-(1-piperidinyl)-propoxy]-pyridine-1-oxide-3-carboximidoyl chloride and the further active pharmaceutical ingredient is miglustat.

In some embodiments, the further active pharmaceutical ingredient is administered for at least six months prior to an initial administration of the active pharmaceutical ingredient. In some embodiments, the further active pharmaceutical ingredient is administered for at least one year prior to an initial administration of the active pharmaceutical ingredient. In some embodiments, the further active pharmaceutical ingredient is administered for at least two years prior to an initial administration of the active pharmaceutical ingredient. In some embodiments, the active pharmaceutical ingredient is arimoclomol ((+)-(R)-N-[2-hydroxy-3-(1-piperidinyl)-propoxy]-pyridine-1-oxide-3-carboximidoyl chloride citrate) and the further active pharmaceutical ingredient is miglustat. In some embodiments, the active pharmaceutical ingredient is (+)-(R)-N-[2-hydroxy-3-(1-piperidinyl)-propoxy]-pyridine-1-oxide-3-carboximidoyl chloride and the further active pharmaceutical ingredient is miglustat.

In some embodiments, the active pharmaceutical ingredient as disclosed herein is administered orally. In some embodiments, the active pharmaceutical ingredient as disclosed herein is administered via a feeding tube.

### Effects of Treatment

In some embodiments, administration of the active pharmaceutical ingredient as disclosed herein provides sustained benefit over a period of time. In some embodiments, the period of time is a six-month period. In some embodiments, the period of time is a one-year period. In some embodiments, the period of time is a one-year and six-month period. In some embodiments, the period of time is a two-year period. In some embodiments, the period of time is a two-year and six-month period. In some embodiments, the period of time is a three-year period. In some embodiments, the active pharmaceutical ingredient is arimoclomol.

In some embodiments, the disease course is modified by the treatment as disclosed herein.

In some embodiments, the administration of the active pharmaceutical ingredient reduces accumulation of unesterified cholesterol. In some embodiments, the reduction of unesterified cholesterol occurs in peripheral blood mononuclear cells (PBMCs). In some aspects, the administration of the active pharmaceutical ingredient reduces unesterified cholesterol such that any increase in unesterified cholesterol exhibited by the subject is no more than about 10,000 ng/mg protein, or no more than about 15,000 ng/mg protein, or no more than about 20,000 ng/mg protein, or more than about 25,000 ng/mg protein, or no more than about 30,000 ng/mg protein, or nor more than about 35,000 ng/mg protein, or no more than about 40,000 ng/mg protein. In some embodiments, the active pharmaceutical ingredient is arimoclomol ((+)-(R)-N-[2-hydroxy-3-(1-piperidinyl)-propoxy]-pyridine-1-oxide-3-carboximidoyl chloride citrate). In some embodiments, the active pharmaceutical ingredient is (+)-(R)-N-[2-hydroxy-3-(1-piperidinyl)-propoxy]-pyridine-1-oxide-3-carboximidoyl chloride.

In some embodiments, the administration of the active pharmaceutical ingredient reduces accumulation of serum cholestane-triol levels. In some embodiments, the administration of the active pharmaceutical ingredient reduces the accumulation of serum cholestane-triol levels such that there is a decrease of at least about 2.5 ng/ml, or a decrease of at least about 3.0 ng/ml, or a decrease of at least about 3.5 ng/ml, or a decrease of at least about 4.0 ng/ml, or a decrease of at least about 4.5 ng/ml, or a decrease of at least about 5.0 ng/ml, or a decrease of at least about 5.5 ng/ml, or a decrease of at least about 6.0 ng/ml, or a decrease of at least about 6.5 ng/ml, or a decrease of at least about 7.0 ng/ml, or a decrease of at least about 7.5 ng/ml, or a decrease of at least about 7.5 ng/ml, or a decrease of at least about 8.0 ng/ml, or a decrease of at least about 8.5 ng/ml, or a decrease of at least about 9.0 ng/ml, or a decrease of at least about 9.5 ng/ml, or a decrease of at least about 10.0 ng/ml. In some embodiments, the active pharmaceutical ingredient is arimoclomol ((+)-(R)-N-[2-hydroxy-3-(1-piperidinyl)-propoxy]-pyridine-1-oxide-3-carboximidoyl chloride citrate). In some embodiments, the active pharmaceutical ingredient is (+)-(R)-N-[2-hydroxy-3-(1-piperidinyl)-propoxy]-pyridine-1-oxide-3-carboximidoyl chloride.

In some embodiments, the administration of the active pharmaceutical ingredient modifies the course of NPC such that the subject who is administered the active pharmaceutical ingredient exhibits an increase in NPCCSS score that is no more than about 0.1, such as about 0.2, such as about 0.3, such as about 0.4, such as about 0.5, such as about 0.6, such as about 0.7, such as about 0.8, such as about 0.9, such as about 1.0, such as about 1.1, such as about 1.2, such as about 1.3, such as about 1.4, such as about 1.5, such as about 1.6, such as about 1.7, such as about 1.8, such as about 1.9, such as about 2.0 over the course of treatment. In some embodiments, the course of treatment can be a period of at least about 1 month, such as about 2 months, such as about 3 months, such as about 4 months, such as about 5 months, such as about 6 months, such as about 7 months, such as about 8 months, such as about 9 months, such as about 10 months, such as about 11 months, such as about 12 months, such as about 13 months, such as about 14 months, such as about 15 months, such as about 16 months, such as about 17 months, such as about 18 months, such as about 19 months, such as about 20 months, such as about 21 months, such as about 22 months, such as about 23 months, such as about 24 months. In some embodiments, the course of treatment can be a period greater than 24 months. The NPCCSS score can be a 5-domain NPCCSS score or a 17-domain NPCCSS score, as described herein. In some embodiments, the active pharmaceutical ingredient is arimoclomol ((+)-(R)-N-[2-hydroxy-3-(1-piperidinyl)-propoxy]-pyridine-1-oxide-3-carboximidoyl chloride citrate). In some embodiments, the active pharmaceutical ingredient is (+)-(R)-N-[2-hydroxy-3-(1-piperidinyl)-propoxy]-pyridine-1-oxide-3-carboximidoyl chloride.

In some embodiments, administration of the active pharmaceutical ingredient provides sustained benefit over a two-year period, wherein the sustained benefit is characterized by the subject exhibiting a 5-domain NPCCSS score increase of no more than 1 over the two-year period. In some embodiments, the active pharmaceutical ingredient is arimoclomol ((+)-(R)-N-[2-hydroxy-3-(1-piperidinyl)-propoxy]-pyridine-1-oxide-3-carboximidoyl chloride citrate). In some embodiments, the active pharmaceutical ingredient is (+)-(R)-N-[2-hydroxy-3-(1-piperidinyl)-propoxy]-pyridine-1-oxide-3-carboximidoyl chloride.

In some embodiments, treatment with the active pharmaceutical ingredient leads to an increase in correctly matured NPC protein. In some embodiments, treatment with the active pharmaceutical ingredient leads to an increase in correctly matured NPC1 protein. In some embodiments, treatment with the active pharmaceutical ingredient leads to an increase in correctly matured NPC2 protein. In some embodiments, the active pharmaceutical ingredient is arimoclomol ((+)-(R)-N-[2-hydroxy-3-(1-piperidinyl)-propoxy]-pyridine-1-oxide-3-carboximidoyl chloride citrate). In some embodiments, the active pharmaceutical ingredient is (+)-(R)-N-[2-hydroxy-3-(1-piperidinyl)-propoxy]-pyridine-1-oxide-3-carboximidoyl chloride.

### Methods of Predicting Responsiveness to Treatment or Identifying Subjects Likely to Respond to Treatment

It is also an aspect of the present disclosure to provide a method of predicting the responsiveness of a subject with Niemann Pick disease, type C (NPC) to treatment with arimoclomol, the method comprising:
a) determining if the subject has an ER type missense mutation in an NPC gene; and
b) predicting that the subject will respond to treatment with arimoclomol when the subject is determined to have an ER type missense mutation in an NPC gene.

In some aspects is provided a method of predicting the responsiveness of a subject with Niemann Pick disease, type C (NPC) to treatment with a combination of arimoclomol and miglustat, the method comprising:
a) determining if the subject has an ER type missense mutation in an NPC gene; and
b) predicting that the subject will respond to treatment with a combination of arimoclomol and miglustat when the subject is determined to have an ER type missense mutation in an NPC gene.

In some aspects is provided a method of identifying a subject with Niemann Pick disease, type C (NPC) who is likely to be responsive to treatment with arimoclomol, the method comprising:
a) determining if the subject has an ER type missense mutation in an NPC gene; and
b) identifying the subject as being likely to respond to treatment with arimoclomol when the subject is determined to have an ER type missense mutation in an NPC gene.

In some aspects is provided a method of identifying a subject with Niemann Pick disease, type C (NPC) who is likely to be responsive to treatment with a combination of arimoclomol and miglustat, the method comprising:
a) determining if the subject has an ER type missense mutation in an NPC gene; and
b) identifying the subject as being likely to respond to treatment with a combination of arimoclomol and miglustat when the subject is determined to have an ER type missense mutation in an NPC gene.

In some embodiments, the subject has an ER type missense mutation in the NPC1 gene. In some embodiments, the subject has an ER type missense mutation in the NPC2 gene.

In some embodiments, determining an NPC genotype (e.g. an NPC1 genotype and/or an NPC2 genotype) comprises sequencing the nucleic acid isolated from a biological sample from the subject. As would be appreciated by the person skilled in the art, an NPC genotype (e.g. an NPC1 genotype and/or an NPC2 genotype) can be determined using any genotyping method known in the art, such as DNA sequencing.

In some embodiments, the subject is identified as being homozygous for an ER type missense mutation. In some embodiments, the subject is identified as being heterozygous for an ER type missense mutation. In some embodiments, the subject is identified as having at least one ER type missense mutation in each of the two alleles of the NPC gene. In some embodiments, the subject is identified as having at least one ER type missense mutation in one of the two alleles of the NPC gene. In some embodiments, the subject is identified as being a compound heterozygote for an ER type missense mutation in the NPC gene.

In one embodiment the method further comprising one or more steps of administering arimoclomol. In another embodiment the method further comprising one or more steps of administering arimoclomol and miglustat.

### EXAMPLES

### Example 1—Multinational Trial of Arimoclomol for the Treatment of NPC

The following example describes a 12-month, prospective, randomized, double-blind, placebo-controlled, phase 2/3 multinational trial performed to test the use of arimoclomol for the treatment of Niemann-Pick disease, type C (NPC).

### Methods

Study Participants: the NPC1 genotypes of the subjects enrolled in the study are shown in Table 1. Patients enrolled in the trial were stratified by use of miglustat at baseline. Patients in both strata were randomized 2:1 to receive arimoclomol or placebo.

TABLE 1
NPC1 genotypes of subjects enrolled in study

| NPC1 Allele 1 | NPC1 Allele 2 | |
|---|---|---|
| Amino acid | Amino acid | NPC1 Genotype |
| V20sp | P1007A | Missense/Functional Null |
| Q991fs | P1007A | Missense/Functional Null |
| A927V | S1004P | Missense/Missense |
| S940L | P1007A | Missense/Missense |
| I1061T | E1188* | Missense/Functional Null |
| A926V | P1007A | Missense/Missense |
| R404W | M1001V | Missense/Missense |
| I1061T | A1151T | Missense/Missense |
| I1061T | Q119fs | Missense/Functional Null |
| I1061T | I962fs | Missense/Functional Null |
| S734I | K822fs | Missense/Functional Null |
| P1007L | P1007L | Missense/Missense |
| T1036M | I1061T | Missense/Missense |
| G765V | P1007A | Missense/Missense |
| I1061T | N1156S | Missense/Missense |
| I1061T | V1141G | Missense/Missense |
| E718D | P1007L | Missense/Missense |
| V1165M | R116* | Missense/Functional Null |
| R1186H | R1186H | Missense/Missense |
| N968S | R1186H | Missense/Missense |
| E451K | G992W | Missense/Missense |
| P1007R | T1205K | Missense/Missense |
| R934* | F1079L | Missense/Functional Null |
| A1108fs | A1108fs | Functional Null/Functional Null |
| N1156S | F1199sp2 | Missense/Functional Null |
| V1165M | F1199sp1 | Missense/Functional Null |
| R518W | H641fs | Missense/Functional Null |
| G248V | L773fs | Missense/Functional Null |
| P1007A | R1186H | Missense/Missense |
| A1054T | H897Q | Missense/Missense |
| P733fs | V1078I | Missense/Functional Null |
| H641fs | S954L | Missense/Functional Null |
| D508fs | I1061T | Missense/Functional Null |
| Q991fs | I1061T | Missense/Functional Null |
| L860* | Q991fs | Functional Null/Functional Null |
| G910S | G910S | Missense/Missense |
| P1007A | L1204fs | Missense/Functional Null |
| P1007A | L1204fs | Missense/Functional Null |
| G886V | R978C | Missense/Missense |
| G886V | R978C | Missense/Missense |
| S357L | S940L | Missense/Missense |
| R518W | G992W | Missense/Missense |
| S940L | S940L | Missense/Missense |
| R518Q | R518Q | Functional Null/Functional Null |
| Y677N | R1059Q | Missense/Missense |
| T1036A | V1155G | Missense/Missense |
| H1016L | I1061T | Missense/Missense |
| P981L | L1248fs | Missense/Functional Null |
| L472P | L472P | Missense/Missense |
| I1061T | A1192fs | Missense/Functional Null |

Study Procedures: the screening visit (visit 1) included a baseline assessment, randomization assignment, and pharmacokinetic (PK) assessment. The first dose of arimoclomol or placebo was given within 1 week of randomization. Safety assessments were performed at visit 2 (7-14 days after start of treatment) and then every 3 months during the blinded period (visits 3-6). Monthly telephone follow-ups were performed to evaluate safety, confirm patients' weights, and assess treatment compliance.

Routine clinical care was maintained throughout the trial (including administration of miglustat). Each participant was randomized to receive arimoclomol or placebo three times daily. Arimoclomol (calculated as free base) was administered orally or by feeding tube at 93-372 mg/day based on the patient's body weight up to the estimated equivalence of 372 mg/day for adults (body weight>55 kg) or of 124 mg three times a day.

During the 12-month treatment phase, efficacy assessments for the primary endpoint, the 5-domain NPCCSS score, and for the non-disease specific Clinical Global Impression-Improvement scale (CGI-I) scores were performed at baseline and after 3, 6, 9, and 12 months of treatment; all other efficacy and biomarker assessments were performed at baseline and after 6 and 12 months of treatment.

### Outcomes

Primary endpoint: The primary endpoint was change from baseline in NPC severity at 12 months as assessed by the 5-domain NPCCSS score, an abbreviated assessment tool originating from the 17 domain NPCCSS score developed by Yanjanin et al., as described herein. The fully validated 5-domain NPCCSS score comprises the domains determined to be most clinically relevant to patients, caregivers, and clinicians: ambulation, cognition, fine motor skills, speech, and swallowing. The total aggregated 5-domain NPCCSS score ranges from 0 to 25, with a higher score indicating more severe clinical impairment.

Subgroup analyses of primary endpoint: NPC is a heterogeneous disease, and patients aged 2 to 18 years present with a large spectrum of disease presentations. The group of children<4 years old includes patients with mild manifestations and patients with aggressive, early fatal disease. As this group is particularly heterogeneous, a subgroup of patients ≥4 years of age was predefined with the population of study participants.

In the EU, miglustat is indicated for the treatment of progressive neurological manifestations in patients with NPC. However, not all patients are candidates for miglustat treatment, and it is recommended that the benefit of treatment should be evaluated on a regular basis (e.g., every six months). An analysis of the subpopulation of patients receiving miglustat was prespecified to elucidate the effect of arimoclomol in patients on background miglustat treatment. Overall, the subgroups of patients ≥4 years of age, and patients on miglustat treatment, were expected to be more homogeneous with respect to baseline demographics and disease characteristics and therefore more suitable for comparison.

Two post hoc subgroup analyses were also conducted.

In the first post hoc analysis, subjects with double functional null mutations in NPC1 (NPC1 genotype of Functional Null/Functional Null) were excluded.

In the second post hoc analysis, only subjects with annual severity increment score (ASIS) 0.5-2.0 were included. Cortina-Borja et al. suggests that by applying differential ASIS cut-off points of 0.5-2.0, which excludes the very mild and very severe patients, trial cohorts may be stratified to obtain a more homogeneous patient population that, if untreated, would change in clinical score within the typical period of a clinical trial.

In an additional post hoc analysis, only subjects with at least one ER type mutation were included.

Secondary endpoints: A secondary endpoint was responder analysis of Clinical Global Impression-Improvement scale (CGI-I) scores (responder defined as stable or improved) at 12 months compared with baseline. Other secondary endpoints were: responder analysis of 5-domain NPCCSS score (defined as stable or improved) at month 12 versus baseline; time to worsening on 5-domain NPCCSS score (defined as the time until the patient worsened by 2 points vs baseline); proportion of patients worsening on 5-domain NPCCSS score at 6 and 12 months by 2 points on the 5-domain NPCCSS score; and change in 17-domain NPCCSS score (excluding hearing domains) at 12 months. Additional secondary endpoints included: change from baseline in scale for assessment and rating of ataxia (SARA) score at 6 and 12 months; change in the nine-hole peg test (9-HPT) result at 6 and 12 months; change in health-related quality of life (HRQoL) as measured by the 5-dimension 3-level EuroQol questionnaire, youth version (EQ-5D-3L Y proxy) at 6 and 12 months; change in individual 5-domain NPCCSS score at month 12; and NPC clinical database (NPC-cdb) score changes from baseline at trial time points. The NPC-cdb score aims to reflect clinical status; an increase in score indicates a reduction in the patient's abilities. The score was calculated as defined by Stampfer et al.

The skilled person would readily appreciate that the SARA score, the 9-HPT, the HRQoL and the NPC-cdb are standardized, widely used clinical analysis techniques used to determine NPC progression in subjects.

CGI-I: the CGI-I was originally developed as a research rating tool to assess psychiatric diseases (see Busner and Targum, 2007). It provides the clinician's impression of improvement (or worsening) of a person's condition at the current visit compared with baseline on a 7-point scale ranging from 1 (very much improved) to 7 (very much worse). In the present trial, CGI-I was performed after clinical examination and patient interviews; the same investigator was instructed to perform CGI-I assessments throughout the trial for a given patient.

SARA: The SARA includes eight items reflecting neurological manifestations of cerebellar ataxia and provides a direct and simple description of motor function. The total score of the eight items ranges from 0 (normal) to 40 (not able to perform any of the test items) (see Schmitz-Hubsch et al.)

9-HPT: The 9-HPT is a direct and simple measurement of fine motor coordination, hand/eye coordination, and the ability to follow a simple direction, measured in seconds for each hand. The test is not applicable for children under 4 years of age (see Poole et al.)

EQ-5D-3L Y proxy: The HRQoL of individuals in the trial was measured using the child-friendly version of the EQ-5D-3L Y proxy questionnaire. The questionnaire was completed by the patient's caregiver as a proxy for the individual.

Quantification of Biomarkers: Cholestane-triol in serum, unesterified cholesterol, and HSP70 levels in peripheral blood mononuclear cells (PBMCs) were measured as described in Mengel et al.

### Results

The 50 subjects in the study were randomized (26 females; 24 males) from 14 sites in nine countries. Thirty-four patients received arimoclomol and 16 received placebo.

The proportion of patients completing 12 months of randomized treatment was 79.4% in the arimoclomol group and 93.8% in the placebo group. In the arimoclomol group, reasons for withdrawal included adverse events (n=3), withdrawal of consent (n=1), fast disease progression (early escape clause; n=2), and death from NPC progression (n=1). In the placebo group, one patient withdrew after one day owing to worsening of epilepsy (considered part of disease progression).

Baseline disease characteristics and demographics of patients are described in Table 2.

TABLE 2
Baseline characteristics and demographics of subjects in study

| | Arimoclomol | | Placebo | | Total | |
|---|---|---|---|---|---|---|
| | (n = 34) | | (n = 16) | | (n = 50) | |
| Age, years (mean [SD]) | 11.5 | (5.4) | 10.2 | (4.1) | 11.1 | (5.0) |
| Sex | | | | | | |
| Male | 17 | (50.0) | 7 | (43.8) | 24 | (48.0) |
| Female | 17 | (50.0) | 9 | (56.3) | 26 | (52.0) |
| Race | | | | | | |
| White | 32 | (94.1) | 13 | (81.3) | 45 | (90.0) |
| Asian | 1 | (2.9) | 1 | (6.3) | 2 | (4.0) |
| Native Hawaiian or other | 0 | | 1 | (6.3) | 1 | (2.0) |
| Pacific Islander | | | | | | |
| Other | 1 | (2.9) | 1 | (6.3) | 2 | (4.0) |
| BMI (kg/m²) | 18.72 | (4.15) | 19.46 | (3.33) | 18.95 | (3.89) |
| Age at diagnosis of first | 5.05 | (3.43) | 5.22 | (3.87) | 5.10 | (3.54) |
| neurological symptom (years), | | | | | | |
| mean (SD) | | | | | | |
| Age at first NPC symptom | | | | | | |
| (years), n (%) | | | | | | |
| Pre/peri-natal (<3 months) | 1 | (2.9) | 0 | | 1 | (2.0) |
| Early-infantile (3 months | 5 | (14.7) | 3 | (18.8) | 8 | (16.0) |
| to <2 years) | | | | | | |
| Late-infantile (2 to <6 years) | 17 | (50.0) | 7 | (43.8) | 24 | (48.0) |
| Juvenile (6 to 15 years) | 11 | (32.4) | 6 | (37.5) | 17 | (34.0) |
| Adolescent/adult (>15 years) | 0 | | 0 | | 0 | |
| Time since first NPC symptom | | | | | | |
| (years) | | | | | | |
| Mean (SD) | 7.61 | (4.54) | 8.07 | (3.75) | 7.76 | (4.27) |
| Median | 6.15 | | 8.10 | | 7.00 | |
| Range | 0.4-16.6 | | 2.0-14.8 | | 0.4-16.6 | |
| Time since NPC diagnosis | | | | | | |
| (years) | | | | | | |
| Mean (SD) | 5.59 | (4.36) | 5.11 | (4.14) | 5.43 | (4.25) |
| Median | 4.10 | | 3.00 | | 3.90 | |
| Range | 0.1-15.1 | | 0.8-14.2 | | 0.1-15.1 | |
| Treated with miglustat | | | | | | |
| Yes | 26 | (76.5) | 13 | (81.3) | 39 | (78.0) |
| History of seizure or epilepsy, | 12 | (35.3) | 2 | (12.5) | 14 | (28.0) |
| n (%) | | | | | | |
| Baseline 5-domain NPCCSS | | | | | | |
| score | | | | | | |
| Mean (SD) | 12.1 | (6.9) | 9.4 | (6.4) | 11.2 | (6.8) |
| Median | 11.5 | | 8.0 | | 10.5 | |
| Range | 1.0-24.0 | | 0.0-24.0 | | 0.0-24.0 | |
| Baseline 5-domain NPCCSS | | | | | | |
| score; individual domain | | | | | | |
| scores | | | | | | |
| Ambulation score; mean (SD) | 2.5 | (1.6) | 2.2 | (1.6) | 2.4 | (1.6) |
| Speech score; mean (SD) | 2.1 | (1.6) | 1.6 | (1.2) | 2.0 | (1.5) |
| Swallow score; mean (SD) | 1.9 | (1.7) | 1.3 | (1.7) | 1.7 | (1.7) |
| Fine motor skills score; | 2.8 | (1.8) | 1.9 | (1.8) | 2.5 | (1.9) |
| mean (SD) | | | | | | |
| Cognition score; mean (SD) | 2.8 | (1.3) | 2.5 | (1.5) | 2.7 | (1.3) |
| Baseline full-scale NPCCSS | 21.1 | (11.5) | 17.2 | (11.3) | 19.8 | (11.5) |
| score, except hearing | | | | | | |
| domains, mean (SD) | | | | | | |
| Baseline NPC-cdb, mean (SD) | 46.5 | (24.0) | 39.2 | (28.6) | 44.1 | (25.6) |

All subjects had a diagnosis of NPC with mutations in both NPC1 alleles. Most patients (n=39/50) were receiving miglustat as part of routine clinical care. Baseline mean 5-domain NPCCSS score, 17-domain NPCCSS score (excluding hearing domains), and NPC-cdb scores were higher in the arimoclomol group than in the placebo group (see Table 3). Subgroup baseline characteristics are summarized in Table 3.

TABLE 3
Baseline disease characteristics and demographics
by subgroup (full analysis set per subgroup).

| | Arimoclomol | | Placebo | | Total | |
|---|---|---|---|---|---|---|
| Receiving concomitant | 26 | | 13 | | 39 | |
| miglustat | | | | | | |
| Age (years) | | | | | | |
| Mean (SD) | 12.8 | (4.7) | 9.1 | (3.6) | 11.6 | (4.7) |
| Baseline 5-domain NPCCSS | | | | | | |
| score | | | | | | |
| Mean (SD) | 11.7 | (7.2) | 9.6 | (7.1) | 11.0 | (7.1) |
| Median (range) | 10.5 | (1.0-24.0) | 8.0 | (0-24.0) | 10.0 | (0-24.0) |
| Age at first neurological | | | | | | |
| symptom (years) | | | | | | |
| Mean (SD) | 5.25 | (3.34) | 4.04 | (3.20) | 4.85 | (3.31) |
| Patients with double functional | 0 | | 0 | | 0 | |
| null mutation (Functional | | | | | | |
| Null/Functional Null), n | | | | | | |
| Not receiving concomitant | 8 | | 3 | | 11 | |
| miglustat, n | | | | | | |
| Age (years) | | | | | | |
| Mean (SD) | 7.0 | (5.4) | 15.0 | (1.7) | 9.2 | (5.9) |
| Baseline 5-domain NPCCSS | | | | | | |
| score | | | | | | |
| Mean (SD) | 13.3 | (6.1) | 8.7 | (2.1) | 12.0 | (5.6) |
| Median (range) | 14.0 | (2.0-20.0) | 8.0 | (7.0-11.0) | 11.0 | (2.0-20.0) |
| Age at first neurological | | | | | | |
| symptom (years) | | | | | | |
| Mean (SD) | 4.39 | (3.87) | 10.33 | (1.53) | 6.01 | (4.32) |
| Patients with double functional | 3 | | 0 | | 3 | |
| null mutation, n | | | | | | |
| Age ≥4 years, n | 30 | | 14 | | 44 | |
| Age (years) | | | | | | |
| Mean (SD) | 12.7 | (4.5) | 11.2 | (3.2) | 12.2 | (4.2) |
| Baseline 5-domain NPCCSS | | | | | | |
| score | | | | | | |
| Mean (SD) | 12.0 | (6.9) | 10.3 | (6.4) | 12.2 | (4.2) |
| Median (range) | 13.5 | (4.0-19.0) | 11.0 | (7.0-16.0) | 12.5 | (4.0-19.0) |
| Age at first neurological | | | | | | |
| symptom (years) | | | | | | |
| Mean (SD) | 5.57 | (3.29) | 5.74 | (3.86) | 5.62 | (3.44) |
| Patients with double functional | 0 | | 0 | | 0 | |
| null mutation, n | | | | | | |
| Age <4 years, n | 4 | | 2 | | 6 | |
| Age (years) | | | | | | |
| Mean (SD) | 2.5 | (0.6) | 3.0 | (0.0) | 2.7 | (0.5) |
| Baseline 5-domain NPCCSS | | | | | | |
| score | | | | | | |
| Mean (SD) | 12.5 | (7.9) | 3.5 | (0.7) | 9.5 | (7.7) |
| Median (range) | 14.5 | (2.0-19.0) | 3.5 | (3.0-4.0) | 7.5 | (2.0-19.0) |
| Age at first neurological | | | | | | |
| symptom (years) | | | | | | |
| Mean (SD) | 1.13 | (1.30) | 1.58 | (0.59) | 1.28 | (1.07) |

Three patients, all randomized to the arimoclomol group, had double functional null mutations (see Table 4).

TABLE 4
Genotype analysis of NPC1 mutations of enrolled subjects

| | Arimoclomol | Placebo | | Total |
|---|---|---|---|---|
| Patient genotypes by | (n = 34 | (n = 16 | | (n = 50 |
| mutation type, n (%) | subjects) | subjects) | | subjects) |
| Double functional | 3 (8.8) | 0 | (0) | 3 (6.0) |
| null | | | | |
| Double missense | 16 (47.1) | 11 | (68.8) | 27 (54.0) |
| Missense/functional | 15 (44.1) | 5 | (31.2) | 20 (40.0) |
| null | | | | |

| Frequency of protein | Arimoclomol | Placebo | | Total |
|---|---|---|---|---|
| mutation types, n | (n = 68 | (n = 32 | | (n = 100 |
| alleles (%) | alleles) | alleles) | | alleles) |
| Missense | 47 (69.1) | 27 | (84.4) | 74 (74.0) |
| Functional null | 21 (30.9) | 5 | (15.6) | 26 (26.0) |
| Frameshift | 12 (17.6) | 5 | (15.6) | 17 (17.0) |
| Splicing | 5 (7.4) | 0 | (0) | 5 (5.0) |
| Premature stop | 4 (5.9) | 0 | (0) | 4 (4.0) |

| Characteristics of | Type | | Genotype |
|---|---|---|---|
| double functional | Arimoclomol | Placebo | Total |
| null mutations | (n = 34 | (n = 16 | (n = 50 |
| (arimoclomol group) | subjects) | subjects) | subjects) |

| | | |
|---|---|---|
| Patient 1 | Frameshift/frameshift | A1108fs/A1108fs |
| Patient 2 | Premature stop/frameshift | L860*/Q991fs |
| Patient 3 | Splicing/splicing | R518Q/R518Q |

For missense mutations in NPC1, the most common cellular phenotype is retention of misfolded NPC1 protein in the ER such that the protein doesn't reach the lysosome. The I1061T is the archetype of such ER mutations, and in vitro studies have shown that arimoclomol can increase the amount of correctly processed I1061T NPC1 in patient cells (see Example 2). Recent studies have revealed a number of additional mutations with the same cellular phenotype, such as C113R, R389L, G535V, L724P, Q921P, W942C, G1034C, V378A, R404Q, H510P, Q775P, M1142T, N1156S, G1162V, R1186H, L1244P, and I1061T (Shammas et al. 2019, Wang et al. 2020). Two of these were also found in this study: N1156S and R1186H (see Table 5). The ER type subgroup included 11 patients in the arimoclomol treatment group and 4 patients in the placebo group.

TABLE 5
Patients with at least one ER type missense mutation

| Patient | | | |
|---|---|---|---|
| ID | Genotype | Type | Treatment |
| 0108 | I1061T/E1188* | Missense/Functional Null | Arimoclomol |
| 0111 | I1061T/A1151T | Double Missense | Arimoclomol |
| 0202 | I1061T/Q119fs | Missense/Functional Null | Arimoclomol |
| 0206 | I1061T/I962fs | Missense/Functional Null | Arimoclomol |
| 0303 | T1036M/I1061T | Double Missense | Arimoclomol |
| 0307 | I1061T/V1141G | Double Missense | Arimoclomol |
| 0603 | N968S/R1186H | Double Missense | Arimoclomol |
| 1302 | N1156S/F1199sp2 | Missense/Functional Null | Arimoclomol |
| 1409 | Q991fs/I1061T | Missense/Functional Null | Arimoclomol |
| 2101 | H1016L/I1061T | Double Missense | Arimoclomol |
| 2303 | I1061T/A1192fs | Missense/Functional Null | Arimoclomol |
| 0305 | I1061T/N1156S | Double Missense | Placebo |
| 0602 | R1186H/R1186H | Double Missense | Placebo |
| 1404 | P1007A/R1186H | Double Missense | Placebo |
| 1408 | I1061T/D508fs | Missense/Functional Null | Placebo |

It is important to note that knowledge is limited regarding the folding status of most NPC1 missense mutations. Thus, the ER mutation list included here does not constitute a complete list of mutations amenable to a treatment that would increase the folding and maturation capacity of cells towards the NPC1 protein. Indeed, studies indicate that the majority of missense mutations in NPC have a misfolding component to their aetiology.

With the aim of assessing arimoclomol treatment effect in a genetically homogeneous subgroup, patients with at least one known ER missense mutations were identified. Demographic characteristics for the ER subgroup are shown in Table 6.

TABLE 6
Demographics of patients with ER type missense genotype

| | Arimoclomol | Placebo | Total |
|---|---|---|---|
| Full analysis set (N) | 11 | 4 | 15 |

Age (years)

| N | 11 | | 4 | | 15 | |
|---|---|---|---|---|---|---|
| Mean (SD) | 12.7 | (4.5) | 13.3 | (3.8) | 12.9 | (4.2) |

| | | | |
|---|---|---|---|
| Median | 12.0 | 14.5 | 13.0 |
| q25-q75 | 8.0-17.0 | 10.5-16.0 | 8.0-16.0 |
| Min-Max | 7-19 | 8-16 | 7-19 |

Sex (N, %)

| | | | | | | |
|---|---|---|---|---|---|---|
| Female | 6 | (54.5) | 1 | (25.0) | 7 | (46.7) |
| Male | 5 | (45.5) | 3 | (75.0) | 8 | (53.3) |
| Total | 11 | (100.0) | 4 | (100.0) | 15 | (100.0) |

Race (N, %)

| | | | | | | |
|---|---|---|---|---|---|---|
| White | 11 | (100.0) | 3 | (75.0) | 14 | (93.3) |
| Unknown | 1 | (25.0) | 1 | (6.7) | | |
| Total | 11 | (100.0) | 4 | (100.0) | 15 | (100.0) |

Weight (kg)

| N | 11 | | 4 | | 15 | |
|---|---|---|---|---|---|---|
| Mean (SD) | 44.2 | (13.0) | 45.9 | (21.7) | 44.7 | (14.9) |

| | | | |
|---|---|---|---|
| Median | 47.9 | 45.8 | 47.9 |
| q25-q75 | 32.1-55.5 | 30.1-61.8 | 32.1-55.5 |
| Min-Max | 20-60 | 20-72 | 20-72 |

Height (cm)

| N | 11 | | 4 | | 15 | |
|---|---|---|---|---|---|---|
| Mean (SD) | 150.0 | (20.8) | 152.0 | (23.1) | 150.5 | (20.6) |

| | | | |
|---|---|---|---|
| Median | 154.0 | 154.0 | 154.0 |
| q25-q75 | 130.0-168.0 | 134.5-169.5 | 130.0-168.0 |
| Min-Max | 117-179 | 123-177 | 117-179 |

BMI (kg/m2)

| N | 11 | | 4 | | 15 | |
|---|---|---|---|---|---|---|
| Mean (SD) | 19.20 | (2.33) | 18.67 | (4.02) | 19.06 | (2.72) |

| | | | |
|---|---|---|---|
| Median | 19.17 | 19.21 | 19.17 |
| q25-q75 | 17.89-20.22 | 16.03-21.32 | 17.89-20.22 |
| Min-Max | 14.5-23.0 | 13.3-23.0 | 13.3-23.0 |

Age at first neurological symptom (years)

| N | 11 | | 4 | | 15 | |
|---|---|---|---|---|---|---|
| Mean (SD) | 5.33 | (4.01) | 8.08 | (4.11) | 6.07 | (4.08) |

| | | | |
|---|---|---|---|
| Median | 4.00 | 8.50 | 6.00 |
| q25-q75 | 3.00-7.00 | 4.67-11.50 | 3.00-10.00 |
| Min-Max | 0.3-14.2 | 3.3-12.0 | 0.3-14.2 |

NPCCSS 5-Domain

| N | 11 | | 4 | | 15 | |
|---|---|---|---|---|---|---|
| Mean (SD) | 10.7 | (5.4) | 6.5 | (4.9) | 9.6 | (5.4) |

| | | | |
|---|---|---|---|
| Median | 8.0 | 7.0 | 8.0 |
| q25-q75 | 7.0-15.0 | 3.5-9.5 | 7.0-13.0 |
| Min-Max | 4-20 | 0-12 | 0-20 |

NPCCSS full scale except hearing domains

| N | 11 | | 4 | | 15 | |
|---|---|---|---|---|---|---|
| Mean (SD) | 19.7 | (9.5) | 13.0 | (9.0) | 17.9 | (9.6) |

| | | | |
|---|---|---|---|
| Median | 15.0 | 13.0 | 15.0 |
| q25-q75 | 10.0-30.0 | 7.5-18.5 | 10.0-28.0 |
| Min-Max | 9-33 | 2-24 | 2-33 |

N: Number of patients,
%: Percentage of patients,
SD: Standard deviation,
BMI: Body mass index
Demographics are measured at baseline

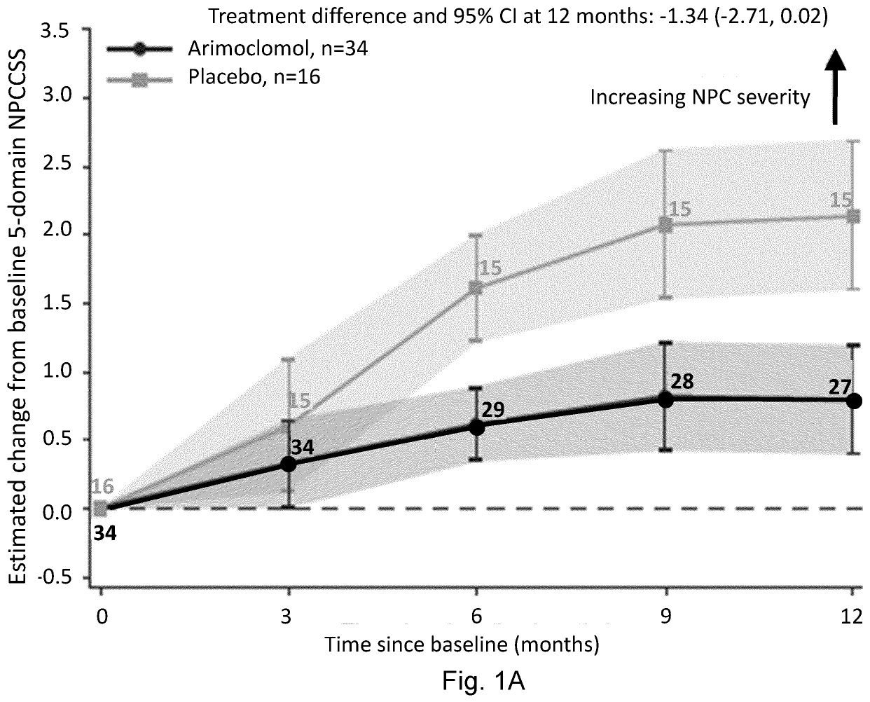

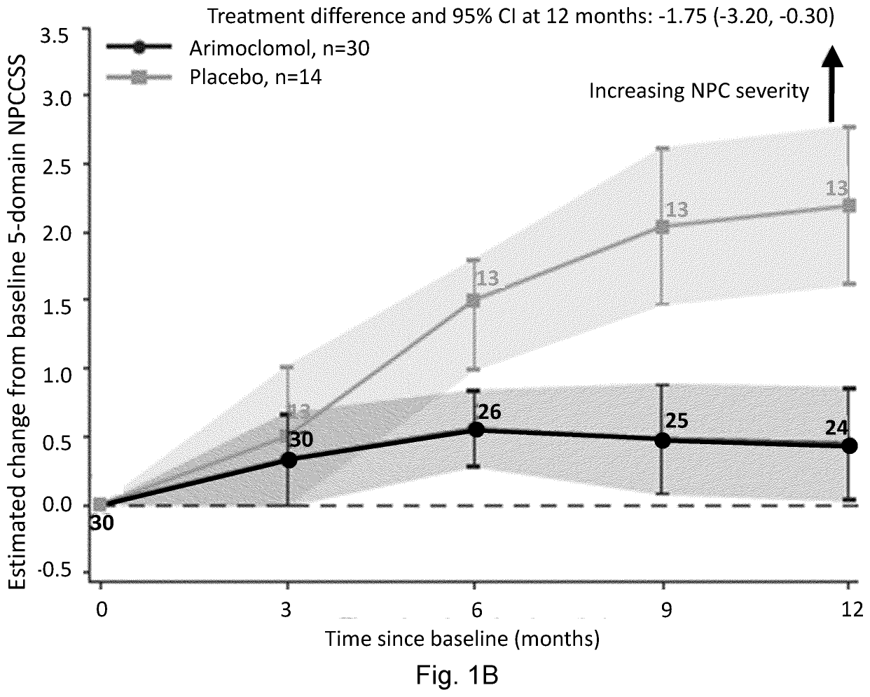

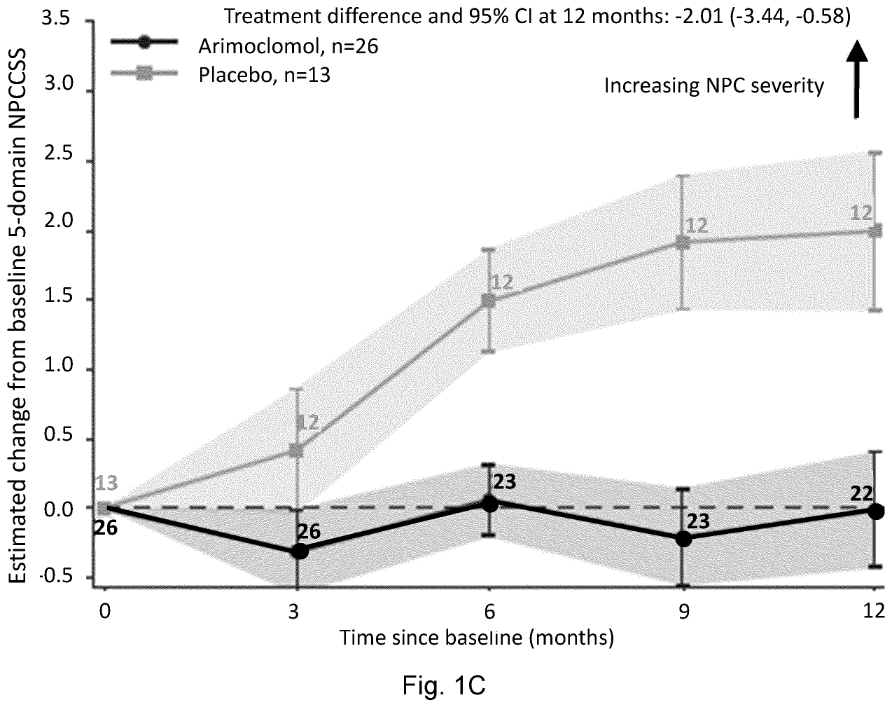

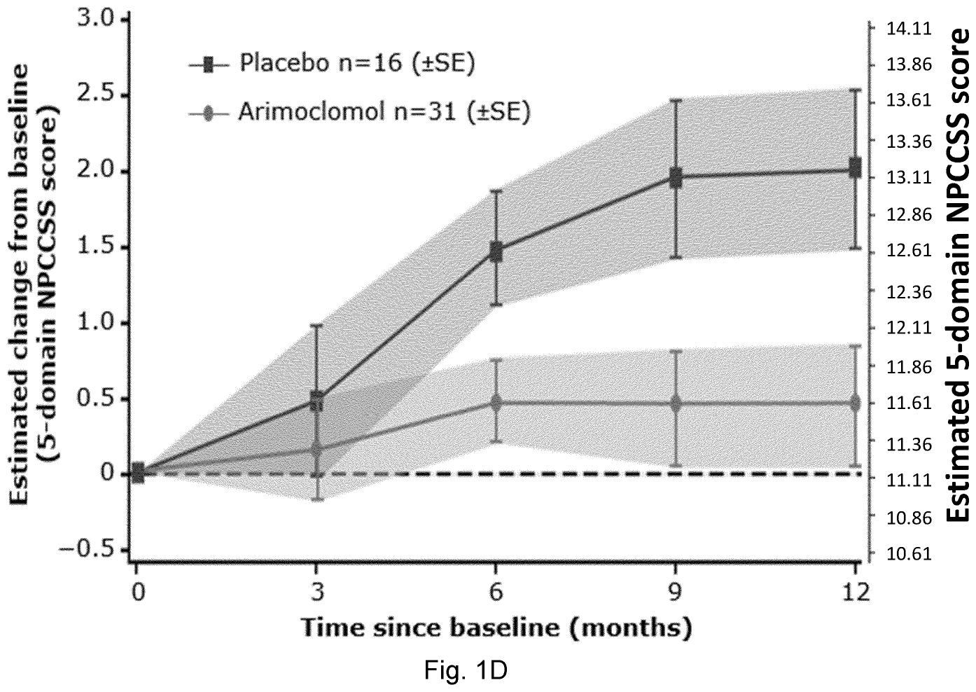

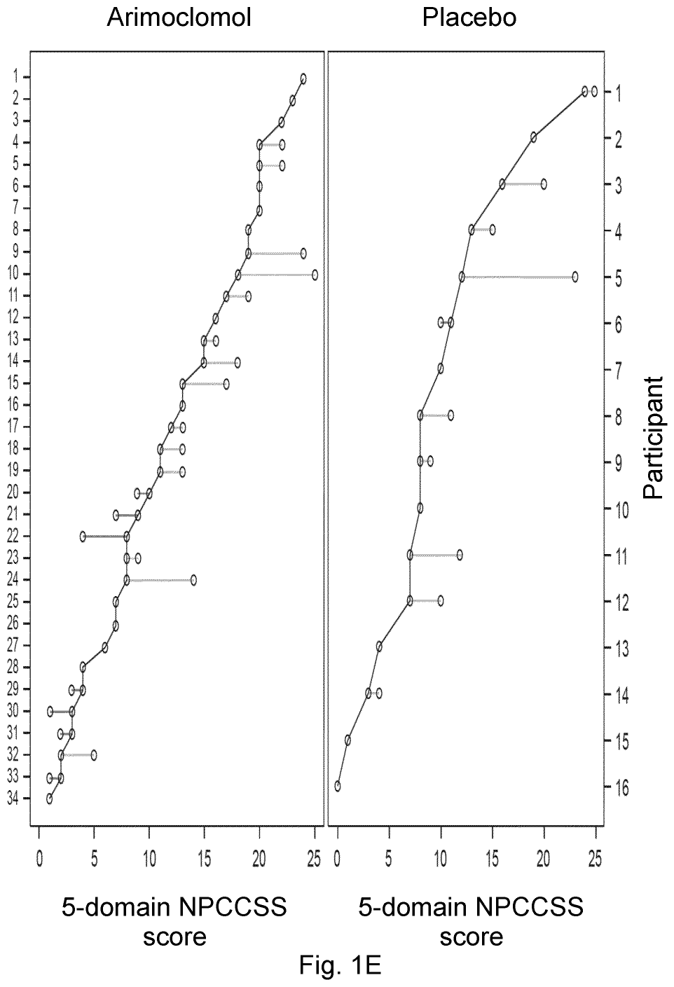

Efficacy Evaluation: For the primary endpoint, at month 12, mean (95% confidence interval [CI]) change on the 5-domain NPCCSS score was 0.80 (−0.01, 1.60) for arimoclomol compared with 2.14 (1.04, 3.24) for placebo, corresponding to a treatment effect in favor of arimoclomol of −1.34 (95% CI: −2.71, 0.02; p=0.0537; see FIG. 1A) and a 63% relative reduction in annual disease progression. Patient-level data for the change in 5-domain NPCCSS score are presented in FIG. 1E.

The primary multilevel modeling for repeated measures (MMRM) model was applied on the prespecified subgroup levels with enough patients to substantiate a formal analysis. In the subgroups of patients ≥4 years old (n=44; see FIG. 1B) and patients concomitantly receiving miglustat (n=39; FIG. 10), treatment with arimoclomol results in slowed progression of NPC (p<0.05; see Table 7).

TABLE 7
Change in clinical endpoints (full analysis set, except subgroup analyses).

| | | | | | Arimoclomol | |
|---|---|---|---|---|---|---|
| Change in 5-domain | | | | | vs placebo: | |
| NPCCSS score from | Arimoclomol | | Placebo | | Difference | |
| baseline at 12 months | (n = 34) | | (n = 16) | | (95% CI) | p value |
| Overall population, n (n at | 34 | (27) | 16 | (15) | | |
| 12 months) | | | | | | |
| Mean change (95% CI) | 0.80 | | 2.14 | | −1.34 | 0.0537 |
| | (−0.01, 1.60) | | (1.04, 3.24) | | (−2.71, 0.02) | |
| Relative reduction in annual | | | | | 63 | |
| disease progression, % | | | | | | |

Subgroup analyses

| Individuals receiving | 26 | (22) | 13 | (12) | | |
|---|---|---|---|---|---|---|
| miglustat, n (n at 12 | | | | | | |
| months) | | | | | | |
| Mean change (95% CI) | −0.01 | | 2.00 | | −2.01 | 0.0074 |
| | (−0.85, 0.83) | | (0.84, 3.15) | | (−3.44, −0.58) | |
| Relative reduction in annual | | | | | 101 | |
| disease progression, % | | | | | | |
| Individuals not receiving | 8 | (3) | 3 | (3) | | |
| miglustat, n (n at 12 | | | | | | |
| months) | | | | | | |
| Mean change (95% CI) | 4.2 | | 1.99 | | 2.21 | 0.2835 |
| | (1.7, 6.71) | | (−1.6, 5.57) | | (−2.14, 6.57) | |
| Relative reduction in annual | | | | | NA | |
| disease progression, % | | | | | | |
| Individuals aged ≥4 years, | 30 | (24) | 14 | (13) | | |
| n (n at 12 months) | | | | | | |
| Mean change (95% CI) | 0.44 | | 2.19 | | −1.75 | 0.0190 |
| | (−0.40, 1.28) | | (1.02, 3.37) | | (−3.20, −0.30) | |
| Relative reduction in annual | | | | | 80 | |
| disease progression, % | | | | | | |
| Individuals aged <4 years, | 3 | (3) | 2 | (2) | | |
| n (n at 12 months) | | | | | | |
| Mean change (95% CI) | NC | | NC | | | |
| Relative reduction in annual | | | | | NA | |
| disease progression, % | | | | | | |
| Responders on 5-domain | 17 | (50.0) | 6 | (37.5) | 12.5 | 0.5456 |
| NPCCSS score at 12 | | | | | (−16.6, 41.6) | |
| months, n (%) | | | | | | |
| Proportion worsening on 5- | 15 | (44.1) | 7 | (43.8) | 0.37 | 1.0000 |
| domain NPCCSS score at | | | | | (−29.1, 29.8) | |
| 12 months, n (%) | | | | | | |
| Time to worsening on 5- | 3.68 | | 4.29 | | NA | 0.8733 |
| domain NPCCSS score, | (2.89, 5.95) | | (1.94, 6.48) | | | |
| months (95% CI) | | | | | | |
| Full NPCCSS score | 25 | | 15 | | | |
| (excluding hearing | | | | | | |
| domains) at 12 months, n | | | | | | |
| Mean (SD) | 1.2 | (2.6) | 2.7 | (5.4) | | |
| Median (IQR) | 1.0 (−6.0 | | 0.0 | | | |
| | to 6.0) | | (−7.0, 13.0) | | | |
| LS mean change from | 1.24 (−0.35 | | 2.80 | | −1.56 | 0.5726 |
| baseline (95% CI) | to 2.84) | | (0.74, 4.86) | | (−4.18, 1.06) | |
| Responders on CGI-I at 12 | 20/34 | (58.8) | 9/16 | (56.3) | 2.6 | 1.0000 |
| months, n (%) | | | | | (−26.8, 32.0) | |
| NPC-cdb score change | 1.85 (−2.16 | | 4.88 | | −3.03 | 0.3785 |
| from baseline to 12 months, | to 5.86) | | (−0.63, 10.39) | | (−9.90, 3.85) | |
| LS mean (95% CI) | | | | | | |
| SARA score change from | 1.06 (−0.17 | | 0.78 | | 0.28 | 0.7899 |
| baseline to 12 months, LS | to 2.29) | | (−0.90, 2.47) | | (−1.82, 2.37) | |
| mean (95% CI) | | | | | | |

EQ-5D-3L Y proxy, n (%)

| | | | | | | |
|---|---|---|---|---|---|---|
| Improved at 12 months | 7/27 | (25.9) | 6/15 | (40.0%) | −14.1 | 0.4880 |
| | | | | | (−43.9, 15.7) | |
| Worsened at 12 months | 12/27 | (44.4) | 3/15 | (20.0%) | 24.4 | 0.1804 |
| | | | | | (−3.1, 52.0) | |

9-HPT time (s), change from baseline to 12 months, LS mean (95% CI)

| | | | | |
|---|---|---|---|---|
| Dominant hand | −3.29 | −6.49 | 3.20 | 0.7283 |
| | (−15.56, 8.98) | (−20.34, 7.37) | (−15.71, 22.12) | |
| Non-dominant hand | 11.68 | 17.59 | −5.91 | 0.7708 |
| | (−14.89, 38.25) | (−13.24, 48.42) | (−47.54, 35.72) | |

Based on 5-domain NPCCSS scores, the proportion of responders (stable or improved) was 50.0% and 37.5% in the arimoclomol and placebo groups, respectively (see Table 6). At 12 months, the mean difference in change from baseline in the 17-domain NPCCSS score (excluding hearing domain) and NPC-cdb scores for the arimoclomol versus placebo groups were numerically in favor of arimoclomol (p=0.5726 and p=0.3785, respectively; see Table 6). For the majority of patients (42/50), trial investigators completed baseline CGI-I assessments retrospectively.

To determine whether arimoclomol showed particular efficacy within certain subsets of the subjects in the study, additional post hoc analyses based on ASIS and genotype were performed. When analyzing patients that had a baseline ASIS of 0.5-2.0 (n=21), signal enhancement was observed with a treatment difference of −2.39 in favor of arimoclomol (see Table 7). In addition, when patients that had an NPC1 genotype of Functional Null/Functional Null were excluded from the analysis, there was a dramatically enhanced signal of treatment effect of −1.56 (see Table 8 and FIG. 1D).

TABLE 8
Change in clinical endpoints additional subgroup analyses.

| | | | Arimoclomol | |
|---|---|---|---|---|
| | | | vs placebo: | |
| | Arimoclomol | Placebo | Difference | p |
| | (n = 34) | (n = 16) | (95% CI) | value |
| Excluding individuals with | 31 | 16 | | |
| double null functional | | | | |
| mutations, n | | | | |
| Mean change (95% CI) | 0.47 | 2.03 | −1.56 | 0.0242 |
| | (−0.34, 1.28) | (0.96, 3.09) | (−2.90, −0.21) | |
| Relative reduction in | | | 77 | |
| annual disease | | | | |
| progression, % | | | | |
| ASIS within 0.5 and 2, n | 13 | 8 | | |
| Mean change (95% CI) | 0.19 | 2.58 | −2.39 | 0.0536 |
| | (−1.26, 1.63) | (0.65, 4.52) | (−4.83, 0.04) | |
| Relative reduction in | | | 93 | |
| annual disease | | | | |
| progression, % | | | | |

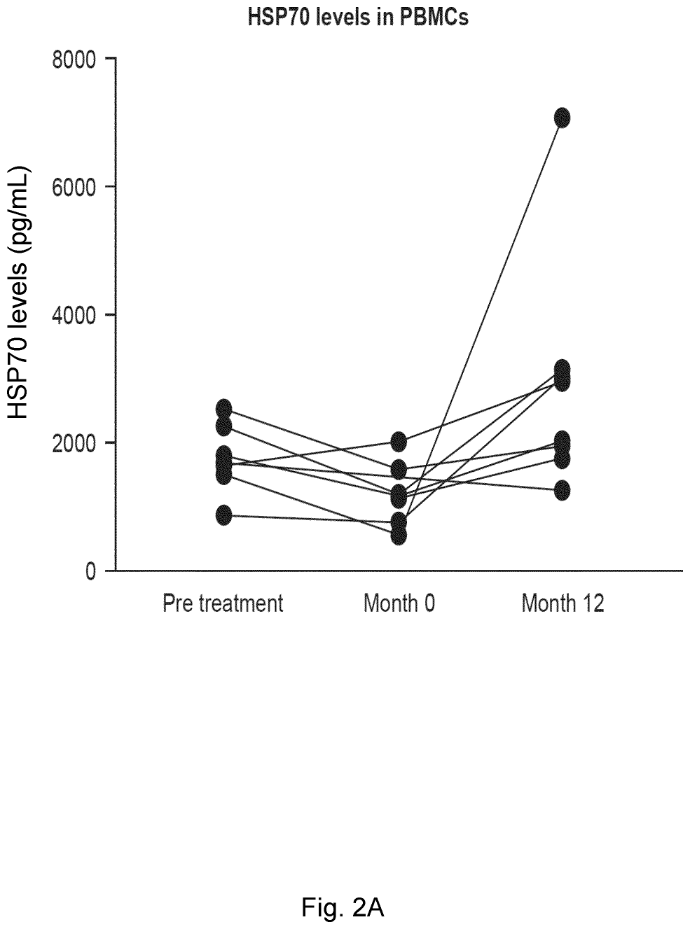

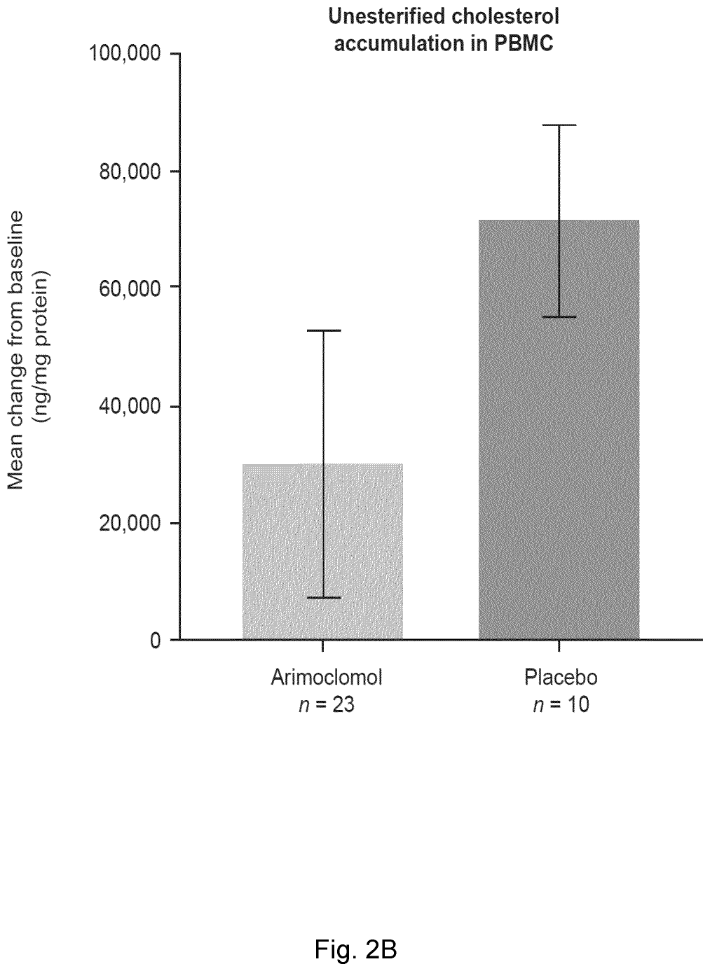

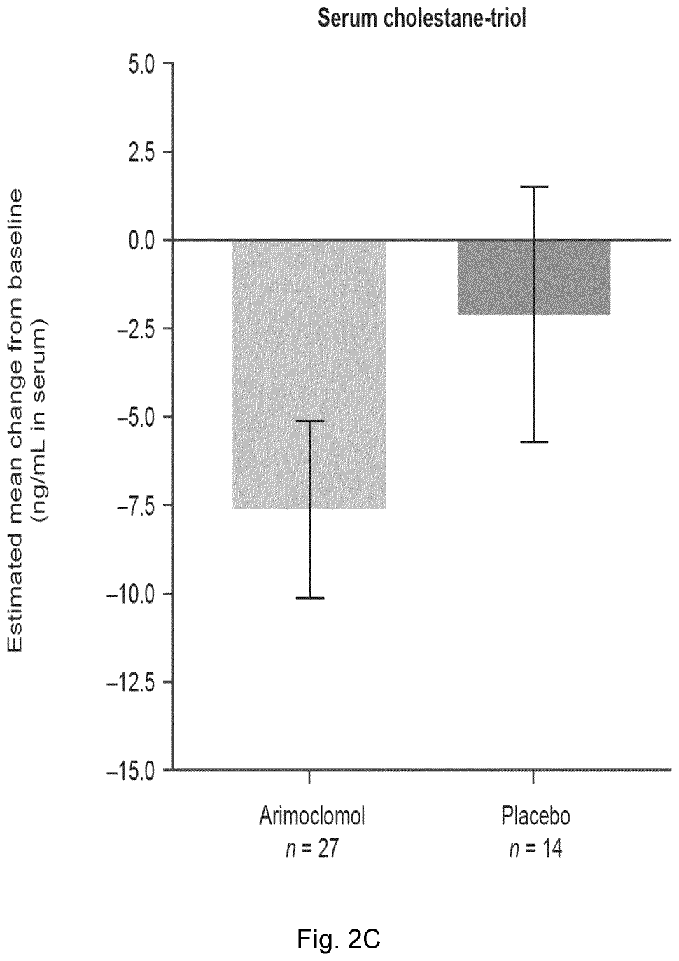

An increase in HSP70 level was observed in response to 12 months of treatment with arimoclomol (n=11; mean [standard deviation] change from baseline 1778.98 [1835.56] pg/mL; p=0.001; see FIG. 2A). Unesterified cholesterol levels in PBMCs increased from baseline to month 12 in both placebo- and arimoclomol-treated patients. The accumulation of unesterified cholesterol was numerically less in arimoclomol—than placebo-treated patients (mean treatment difference [standard error (SE)] −44.44 [25.83] μg/mg protein; p=0.096; see FIG. 2B). A numerical decrease in serum cholestane-triol level was observed in the arimoclomol group relative to the placebo group at 12 months (mean treatment difference [SE] −5.50 [4.46] ng/mL; p=0.225; see FIG. 2C).

Additionally, the treatment difference in favour of arimoclomol increased in the subpopulation of patients with an ER type missense mutation to −4.79 (95% CI, −7.83; −1.74), p=0.0053), representing a strong enhancement of signal in this genetically defined group (see Table 9).

TABLE 9
Analysis of change from baseline in 5-Domain NPCCSS
Score at 12 Months (Patients with ER Genotype)

| | | | |
|---|---|---|---|
| | | Treatment | |
| | LSMean | difference | |
| N | (95% CI) | (95% CI) | p-value |

Month 3

| | | | | |
|---|---|---|---|---|
| Arimoclomol | 11 | −0.29 | | |
| | | (−1.48; 0.89) | | |
| Placebo | 4 | 1.31 | −1.60 | 0.1558 |
| | | (−0.67; 3.28) | (−3.91; 0.71) | |

Month 6

| | | | | |
|---|---|---|---|---|
| Arimoclomol | 10 | 0.61 | | |
| | | (0.21; 1.01) | | |
| Placebo | 4 | 2.81 | −2.19 | <.0001 |
| | | (2.16; 3.45) | (−2.98; −1.41) | |

Month 9

| | | | | |
|---|---|---|---|---|
| Arimoclomol | 9 | 0.86 | | |
| | | (−0.88; 2.61) | | |
| Placebo | 4 | 4.31 | −3.44 | 0.0350 |
| | | (1.68; 6.93) | (−6.59; −0.29) | |

Month 12

| | | | | |
|---|---|---|---|---|
| Arimoclomol | 9 | 0.52 | | |
| | | (−1.14; 2.18) | | |
| Placebo | 4 | 5.31 | −4.79 | 0.0053 |
| | | (2.76; 7.85) | (−7.83; −1.74) | |

N: Number of patients contributing to the analysis,
CI: Confidence interval
The estimates are from a mixed model for repeated measures modelling month 3, 6, 9 and 12 with treatment, use of miglustat, visit and treatment-by-visit interaction as fixed effects and baseline value as covariate.

Safety Evaluation: In total, 88.2% (30/34) of patients in the arimoclomol group and 75.0% (12/16) in the placebo group had TEAEs. The most common TEAE in both treatment groups was vomiting (arimoclomol: 8/34, 23.5%; placebo: 4/16, 25.0%). Upper respiratory tract infection and decreased weight occurred more frequently with arimoclomol versus placebo, whereas nasopharyngitis, respiratory tract infection, and epilepsy were reported more often with placebo versus arimoclomol. Serious TEAEs occurred in 14.7% (5/34) of patients receiving arimoclomol compared with 31.3% (5/16) of those receiving placebo. All serious TEAEs except those leading to discontinuation from the trial were considered related to NPC. One patient died owing to cardiopulmonary arrest assessed as being related to NPC and not to treatment with arimoclomol. Three patients in the arimoclomol group (8.8%) had four TEAEs that led to trial drug discontinuation. These included two events of urticaria and one of angioedema (all classed as serious and as probably related to investigational product), and one of increased blood creatinine level twice the patient's baseline value (assessed as being related to investigational product). Six patients in the arimoclomol group had an increase in serum creatinine level over 1.5 times their baseline values; for two of these patients (both in the arimoclomol group), levels were over twice baseline values. For one of these patients, the increase occurred during treatment with rescue medication; for the other the elevation was reported as a TEAE and the patients discontinued involvement in the trial, in line with the protocol. None of the patients had any other indications of affected kidney function. For all patients, the creatinine level started to rise at the first measurement after exposure to arimoclomol and was seen to peak before the end of the trial. There were no significant changes in vital signs, electrocardiograms, or other laboratory values during the trial.

### Conclusion

Without wishing to be bound by theory, the results of the placebo-controlled phase 2/3, 12-month clinical trial of arimoclomol in NPC described above demonstrates that arimoclomol was well tolerated with clinically meaningful benefit of arimoclomol versus placebo observed in the tested subjects. Significant reduction of disease progression was observed in patients over four years of age and in those concomitantly treated with miglustat. Moreover, arimoclomol treatment demonstrated increased efficacy in subjects that did not have an NPC1 genotype of Functional Null/Functional Null (i.e. subjects with an NPC1 genotype of either Missense/Missense or Missense/Functional Null) and in patients from the ER type subgroup.

### Example 2—In vitro Studies of Cell Lines Treated With Arimoclomol

This experiment investigated the effects of EndoH (Endoglycosidase H) deglycosylation treatment on the NPC1 protein band in Western blot analysis of PBS- and arimoclomol-treated human fibroblast cell lines with missense mutations affecting ER trafficking (GM18453; I1061T/I1061T) and lysosomal localization (GM18420; P1007A/IVS23+4delA) and was performed three times.

### Materials and Methods

### Cell Treatment

Fibroblasts were seeded one day prior to treatment (day −1) with either 400 μM arimoclomol or PBS (control). Cells were treated continuously with arimoclomol from day 0 to day 5, with a media change on day 3, and harvested on day 5. The cells thus experienced 5 days of continued arimoclomol treatment. Cells were subsequently lysed with RIPA buffer containing protease and phosphatase inhibitors. Protein concentration for each sample was quantified with a BCA assay kit.

### Enzymatic Digestion

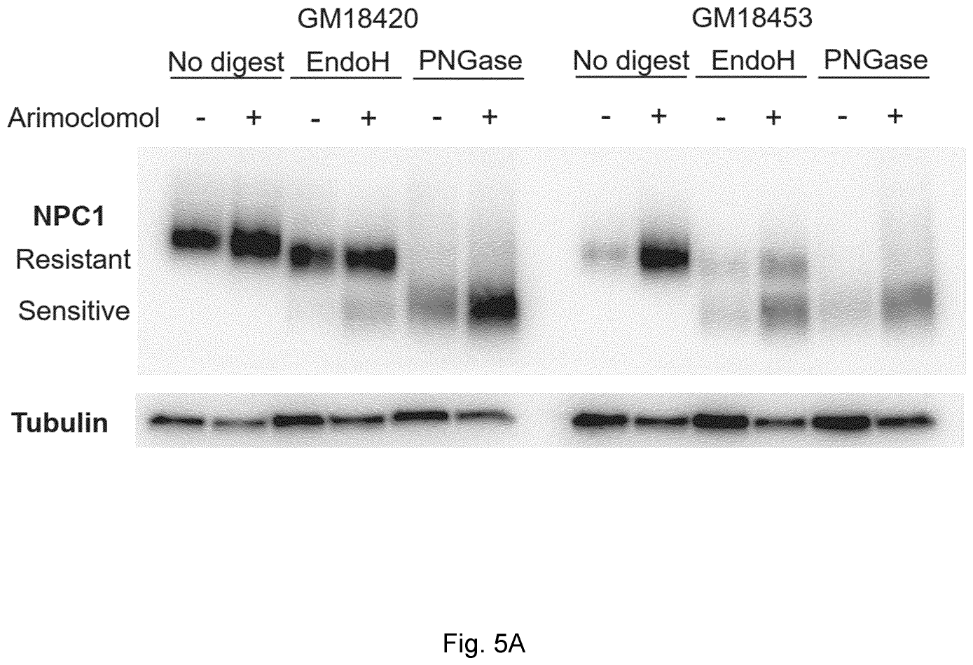

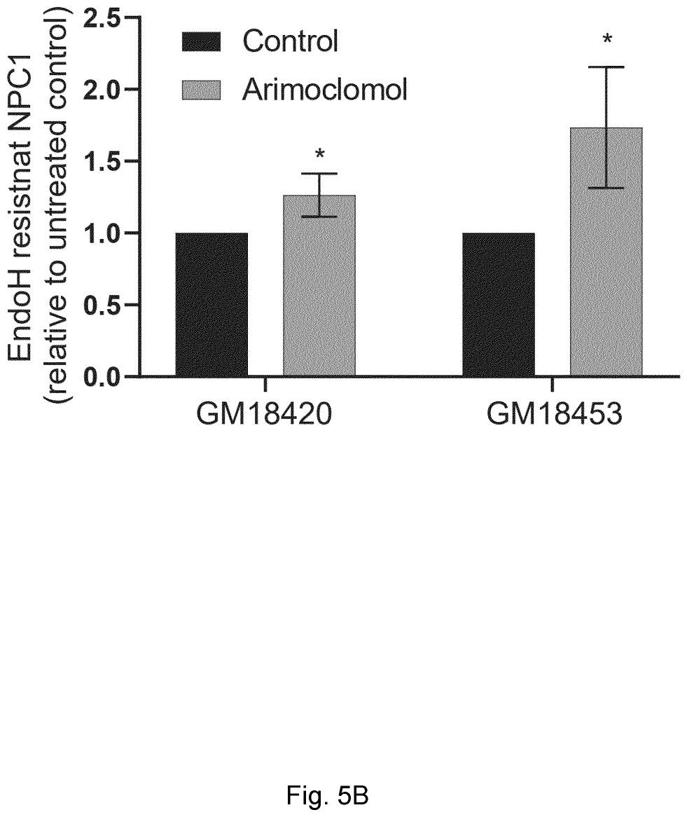

Proteins (10 μg protein/sample) were treated with the appropriate enzyme (see Table 10 below and FIG. 5A). Unlike EndoH, which specifically removes glycans from immature proteins in the ER, peptide N glycosidase F (PNGase) removes all glycans and therefore served as a positive digestion control.

TABLE 10
Enzymatic digestion sample layout

| Cell line | Treatment | Digestion treatment |
|---|---|---|
| GM18420 | PBS (control) | No digestion (PBS) |
| GM18420 | Arimoclomol (400 μM) | No digestion (PBS) |
| GM18420 | PBS (control) | EndoH |
| GM18420 | Arimoclomol (400 μM) | EndoH |
| GM18420 | PBS (control) | PNGase |
| GM18420 | Arimoclomol (400 μM) | PNGase |
| GM18453 | PBS (control) | No digestion (PBS) |
| GM18453 | Arimoclomol (400 μM) | No digestion (PBS) |
| GM18453 | PBS (control) | EndoH |
| GM18453 | Arimoclomol (400 μM) | EndoH |
| GM18453 | PBS (control) | PNGase |
| GM18453 | Arimoclomol (400 μM) | PNGase |

### SDS-PAGE and Western Blot

Proteins were resolved by SDS-PAGE and detected with Western blot (NPC1&tubulin).

### Image Quantification and Statistical Analysis

For the quantification of mature and immature forms of NPC1 as shown in FIGS. 5A-B, membranes were imaged and protein bands for the immature (EndoH sensitive) and mature (EndoH resistant) forms of NPC1 (180-250 kDa) and tubulin (50 kDa) were quantified using ImageJ software. NPC1 bands (EndoH-resistant and EndoH-sensitive) were normalized to tubulin. The fold change of EndoH-resistant NPC1 protein after arimoclomol treatment relative to PBS-treated (untreated) control within each cell line was quantified. Data are presented as means+/−standard deviation. Statistical significance was conducted using paired t tests (control versus arimoclomol) for each cell line.

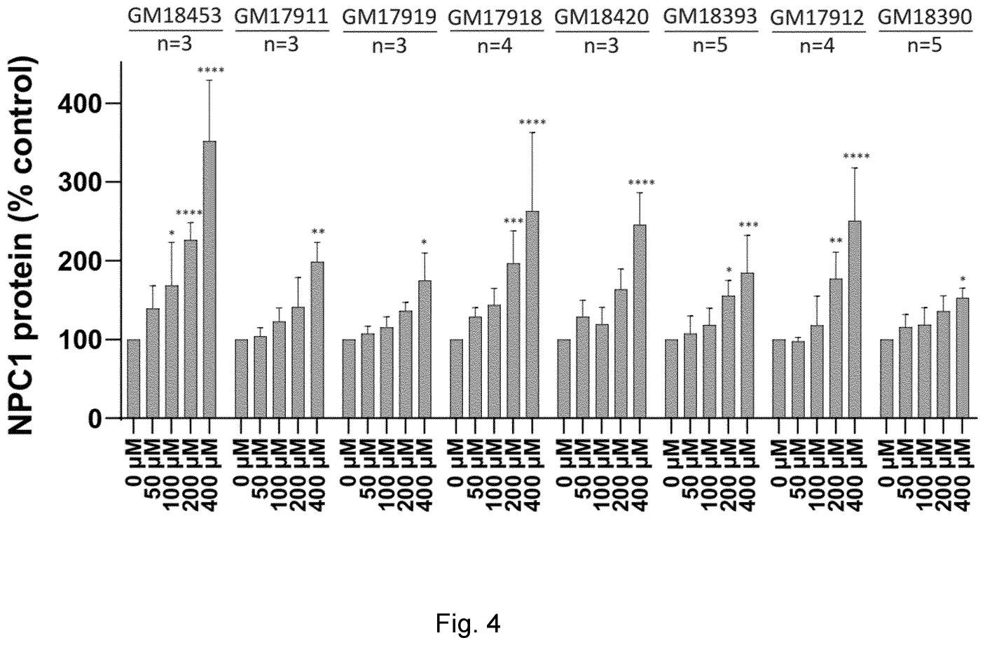

For the determination of NPC1 protein abundance as shown in FIG. 4, membranes were imaged and protein bands for NPC1 (180-250 kDa) and tubulin (50 kDa) were quantified using ImageJ software. NPC1 bands were normalized to tubulin or ponceau staining. The fold change total NPC1 protein after arimoclomol treatment relative to PBS-treated (untreated) control within each cell line was quantified. Data are presented as means+/−standard deviation. Statistical significance was conducted using paired t tests (control versus arimoclomol) for each cell line. The NPC1 genotype of each cell line is shown in Table 11.

TABLE 11
NPC1 genotypes of fibroblasts cell lines in FIG. 4.

| Cell line name | Allele 1 | Allele 2 |
|---|---|---|
| GM18453 | I1061T | I1061T |
| GM17911 | I1061T | T1036M |
| GM17919 | I1061T | R404W |
| GM17918 | T137M | c.2336insT |
| GM18393 | G248V | M1142T |
| GM18390 | D242H | S940L |
| GM18420 | P1007A | g.IVS23 + 4delA |
| GM17912 | P1007A | T1036M |

### Results and Conclusion

Transient expression of NPC1 cDNA-encoding tagged NPC1 mutant protein in mammalian cell lines is frequently used as a system to model the transport out of the ER, in combination with the endoglycosidase H (EndoH) enzyme which digests immature glycosylation structures specifically on proteins retained in the ER. In these experimental conditions almost all I1061T NPC1 protein is sensitive to EndoH, indicating a strong blockade of exit from the ER, and several other mutations found in this study have been shown to have a similar phenotype (Shammas et al. 2019).

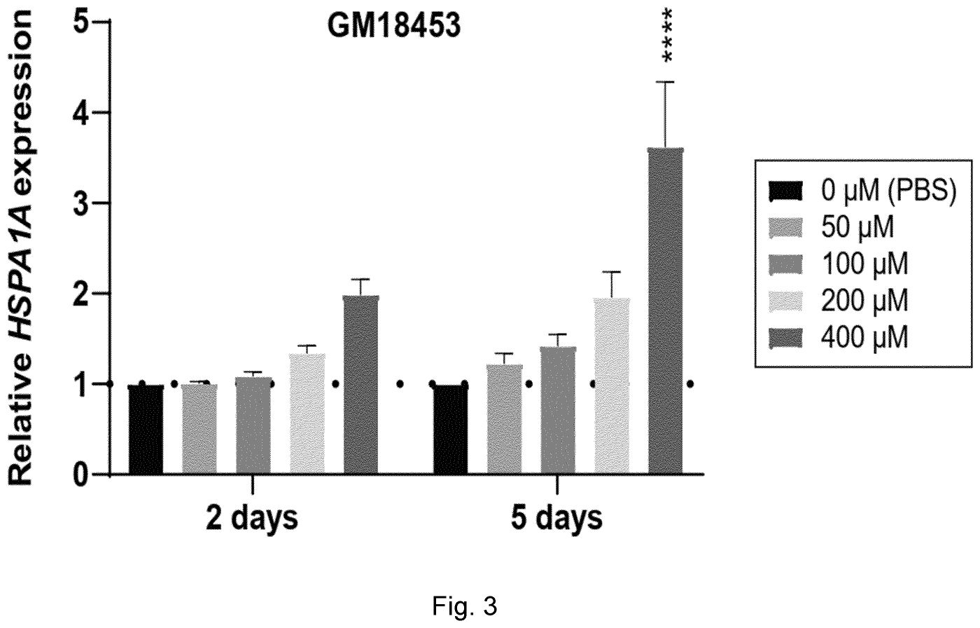

Treatment of NPC patient fibroblasts with ER type mutations (I1061T/I1061T) with arimoclomol resulted in a time- and dose-dependent increase in expression of HSPA1A, the gene encoding HSP70, after 5 days relative to phosphate buffered saline (PBS)-treated control (see FIG. 3).

Treatment with arimoclomol increased the amount of total NPC1 protein relative to PBS-treated control in patient fibroblasts with the most common mutations in NPC1 (see FIG. 4).

Arimoclomol increased the amount of correctly processed NPC1 protein as quantified by the amount of EndoH-resistant NPC1 relative to PBS-treated cells in NPC fibroblasts with missense mutants affecting ER trafficking (I1061T; GM18453) and lysosomal localization (P1007A; GM18420) (see FIG. 5A).

EndoH specifically removes glycans from immature proteins in the ER, allowing differentiation between immature (sensitive) and mature (resistant) forms of NPC1. Treatment with arimoclomol increased the amount of properly processed NPC1 protein as quantified by the amount of EndoH-resistant NPC1 relative to PBS-treated cells in NPC fibroblasts with missense mutations affecting ER trafficking (I1061T; GM18453) and lysosomal localization (P1007A; GM18420) (see FIG. 5B).

The mutations of GM18453 result in an NPC1 protein that is misfolded, retained at the endoplasmic reticulum (ER) and subsequently targeted for degradation. Importantly, HSP70 has been shown to directly bind to I1061T NPC1 and aid in the proper folding and maturation of the protein. As evident from these data, arimoclomol treatment of patient fibroblasts with an ER type missense mutation leads to a clear increase of correctly matured NPC1 protein.

Thus, patients with at least one ER type mutation would be expected to benefit both from arimoclomol's beneficial effect on lysosomal homeostasis and heat shock protein (HSP) dependent refolding, and maturation of misfolded NPC1 retained in the ER.

### REFERENCES

Busner and Targum. “The clinical global impressions scale: applying a research tool in clinical practice”. Psychiatry (Edgmont). 2007; 4:28-37
Cortina-Borja et al. “Annual severity increment score as a tool for stratifying patients with Niemann-Pick disease type C and for recruitment to clinical trials.” Orphanet J Rare Dis, 2018; 13 (1): 143; 3
Imrie et al. 2007. “The Natural History of Niemann-Pick Disease Type C in the UK.” Journal of Inherited Metabolic Disease 30 (1): 51-59.
   https://doi.org/10.1007/s10545-006-0384-7.
Gelsthorpe et al. 2008. “Niemann-Pick Type C1 I1061T Mutant Encodes a Functional Protein That Is Selected for Endoplasmic Reticulum-Associated Degradation Due to Protein Misfolding.” The Journal of Biological Chemistry 283 (13): 8229-36.
Mengel et al. “Clinical disease progression and biomarkers in Niemann-Pick disease type C: a prospective cohort study.” Orphanet J Rare Dis. 2020; 15:328
Poole et al. “Measuring dexterity in children using the Nine-hole Peg Test”. J Hand Ther. 2005; 18:348-51
Schmitz-Hubsch et al. “Scale for the assessment and rating of ataxia: development of a new clinical scale”. Neurology. 2006; 66:1717-20
Schultz et al. 2018. “Coordinate Regulation of Mutant NPC1 Degradation by Selective ER Autophagy and MARCH6-Dependent ERAD.” Nature Communications 9 (1): 3671
Shammas et al. 2019. “Different Niemann-Pick C1 Genotypes Generate Protein Phenotypes That Vary in Their Intracellular Processing, Trafficking and Localization.” Scientific Reports 9 (1): 5292.
Stampfer et al. “Niemann-Pick disease type C clinical database: cognitive and coordination deficits are early disease indicators.” Orphanet J Rare Dis. 2013; 8:35 doi: 10.1186/750-72-8-35
Wang et al. “Individualized management of genetic diversity in Niemann-Pick C1 through modulation of the Hsp70 chaperone system.” Human molecular genetics vol. 29, 1 (2020): 1-19. doi:10.1093/hmg/ddz215
Yanjanin et al. “Linear Clinical Progression, Independent of Age of Onset, in Niemann-Pick Disease, type C.” Am J Med Genet B Neuropsychiatr Genet, 2010; 153B (1): 132-140

1-64. (canceled)

65. A method of treating Niemann Pick disease, type C (NPC) in a subject in need thereof, the method comprising administering to the subject an active pharmaceutical ingredient selected from the group consisting of N-[2-hydroxy-3-(1-piperidinyl)-propoxy]-pyridine-1-oxide-3-carboximidoyl chloride, stereoisomers of N-[2-hydroxy-3-(1-piperidinyl)-propoxy]-pyridine-1-oxide-3-carboximidoyl chloride, acid addition salts of N-[2-hydroxy-3-(1-piperidinyl)-propoxy]-pyridine-1-oxide-3-carboximidoyl chloride and acid addition salts of the stereoisomers, and wherein the subject has an endoplasmic reticulum (ER) type missense mutation in at least one NPC gene, wherein the at least one NPC gene is selected from the group consisting of NPC1, NPC2, and combinations thereof.

66. The method of claim 65, wherein said active pharmaceutical ingredient is (+)-(R)-N-[2-hydroxy-3-(1-piperidinyl)-propoxy]-pyridine-1-oxide-3-carboximidoyl chloride or the acid addition salts thereof.

67. The method of claim 66, wherein said active pharmaceutical ingredient is (+)-(R)-N-[2-hydroxy-3-(1-piperidinyl)-propoxy]-pyridine-1-oxide-3-carboximidoyl chloride citrate.

68. The method of claim 65, further comprising administering a further active pharmaceutical ingredient selected from the group consisting of an N-alkyl derivative of 1,5-dideoxy-1,5-imino-D-glucitol in which said alkyl contains from 2-8 carbon atoms, stereoisomers of an N-alkyl derivative of 1,5-dideoxy-1,5-imino-D-glucitol in which said alkyl contains from 2-8 carbon atoms, and acid addition salts thereof.

69. The method of claim 68 wherein said further active pharmaceutical ingredient is N-butyl-deoxynojirimycin (miglustat).

70. A method of treating Niemann Pick disease, type C (NPC) in a subject in need thereof, comprising administering to the subject (+)-(R)-N-[2-hydroxy-3-(1-piperidinyl)-propoxy]-pyridine-1-oxide-3-carboximidoyl chloride citrate (arimoclomol), in combination with N-butyl-deoxynojirimycin (miglustat), wherein the subject has at least one ER type missense mutation in at least one NPC gene, wherein the at least one NPC gene is selected from the group consisting of NPC1, NPC2, and combinations thereof.

71. The method of claim 65 or 70, wherein the at least one NPC gene is NPC1.

72. The method of claim 65 or 70, wherein the at least one ER type missense mutation results in a single amino-acid change.

73. The method of claim 72, wherein the at least one ER type missense mutation is selected from the group consisting of C113R, R389L, G535V, L724P, Q921P, W942C, G1034C, V378A, R404Q, H510P, Q775P, M1142T, N1156S, G1162V, R1186H, L1244P, I1061T and combinations thereof.

74. The method of claim 73, wherein the at least one ER type missense mutation is selected from the group consisting of I1061T, M1142T, N1156S, R1186H and combinations thereof.

75. The method of claim 74, wherein the at least one ER type missense mutation is I1061T.

76. The method of claim 70 or 73, wherein the subject has an NPC1 genotype selected from the group consisting of I1061T/E1188*, I1061T/A1151T, I1061T/Q119fs, I1061T/I962fs, T1036M/I1061T, I1061T/V1141G, N968S/R1186H, N1156S/F1199sp2, Q991fs/I1061T, H1016L/I1061T, I1061T/A1192fs, I1061T/N1156S, R1186H/R1186H, P1007A/R1186H, and I1061T/D508fs.

77. The method of claim 67 or 70, wherein the (+)-(R)-N-[2-hydroxy-3-(1-piperidinyl)-propoxy]-pyridine-1-oxide-3-carboximidoyl chloride citrate is administered from about 100 mg/day to about 1000 mg/day.

78. The method of claim 77, wherein (+)-(R)-N-[2-hydroxy-3-(1-piperidinyl)-propoxy]-pyridine-1-oxide-3-carboximidoyl chloride citrate is administered at about 100 mg/day to about 700 mg/day.

79. The method of claim 78, wherein the (+)-(R)-N-[2-hydroxy-3-(1-piperidinyl)-propoxy]-pyridine-1-oxide-3-carboximidoyl chloride citrate active pharmaceutical ingredient is administered at about 150 mg/day to about 600 mg/day.

80. The method of claim 67 or 70, wherein the (+)-(R)-N-[2-hydroxy-3-(1-piperidinyl)-propoxy]-pyridine-1-oxide-3-carboximidoyl chloride citrate is administered in doses of about 25 mg to about 300 mg.

81. The method of claim 80, wherein the (+)-(R)-N-[2-hydroxy-3-(1-piperidinyl)-propoxy]-pyridine-1-oxide-3-carboximidoyl chloride citrate is administered in doses of about 50 mg, to about 200 mg.

82. The method of claim 81, wherein the (+)-(R)-N-[2-hydroxy-3-(1-piperidinyl)-propoxy]-pyridine-1-oxide-3-carboximidoyl chloride citrate is administered at least one day a week, at least two days a week, at least three days a week, at least four days a week, at least five days a week, at least six days a week, or at least seven days a week.

83. The method of claim 82, wherein the (+)-(R)-N-[2-hydroxy-3-(1-piperidinyl)-propoxy]-pyridine-1-oxide-3-carboximidoyl chloride citrate is administered at least one time daily to at least five times daily.

84. The method of claim 82 wherein the (+)-(R)-N-[2-hydroxy-3-(1-piperidinyl)-propoxy]-pyridine-1-oxide-3-carboximidoyl chloride citrate is administered two times daily (b.i.d.) or three times daily (t.i.d.).

85. The method of claim 80, wherein the (+)-(R)-N-[2-hydroxy-3-(1-piperidinyl)-propoxy]-pyridine-1-oxide-3-carboximidoyl chloride citrate is administered orally.

86. The method of claim 85, wherein the (+)-(R)-N-[2-hydroxy-3-(1-piperidinyl)-propoxy]-pyridine-1-oxide-3-carboximidoyl chloride citrate is formulated for oral administration as a tablet, capsule, oral powder, an oral powder suitable for suspension in a liquid or a suspension.

87. The method of claim 67 or 70 wherein the subject is a mammal.

88. The method of claim 87, wherein the mammal is a human.

89. A method of treating Niemann Pick disease, type C (NPC) in a subject in need thereof, the method comprising administering to the subject a therapeutically effective amount of an active pharmaceutical ingredient selected from the group consisting of N-[2-hydroxy-3-(1-piperidinyl)-propoxy]-pyridine-1-oxide-3-carboximidoyl chloride, stereoisomers of N-[2-hydroxy-3-(1-piperidinyl)-propoxy]-pyridine-1-oxide-3-carboximidoyl chloride, acid addition salts of N-[2-hydroxy-3-(1-piperidinyl)-propoxy]-pyridine- 1-oxide-3-carboximidoyl chloride and acid addition salts of the stereoisomers, wherein the subject has at least one ER type missense mutation in at least one NPC gene, and wherein the subject is identified as having the at least one ER type missense mutation in at least one of the two alleles of the at least one NPC gene.

90. The method of claim 89, wherein the subject is identified as having at least one ER type missense mutation in each of the two alleles of the at least one NPC gene.

91. The method of claim 89 or 90, wherein the active pharmaceutical ingredient is (±)-(R)-N-[2-hydroxy-3-(1-piperidinyl)-propoxy]-pyridine-1-oxide-3-carboximidoyl chloride citrate.

92. The method of claim 89 or 90, further comprising administering a therapeutically effective amount of miglustat to the subject.

93. The method of claim 92, wherein the therapeutically effective amount of miglustat is administered from about 300 mg/day to about 1000 mg/day.

94. The method of claim 93, wherein the therapeutically effective amount of miglustat is administered from about 300 mg/day to about 600 mg/day.

95. A method of treating Niemann Pick disease, type C (NPC) in a subject in need thereof, the method comprising administering to the subject a therapeutically effective amount of (+)-(R)-N-[2-hydroxy-3-(1-piperidinyl)-propoxy]-pyridine-1-oxide-3-carboximidoyl chloride citrate (arimoclomol), in combination with a therapeutically effective amount of N-butyl-deoxynojirimycin (miglustat), wherein the subject has at least one ER type missense mutation in at least one NPC gene, and wherein the subject is identified as having the at least one ER type missense mutation in at least one of the two alleles of the at least one NPC gene.


SEQUENCE LISTING (2 sequences)

SEQ ID NO: 1 - DNA, 4760 bp, Homo Sapiens
cttcctgaccggcgcgcgcagcctgctgccgcggtcagcgcctgctcctgctcctccgctcctcctgcgcggggtgctgaaacagcccggggaagtagagccgcctccggggagcccaaccagccgaacgccgccggcgtcagcagccttgcgcggccacagcatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatgaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctagccctctcgcagggcatcctgactgaactgtgtctaagggtcggtcggtttaccactggacgggtgctgcatcggcaaggccaagttgaacaccggatggtgccaaccatcggttgtttggcagcagctttgaacgtagcgcctgtgaactcaggaatgcacagttgacttgggaagcagtattactagatctggaggcaaccacaggacactaaacttctcccagcctcttcaggaaagaaacctcattctttggcaagcaggaggtgacactagatggctgtgaatgtgatccgctcactgacactctgtaaaggccaatcaatgcactgtctgtctctccttttaggagtaagccatcccacaagttctataccatatttttagtgacagttgaggttgtagatacactttataacattttatagtttaaagagctttattaatgcaataaattaactttgtacacatttttatataaaaaaacagcaagtgatttcagaatgttgtaggcctcattagagcttggtctccaaaaatctgtttgaaaaaagcaacatgttcttcacagtgttcccctagaaaggaagagatttaattgccagttagatgtggcatgaaatgagggacaaagaaagcatctcgtaggtgtgtctactgggttttaacttatttttctttaataaaatacattgttttcctaagttttggggttaccctatctgctttgagagacaaatacaaaagctaaatggaagaga

SEQ ID NO: 2 - protein, 1278 aa, Homo Sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF